(19) United States
(12) Patent Application Publication
(10) Pub. No.: US 2024/0199608 A1
(43) Pub. Date: Jun. 20, 2024

(54) AHR AGONISTS

(71) Applicant: Eli Lilly and Company, Indianapolis, IN (US)

(72) Inventors: Michael Gregory BELL, Fishers, IN (US); Joel Robert CALVIN, Westfield, IN (US); Cheryl Ann CARSON, Indianapolis, IN (US); Lacie Marie CHAUVIGNE-HINES, Plainfield, IN (US); Christian Alexander CLARKE, Fishers, IN (US); Frank Charles DORSEY, Carmel, IN (US); Douglas Linn GERNERT, Indianapolis, IN (US); Steven James GREEN, Indianapolis, IN (US); William Thomas MCMILLEN, Indianapolis, IN (US); Antonio NAVARRO, Indianapolis, IN (US); John Robert RIZZO, Plainfield, IN (US)

(21) Appl. No.: 18/501,175

(22) Filed: Nov. 3, 2023

Related U.S. Application Data
(60) Provisional application No. 63/425,442, filed on Nov. 15, 2022.

(51) Int. Cl. C07D 471/04

(52) U.S. Cl. CPC C07D 471/04

(57) ABSTRACT

The present disclosure relates to certain AHR agonist compounds, their method of making, and to pharmaceutical compositions comprising the compounds and to methods of using the compounds to treat immune-mediated diseases, such as psoriasis and atopic dermatitis.

The present disclosure relates to novel AHR agonist compounds, to pharmaceutical compositions comprising the compounds, and to methods of using the compounds to treat certain immune-mediated diseases.

The present disclosure is in the field of treatment of certain immune-mediated diseases (IMD), for example psoriasis and atopic dermatitis, via the activation of the aryl hydrocarbon receptor (AHR).

IMDs encompass a broad range of chronic and debilitating inflammatory conditions that affect approximately 4% of the population worldwide. In view of the limited efficacy of currently available treatments, and few small molecule treatment options, there is significant unmet need for alternative, potent, selective, and safer drugs for the treatment of IMDs.

AHR is a transcription factor which regulates many aspects of immunological function, most notably the suppression of adaptive immune responses (Ehrlich et al., Curr. Opin. Toxicol., 2, 72-78 (2017)). Prototypical AHR agonists include halogenated dibenzodioxins, such as 2,3,7,8-tetrachlorodibenzodioxin (TCDD), tryptophan metabolites, such as L-kynurenine, bilirubin, and PGE2. Results from studies on AHR agonists, especially TCDD, suggest that immune suppression occurs as the result of AHR-induced expression of regulatory T cells (Tregs), TH17 cells, and dendritic cells (DCs) (Rothhammer et al., Nat. Rev, Immunol., 19, 184-197 (2019)). TCDD has been shown to be effective in the prevention of several murine models of IMD, including type-1 diabetes (Kerkvliet et al., Immunotherapy, 1, 539-547 (2009)), autoimmune encephalomyelitis (Quintana et al., Nature, 453, 65-71, (2008)), autoimmune uveoretinitis (Zhang et al., Invest. Opthalmol. Vis. Sci., 51, 2109-2117 (2010)), inflammatory bowel disease (Takamura et al., Immunol. Cell. Biol., 88, 685-689 (2010), Benson et al., Toxicol. Sci., 120, 68-78 (2011), Singh et al., PLoS One, 6(8), e23522 (2011)), as well as several models of transplant tolerance (Pauly at al., Toxicol. Environ. Chem., 94, 1175-1187 (2012)) and allergic diseases (Schulz et al., Toxicol. Sci., 123, 491-500 (2011), Li et al, PLoS One, 11, e0150551 (2016), Luebke et al., Toxicol. Sci., 62, 71-79 (2001)).

AHR also regulates the expression of CYP1A1, CYP1A2, and CYP1B1, which catalyze the metabolism of polycyclic aromatic hydrocarbon (PAH) and other aromatic compounds (e.g., estrogen). While in some cases (for example in the case of benzo[a]pyrene) this metabolism results in the formation of reactive species, CYP induction is also believed to be critical for the detoxification and metabolic clearance of PAHs, which reduces the probability of bioactivation, and DNA adduct formation. Several marketed drugs were found to activate AHR (thus upregulating CYP1A1, CYP1A2, and CYP1B1) after their FDA approval, yet their long-term use is not associated with dioxin-like toxicities (Ehrlich et al., Curr. Opin. Toxicol., 2, 72-78 (2017)). As such, CYP induction is no longer viewed as a barrier to the adoption of AHR agonists in therapy (Ehrlich et al., Curr. Opin. Toxicol., 2, 72-78 (2017)).

WO 2008/014307 discloses certain bicyclic heteroaryl amides as inhibitors of undecaprenyl pyrophosphate synthase. EP 0059698 discloses certain heterocyclic carboxamides, compositions containing these compounds and methods of treatment with these compositions.

The bacterial stilbenoid DMVT-505 (VTAMA® (tapinarof)) 1% cream, an aryl hydrocarbon receptor agonist, indicated for the topical treatment of plaque psoriasis in adults, has been approved by the U.S. Food and Drug Administration (FDA). Despite this, there remains a need for alternative novel, oral, selective, and/or potent AHR agonists for the treatment of IMDs. The present disclosure provides certain compounds that are agonists of AHR.

Accordingly, the present disclosure provides, for example, various embodiments as presented below. When a later embodiment refers to a previous “embodiment X”, such reference also includes references to “embodiment XA”, “embodiment XB”, and so on, unless such later embodiment cannot be properly construed as a dependent embodiment (e.g. falling outside the scope of the referenced embodiment or having improper antecedent basis). For example, when “Embodiment 54” below refers to “embodiment 53”, such reference also includes reference to “embodiment 53A”, “embodiment 53B”, “embodiment 53C”, “embodiment 53D”, and “embodiment 53E” among others.

Embodiment 1A. A compound of formula:

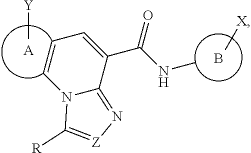

wherein:
   ring A is a 5-membered or 6-membered carbocycle;
   ring B is a phenyl, or a 5-membered or 6-membered heteroaryl having 1 or 3 heteroatoms, wherein each heteroatom of the heteroaryl is independently selected from N, S, and O;
   R is H or C₁₋₃ alkyl;
   X is H, halo, C₁₋₃ alkyl optionally substituted with one or more halo, or a C₁₋₃ alkoxyl;
   Y is H, C₁₋₄ alkyl, or C₃₋₄ cycloalkyl, wherein the C₁₋₄ alkyl is a primary or secondary alkyl; and
   Z is CH or N,
a stereoisomer or mixture of stereoisomers thereof, or a pharmaceutically acceptable salt of each thereof.

Embodiment 1. A compound of formula:

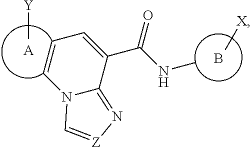

wherein:
ring A is a 5-membered or 6-membered carbocycle;
ring B is a phenyl, or a 5-membered or 6-membered heteroaryl having 1 to 3 heteroatoms, wherein each heteroatom of the heteroaryl is independently selected from N, S, and O;
X is H, halo, C₁₋₃ alkyl optionally substituted with one or more halo, or a C₁₋₃ alkoxyl, wherein the alkoxyl and

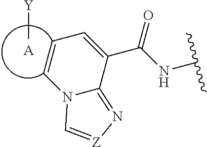

are meta- or para-relative to each other;
Y is H, C₁₋₄ alkyl, or C₃₋₄ cycloalkyl, wherein the C₁₋₄ alkyl is a primary or secondary alkyl; and
Z is CH or N,

a stereoisomer or mixture of stereoisomers thereof, or a pharmaceutically acceptable salt of each thereof.

Embodiment 2. The compound of embodiment 1, wherein the

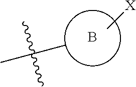

is

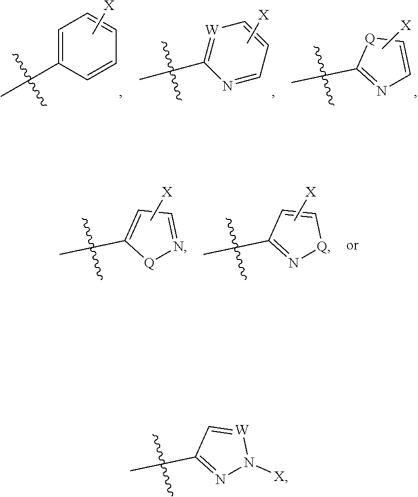

wherein Q is O or S, and W is CH or N,
a stereoisomer or mixture of stereoisomers thereof, or a pharmaceutically acceptable salt of each thereof.

Embodiment 3. The compound of embodiment 1, wherein

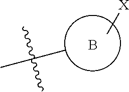

is selected from

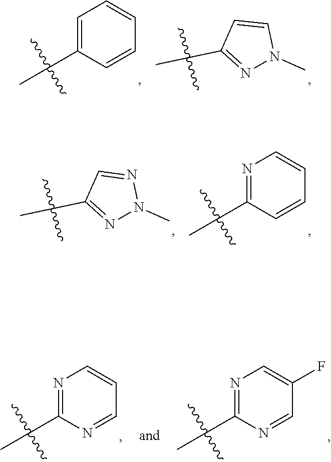

a stereoisomer or mixture of stereoisomers thereof, or a pharmaceutically acceptable salt of each thereof.

Embodiment 4. The compound of embodiment 1, having formula:

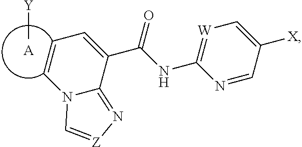

wherein:
X is H or halo;
Y is H or C₁₋₃ alkyl; and
W is CH or N,

a stereoisomer or mixture of stereoisomers thereof, or a pharmaceutically acceptable salt of each thereof.

Embodiment 5. The compound of any of embodiments 1-4, wherein

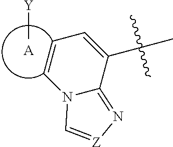

is selected from:

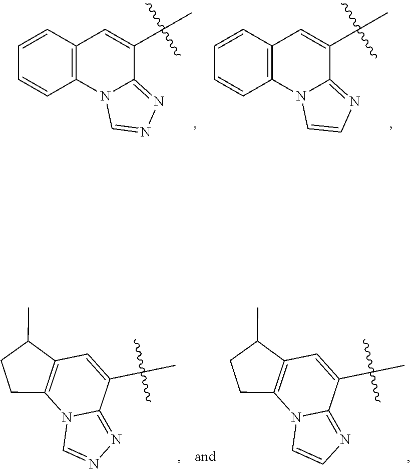 [SMILES: *c1cc2ccccc2n2cnnc12 ; *c1cc2ccccc2n2ccnc12 ; *c1cc2c(n3cnnc13)CCC2C ; *c1cc2c(n3ccnc13)CCC2C]

a stereoisomer or mixture of stereoisomers thereof, or a pharmaceutically acceptable salt of each thereof.

Embodiment 6. The compound of any of embodiments 1-4, wherein ring A is phenyl, or a pharmaceutically acceptable salt of each thereof.

Embodiment 7. The compound of any of embodiments 1-4, wherein ring A is

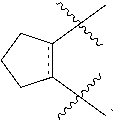

a stereoisomer or mixture of stereoisomers thereof, or a pharmaceutically acceptable salt of each thereof.

Embodiment 8. The compound of embodiment 1, having the formula:

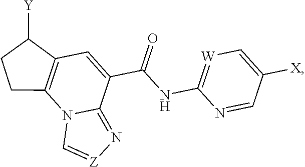

wherein:
X is H or halo;
Y is H or C₁₋₃ alkyl; and
W is CH or N,
a stereoisomer or mixture of stereoisomers thereof, or a pharmaceutically acceptable salt of each thereof.

Embodiment 9. The compound of any preceding embodiment, wherein Z is N, a stereoisomer or mixture of stereoisomers thereof, or a pharmaceutically acceptable salt of each thereof.

Embodiment 10. The compound of any of embodiments 1-8, wherein Z is CH, a stereoisomer or mixture of stereoisomers thereof, or a pharmaceutically acceptable salt of each thereof.

Embodiment 11. The compound of any of embodiments 1-10, wherein W is N, a stereoisomer or mixture of stereoisomers thereof, or a pharmaceutically acceptable salt of each thereof.

Embodiment 12. The compound of any of embodiments 1-11, wherein X is F, a stereoisomer or mixture of stereoisomers thereof, or a pharmaceutically acceptable salt of each thereof.

Embodiment 13. The compound of any of embodiments 1-4 and 6-12, wherein Y is methyl, a stereoisomer or mixture of stereoisomers thereof, or a pharmaceutically acceptable salt of each thereof.

Embodiment 14. A compound selected from the table below, a stereoisomer or mixture of stereoisomers thereof, or a pharmaceutically acceptable salt of each thereof:

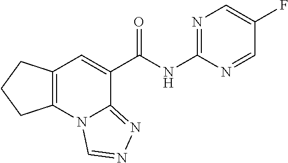 [SMILES: O=C(Nc1ncc(F)cn1)c1cc2c(n3cnnc13)CCC2]

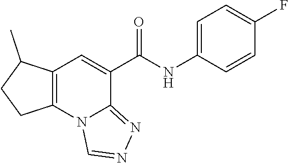 [SMILES: CC1CCc2c1cc(C(=O)Nc1ccc(F)cc1)c1nncn21]

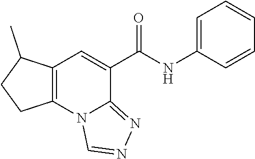 [SMILES: CC1CCc2c1cc(C(=O)Nc1ccccc1)c1nncn21]

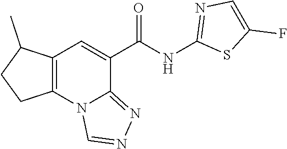 [SMILES: CC1CCc2c1cc(C(=O)Nc1ncc(F)s1)c1nncn21]

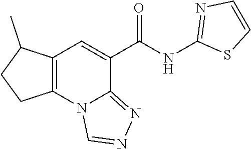 [SMILES: CC1CCc2c1cc(C(=O)Nc1nccs1)c1nncn21]

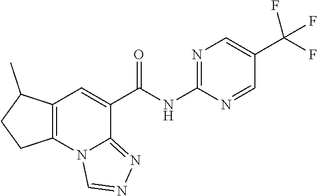 [SMILES: CC1CCc2c1cc(C(=O)Nc1ncc(C(F)(F)F)cn1)c1nncn21]

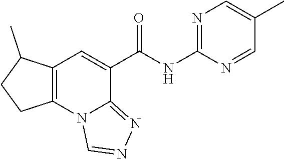 [SMILES: Cc1cnc(NC(=O)c2cc3c(n4cnnc24)CCC3C)nc1]

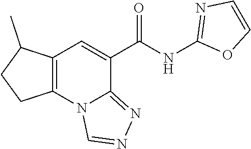 [SMILES: CC1CCc2c1cc(C(=O)Nc1ncco1)c1nncn21]

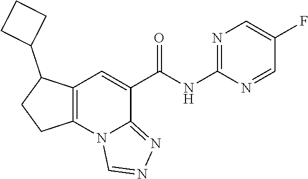 [SMILES: O=C(Nc1ncc(F)cn1)c1cc2c(n3cnnc13)CCC2C1CCC1]

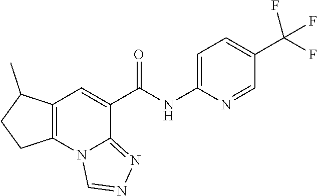 [SMILES: CC1CCc2c1cc(C(=O)Nc1ccc(C(F)(F)F)cn1)c1nncn21]

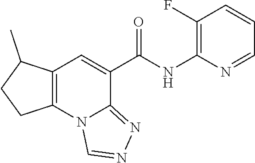 [SMILES: CC1CCc2c1cc(C(=O)Nc1ncccc1F)c1nncn21]

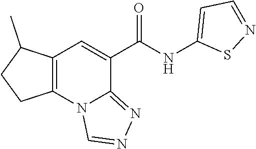 [SMILES: CC1CCc2c1cc(C(=O)Nc1ccns1)c1nncn21]

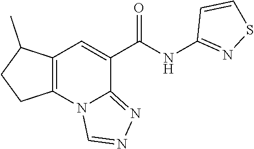 [SMILES: CC1CCc2c1cc(C(=O)Nc1ccsn1)c1nncn21]

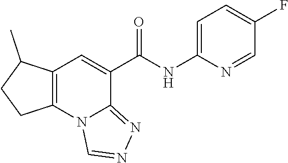 [SMILES: CC1CCc2c1cc(C(=O)Nc1ccc(F)cn1)c1nncn21]

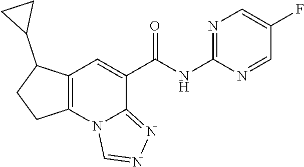 [SMILES: O=C(Nc1ncc(F)cn1)c1cc2c(n3cnnc13)CCC2C1CC1]

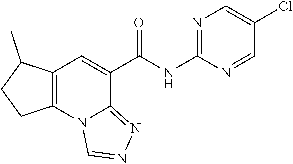 [SMILES: CC1CCc2c1cc(C(=O)Nc1ncc(Cl)cn1)c1nncn21]

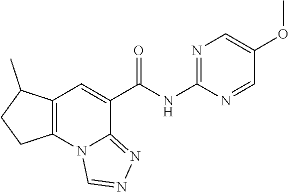 [SMILES: COc1cnc(NC(=O)c2cc3c(n4cnnc24)CCC3C)nc1]

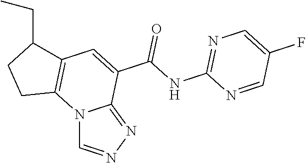 [SMILES: CCC1CCc2c1cc(C(=O)Nc1ncc(F)cn1)c1nncn21]

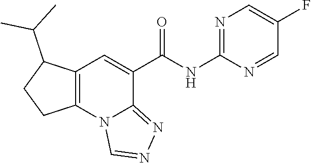 [SMILES: CC(C)C1CCc2c1cc(C(=O)Nc1ncc(F)cn1)c1nncn21]

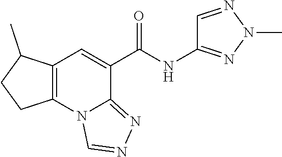 [SMILES: CC1CCc2c1cc(C(=O)Nc1cnn(C)n1)c1nncn21]

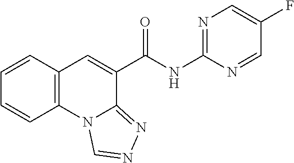 [SMILES: O=C(Nc1ncc(F)cn1)c1cc2ccccc2n2cnnc12]

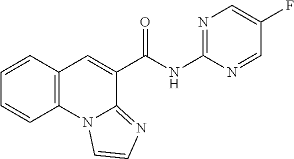 [SMILES: O=C(Nc1ncc(F)cn1)c1cc2ccccc2n2ccnc12]

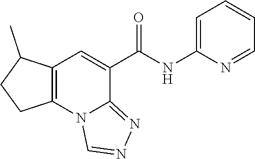 [SMILES: CC1CCc2c1cc(C(=O)Nc1ccccn1)c1nncn21]

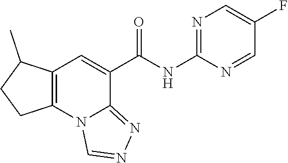 [SMILES: CC1CCc2c1cc(C(=O)Nc1ncc(F)cn1)c1nncn21]

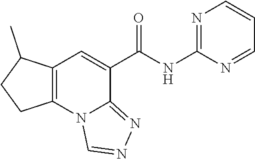 [SMILES: CC1CCc2c1cc(C(=O)Nc1ncccn1)c1nncn21]

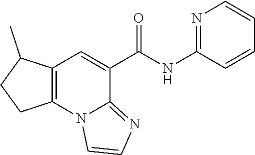 [SMILES: CC1CCc2c1cc(C(=O)Nc1ccccn1)c1nccn21]

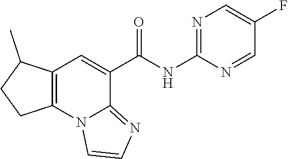 [SMILES: CC1CCc2c1cc(C(=O)Nc1ncc(F)cn1)c1nccn21]

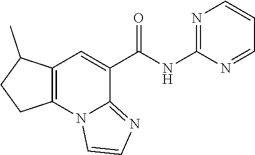 [SMILES: CC1CCc2c1cc(C(=O)Nc1ncccn1)c1nccn21]

Embodiment 14A. A compound selected from the table below, a stereoisomer or mixture of stereoisomers thereof, or a pharmaceutically acceptable salt of each thereof:

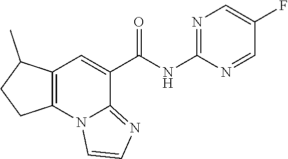 [SMILES: CC1CCc2c1cc(C(=O)Nc1ncc(F)cn1)c1nccn21]

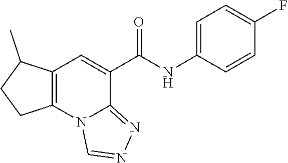 [SMILES: CC1CCc2c1cc(C(=O)Nc1ccc(F)cc1)c1nncn21]

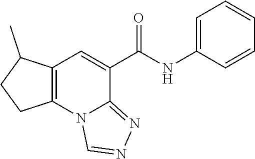 [SMILES: CC1CCc2c1cc(C(=O)Nc1ccccc1)c1nncn21]

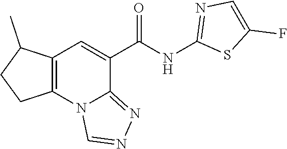 [SMILES: CC1CCc2c1cc(C(=O)Nc1ncc(F)s1)c1nncn21]

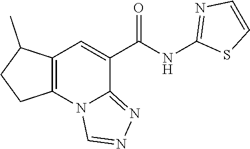 [SMILES: CC1CCc2c1cc(C(=O)Nc1nccs1)c1nncn21]

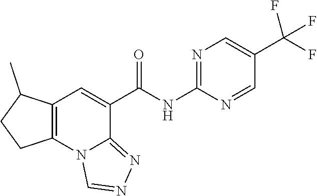 [SMILES: CC1CCc2c1cc(C(=O)Nc1ncc(C(F)(F)F)cn1)c1nncn21]

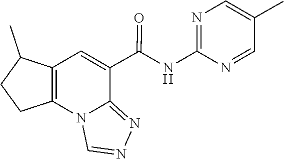 [SMILES: Cc1cnc(NC(=O)c2cc3c(n4cnnc24)CCC3C)nc1]

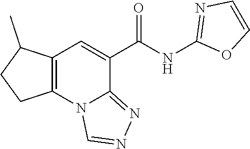 [SMILES: CC1CCc2c1cc(C(=O)Nc1ncco1)c1nncn21]

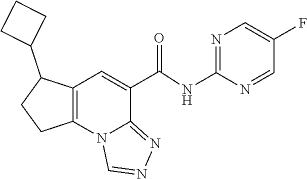 [SMILES: O=C(Nc1ncc(F)cn1)c1cc2c(n3cnnc13)CCC2C1CCC1]

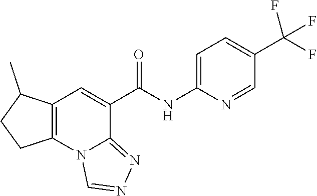 [SMILES: CC1CCc2c1cc(C(=O)Nc1ccc(C(F)(F)F)cn1)c1nncn21]

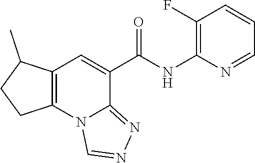 [SMILES: CC1CCc2c1cc(C(=O)Nc1ncccc1F)c1nncn21]

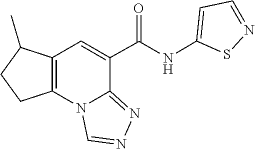 [SMILES: CC1CCc2c1cc(C(=O)Nc1ccns1)c1nncn21]

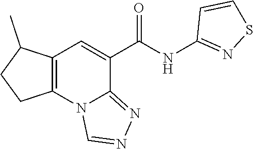 [SMILES: CC1CCc2c1cc(C(=O)Nc1ccsn1)c1nncn21]

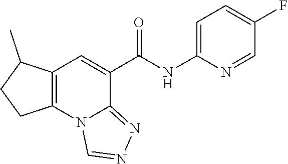 [SMILES: CC1CCc2c1cc(C(=O)Nc1ccc(F)cn1)c1nncn21]

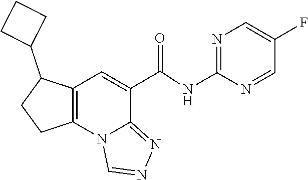 [SMILES: O=C(Nc1ncc(F)cn1)c1cc2c(n3cnnc13)CCC2C1CCC1]

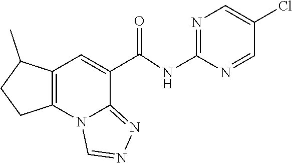 [SMILES: CC1CCc2c1cc(C(=O)Nc1ncc(Cl)cn1)c1nncn21]

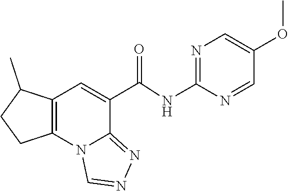 [SMILES: COc1cnc(NC(=O)c2cc3c(n4cnnc24)CCC3C)nc1]

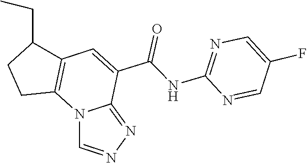 [SMILES: CCC1CCc2c1cc(C(=O)Nc1ncc(F)cn1)c1nncn21]

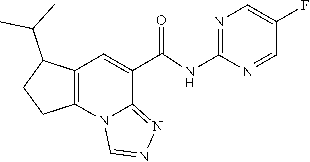 [SMILES: CC(C)C1CCc2c1cc(C(=O)Nc1ncc(F)cn1)c1nncn21]

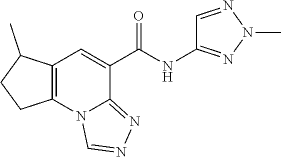 [SMILES: CC1CCc2c1cc(C(=O)Nc1cnn(C)n1)c1nncn21]

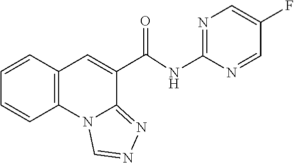 [SMILES: O=C(Nc1ncc(F)cn1)c1cc2ccccc2n2cnnc12]

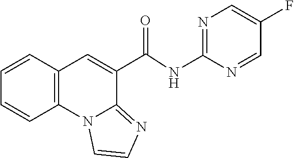 [SMILES: O=C(Nc1ncc(F)cn1)c1cc2ccccc2n2ccnc12]

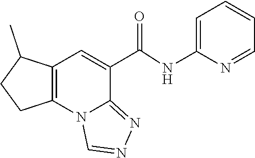 [SMILES: CC1CCc2c1cc(C(=O)Nc1ccccn1)c1nncn21]

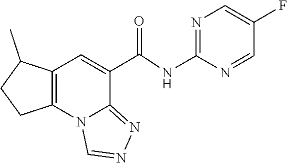 [SMILES: CC1CCc2c1cc(C(=O)Nc1ncc(F)cn1)c1nncn21]

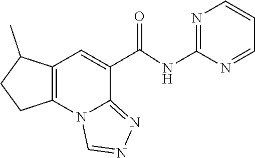 [SMILES: CC1CCc2c1cc(C(=O)Nc1ncccn1)c1nncn21]

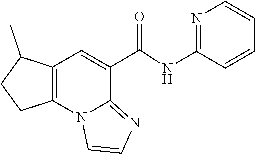 [SMILES: CC1CCc2c1cc(C(=O)Nc1ccccn1)c1nccn21]

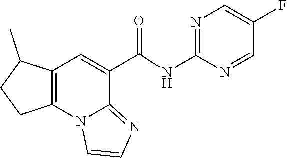 [SMILES: CC1CCc2c1cc(C(=O)Nc1ncc(F)cn1)c1nccn21]

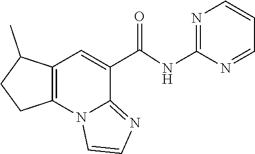 [SMILES: CC1CCc2c1cc(C(=O)Nc1ncccn1)c1nccn21]

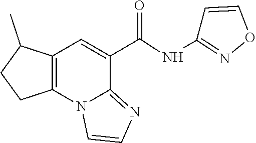 [SMILES: CC1CCc2c1cc(C(=O)Nc1ccon1)c1nccn21]

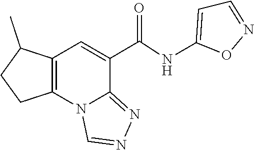 [SMILES: CC1CCc2c1cc(C(=O)Nc1ccno1)c1nncn21]

Embodiment 15. A compound selected from the following table, or a pharmaceutically acceptable salt thereof:

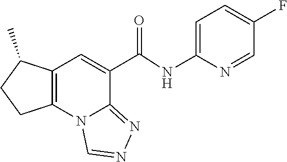 [SMILES: C[C@H]1CCc2c1cc(C(=O)Nc1ccc(F)cn1)c1nncn21]

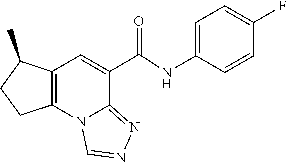 [SMILES: C[C@@H]1CCc2c1cc(C(=O)Nc1ccc(F)cc1)c1nncn21]

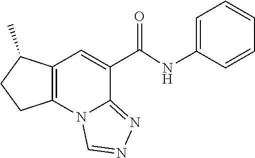 [SMILES: C[C@H]1CCc2c1cc(C(=O)Nc1ccccc1)c1nncn21]

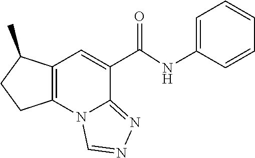 [SMILES: C[C@@H]1CCc2c1cc(C(=O)Nc1ccccc1)c1nncn21]

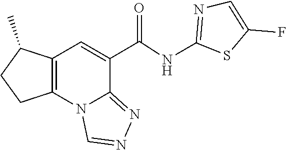 [SMILES: C[C@H]1CCc2c1cc(C(=O)Nc1ncc(F)s1)c1nncn21]

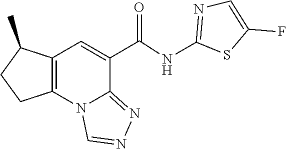 [SMILES: C[C@@H]1CCc2c1cc(C(=O)Nc1ncc(F)s1)c1nncn21]

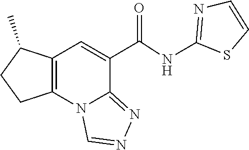 [SMILES: C[C@H]1CCc2c1cc(C(=O)Nc1nccs1)c1nncn21]

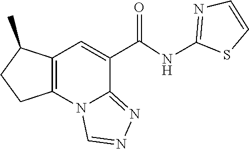 [SMILES: C[C@@H]1CCc2c1cc(C(=O)Nc1nccs1)c1nncn21]

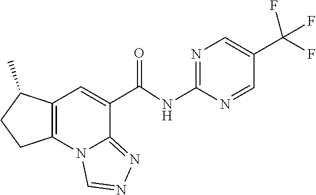 [SMILES: C[C@H]1CCc2c1cc(C(=O)Nc1ncc(C(F)(F)F)cn1)c1nncn21]

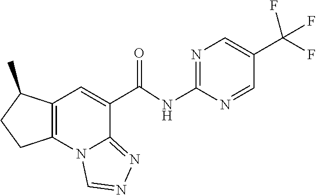 [SMILES: C[C@@H]1CCc2c1cc(C(=O)Nc1ncc(C(F)(F)F)cn1)c1nncn21]

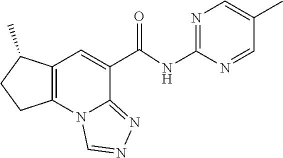 [SMILES: Cc1cnc(NC(=O)c2cc3c(n4cnnc24)CC[C@@H]3C)nc1]

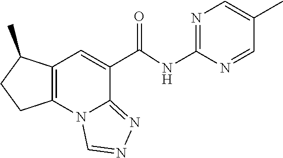 [SMILES: Cc1cnc(NC(=O)c2cc3c(n4cnnc24)CC[C@H]3C)nc1]

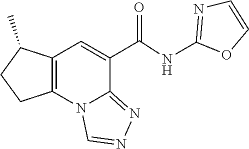 [SMILES: C[C@H]1CCc2c1cc(C(=O)Nc1ncco1)c1nncn21]

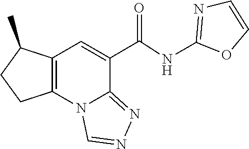 [SMILES: C[C@@H]1CCc2c1cc(C(=O)Nc1ncco1)c1nncn21]

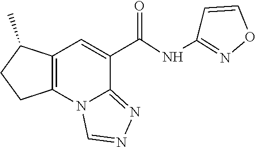 [SMILES: C[C@H]1CCc2c1cc(C(=O)Nc1ccon1)c1nncn21]

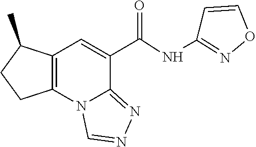 [SMILES: C[C@@H]1CCc2c1cc(C(=O)Nc1ccon1)c1nncn21]

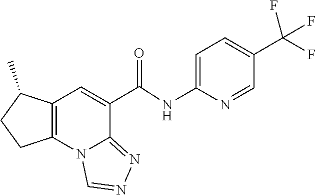 [SMILES: C[C@H]1CCc2c1cc(C(=O)Nc1ccc(C(F)(F)F)cn1)c1nncn21]

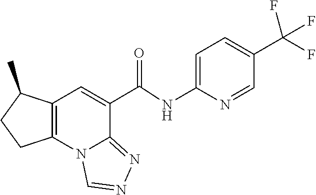 [SMILES: C[C@@H]1CCc2c1cc(C(=O)Nc1ccc(C(F)(F)F)cn1)c1nncn21]

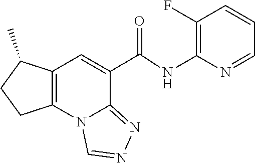 [SMILES: C[C@H]1CCc2c1cc(C(=O)Nc1ncccc1F)c1nncn21]

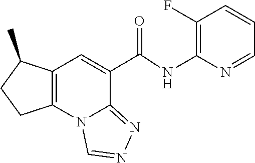 [SMILES: C[C@@H]1CCc2c1cc(C(=O)Nc1ncccc1F)c1nncn21]

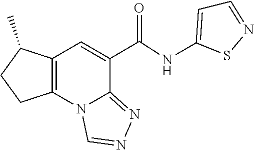 [SMILES: C[C@H]1CCc2c1cc(C(=O)Nc1ccns1)c1nncn21]

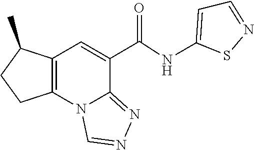 [SMILES: C[C@@H]1CCc2c1cc(C(=O)Nc1ccns1)c1nncn21]

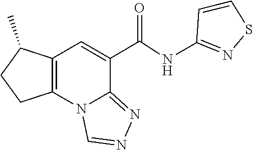 [SMILES: C[C@H]1CCc2c1cc(C(=O)Nc1ccsn1)c1nncn21]

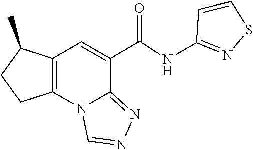 [SMILES: C[C@@H]1CCc2c1cc(C(=O)Nc1ccsn1)c1nncn21]

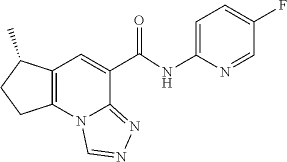 [SMILES: C[C@H]1CCc2c1cc(C(=O)Nc1ccc(F)cn1)c1nncn21]

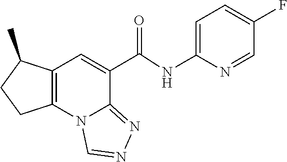 [SMILES: C[C@@H]1CCc2c1cc(C(=O)Nc1ccc(F)cn1)c1nncn21]

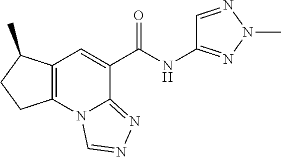 [SMILES: C[C@@H]1CCc2c1cc(C(=O)Nc1cnn(C)n1)c1nncn21]

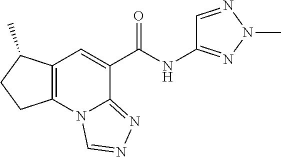 [SMILES: C[C@H]1CCc2c1cc(C(=O)Nc1cnn(C)n1)c1nncn21]

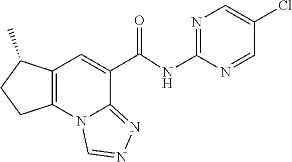 [SMILES: C[C@H]1CCc2c1cc(C(=O)Nc1ncc(Cl)cn1)c1nncn21]

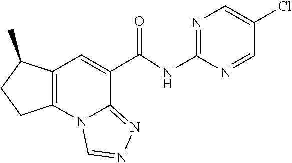 [SMILES: C[C@@H]1CCc2c1cc(C(=O)Nc1ncc(Cl)cn1)c1nncn21]

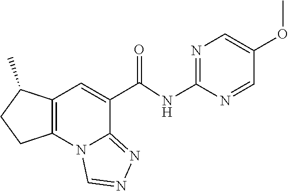 [SMILES: COc1cnc(NC(=O)c2cc3c(n4cnnc24)CC[C@@H]3C)nc1]

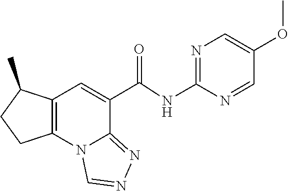 [SMILES: COc1cnc(NC(=O)c2cc3c(n4cnnc24)CC[C@H]3C)nc1]

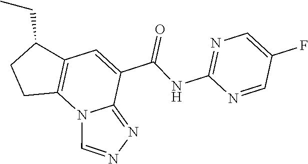 [SMILES: CC[C@H]1CCc2c1cc(C(=O)Nc1ncc(F)cn1)c1nncn21]

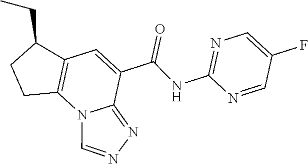 [SMILES: CC[C@@H]1CCc2c1cc(C(=O)Nc1ncc(F)cn1)c1nncn21]

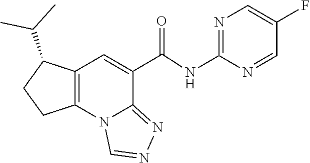 [SMILES: CC(C)[C@H]1CCc2c1cc(C(=O)Nc1ncc(F)cn1)c1nncn21]

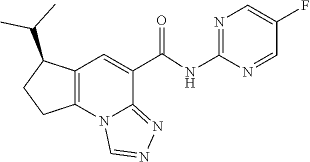 [SMILES: CC(C)[C@@H]1CCc2c1cc(C(=O)Nc1ncc(F)cn1)c1nncn21]

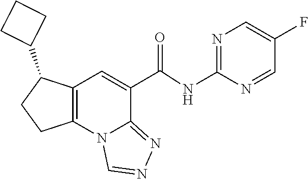 [SMILES: O=C(Nc1ncc(F)cn1)c1cc2c(n3cnnc13)CC[C@@H]2C1CCC1]

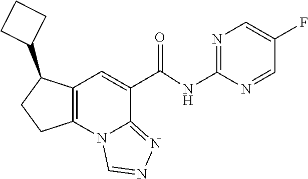 [SMILES: O=C(Nc1ncc(F)cn1)c1cc2c(n3cnnc13)CC[C@H]2C1CCC1]

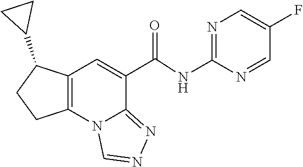 [SMILES: O=C(Nc1ncc(F)cn1)c1cc2c(n3cnnc13)CC[C@@H]2C1CC1]

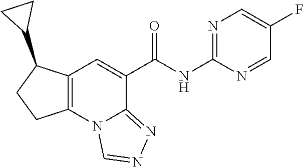 [SMILES: O=C(Nc1ncc(F)cn1)c1cc2c(n3cnnc13)CC[C@H]2C1CC1]

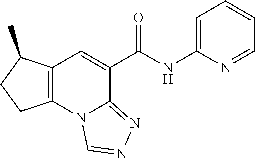 [SMILES: C[C@@H]1CCc2c1cc(C(=O)Nc1ccccn1)c1nncn21]

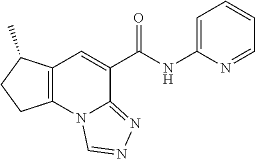 [SMILES: C[C@H]1CCc2c1cc(C(=O)Nc1ccccn1)c1nncn21]

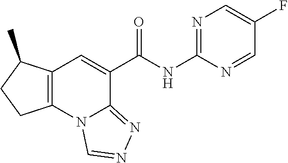 [SMILES: C[C@@H]1CCc2c1cc(C(=O)Nc1ncc(F)cn1)c1nncn21]

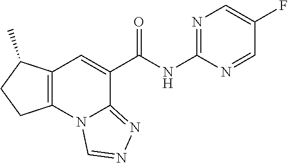 [SMILES: C[C@H]1CCc2c1cc(C(=O)Nc1ncc(F)cn1)c1nncn21]

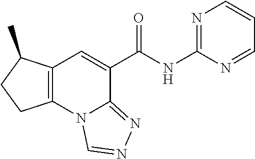 [SMILES: C[C@@H]1CCc2c1cc(C(=O)Nc1ncccn1)c1nncn21]

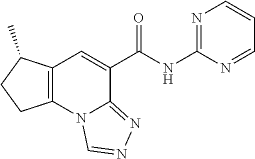 [SMILES: C[C@H]1CCc2c1cc(C(=O)Nc1ncccn1)c1nncn21]

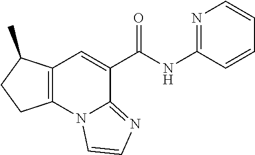 [SMILES: C[C@@H]1CCc2c1cc(C(=O)Nc1ccccn1)c1nccn21]

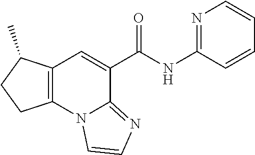 [SMILES: C[C@H]1CCc2c1cc(C(=O)Nc1ccccn1)c1nccn21]

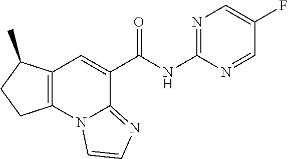 [SMILES: C[C@@H]1CCc2c1cc(C(=O)Nc1ncc(F)cn1)c1nccn21]

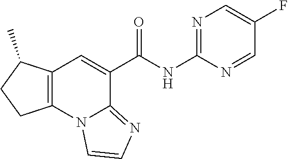 [SMILES: C[C@H]1CCc2c1cc(C(=O)Nc1ncc(F)cn1)c1nccn21]

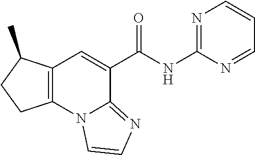 [SMILES: C[C@@H]1CCc2c1cc(C(=O)Nc1ncccn1)c1nccn21]

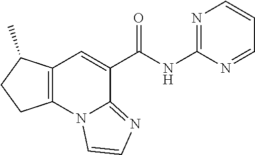 [SMILES: C[C@H]1CCc2c1cc(C(=O)Nc1ncccn1)c1nccn21]

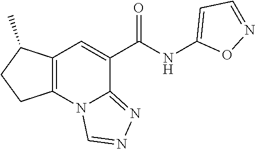 [SMILES: C[C@H]1CCc2c1cc(C(=O)Nc1ccno1)c1nncn21]

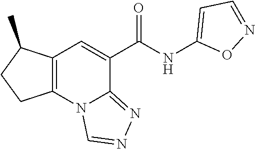 [SMILES: C[C@@H]1CCc2c1cc(C(=O)Nc1ccno1)c1nncn21]

Embodiment 15A. A compound selected from the following table, or a pharmaceutically acceptable salt thereof:

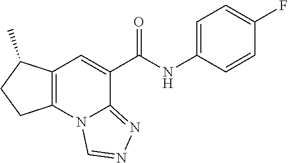 [SMILES: C[C@H]1CCc2c1cc(C(=O)Nc1ccc(F)cc1)c1nncn21]

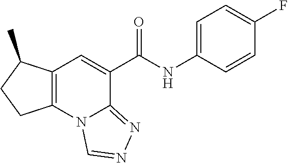 [SMILES: C[C@@H]1CCc2c1cc(C(=O)Nc1ccc(F)cc1)c1nncn21]

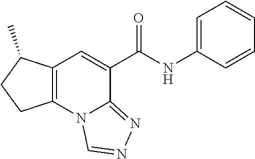 [SMILES: C[C@H]1CCc2c1cc(C(=O)Nc1ccccc1)c1nncn21]

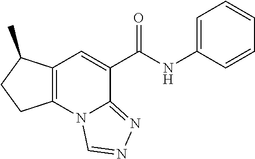 [SMILES: C[C@@H]1CCc2c1cc(C(=O)Nc1ccccc1)c1nncn21]

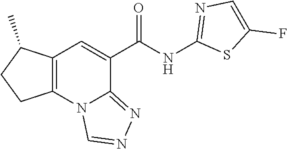 [SMILES: C[C@H]1CCc2c1cc(C(=O)Nc1ncc(F)s1)c1nncn21]

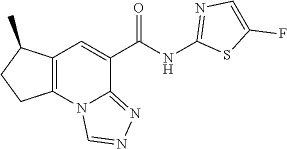 [SMILES: C[C@@H]1CCc2c1cc(C(=O)Nc1ncc(F)s1)c1nncn21]

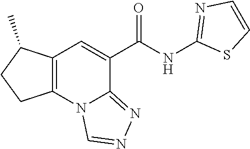 [SMILES: C[C@H]1CCc2c1cc(C(=O)Nc1nccs1)c1nncn21]

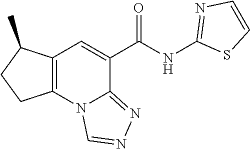 [SMILES: C[C@@H]1CCc2c1cc(C(=O)Nc1nccs1)c1nncn21]

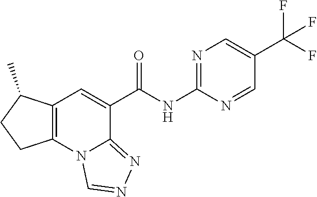 [SMILES: C[C@H]1CCc2c1cc(C(=O)Nc1ncc(C(F)(F)F)cn1)c1nncn21]

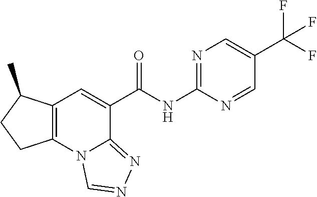 [SMILES: C[C@@H]1CCc2c1cc(C(=O)Nc1ncc(C(F)(F)F)cn1)c1nncn21]

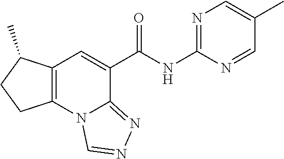 [SMILES: Cc1cnc(NC(=O)c2cc3c(n4cnnc24)CC[C@@H]3C)nc1]

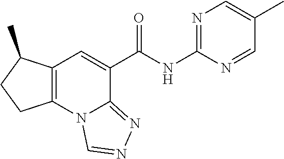 [SMILES: Cc1cnc(NC(=O)c2cc3c(n4cnnc24)CC[C@H]3C)nc1]

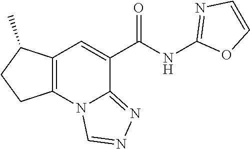 [SMILES: C[C@H]1CCc2c1cc(C(=O)Nc1ncco1)c1nncn21]

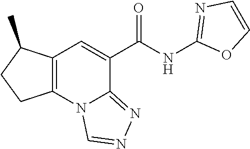 [SMILES: C[C@@H]1CCc2c1cc(C(=O)Nc1ncco1)c1nncn21]

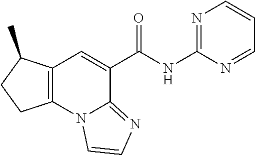 [SMILES: C[C@@H]1CCc2c1cc(C(=O)Nc1ncccn1)c1nccn21]

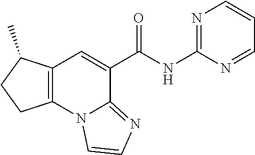 [SMILES: C[C@H]1CCc2c1cc(C(=O)Nc1ncccn1)c1nccn21]

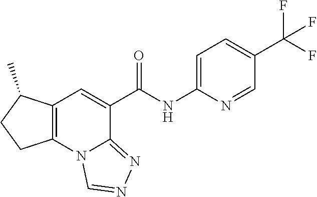 [SMILES: C[C@H]1CCc2c1cc(C(=O)Nc1ccc(C(F)(F)F)cn1)c1nncn21]

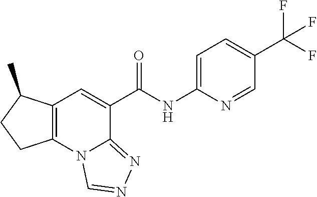 [SMILES: C[C@@H]1CCc2c1cc(C(=O)Nc1ccc(C(F)(F)F)cn1)c1nncn21]

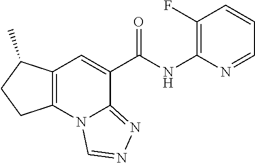 [SMILES: C[C@H]1CCc2c1cc(C(=O)Nc1ncccc1F)c1nncn21]

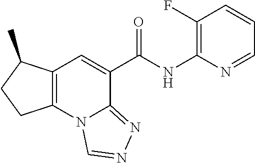 [SMILES: C[C@@H]1CCc2c1cc(C(=O)Nc1ncccc1F)c1nncn21]

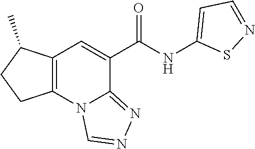 [SMILES: C[C@H]1CCc2c1cc(C(=O)Nc1ccns1)c1nncn21]

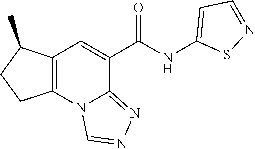 [SMILES: C[C@@H]1CCc2c1cc(C(=O)Nc1ccns1)c1nncn21]

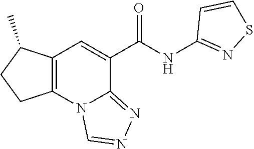 [SMILES: C[C@H]1CCc2c1cc(C(=O)Nc1ccsn1)c1nncn21]

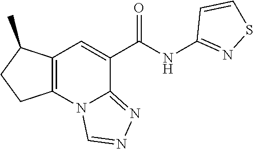 [SMILES: C[C@@H]1CCc2c1cc(C(=O)Nc1ccsn1)c1nncn21]

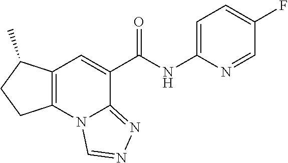 [SMILES: C[C@H]1CCc2c1cc(C(=O)Nc1ccc(F)cn1)c1nncn21]

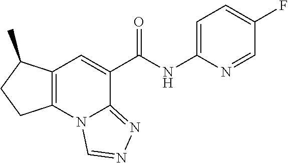 [SMILES: C[C@@H]1CCc2c1cc(C(=O)Nc1ccc(F)cn1)c1nncn21]

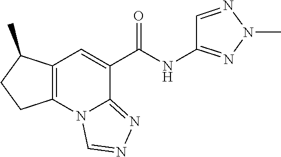 [SMILES: C[C@@H]1CCc2c1cc(C(=O)Nc1cnn(C)n1)c1nncn21]

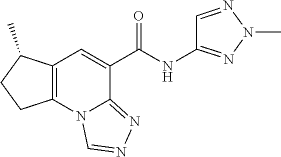 [SMILES: C[C@H]1CCc2c1cc(C(=O)Nc1cnn(C)n1)c1nncn21]

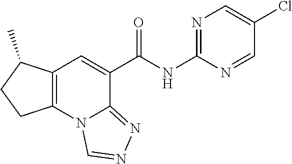 [SMILES: C[C@H]1CCc2c1cc(C(=O)Nc1ncc(Cl)cn1)c1nncn21]

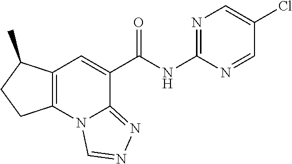 [SMILES: C[C@@H]1CCc2c1cc(C(=O)Nc1ncc(Cl)cn1)c1nncn21]

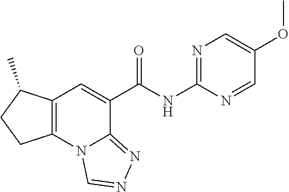 [SMILES: COc1cnc(NC(=O)c2cc3c(n4cnnc24)CC[C@@H]3C)nc1]

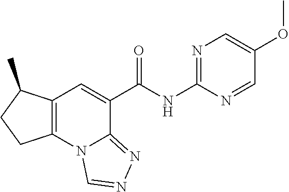 [SMILES: COc1cnc(NC(=O)c2cc3c(n4cnnc24)CC[C@H]3C)nc1]

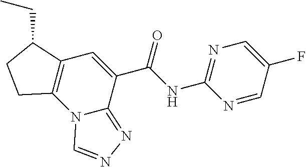 [SMILES: CC[C@H]1CCc2c1cc(C(=O)Nc1ncc(F)cn1)c1nncn21]

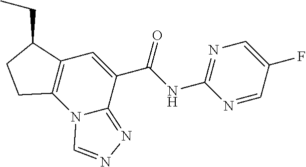 [SMILES: CC[C@@H]1CCc2c1cc(C(=O)Nc1ncc(F)cn1)c1nncn21]

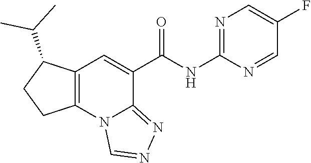 [SMILES: CC(C)[C@H]1CCc2c1cc(C(=O)Nc1ncc(F)cn1)c1nncn21]

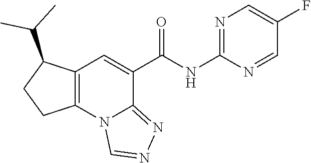 [SMILES: CC(C)[C@@H]1CCc2c1cc(C(=O)Nc1ncc(F)cn1)c1nncn21]

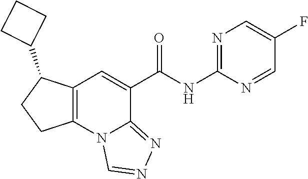 [SMILES: O=C(Nc1ncc(F)cn1)c1cc2c(n3cnnc13)CC[C@@H]2C1CCC1]

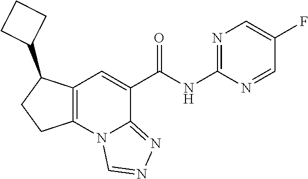 [SMILES: O=C(Nc1ncc(F)cn1)c1cc2c(n3cnnc13)CC[C@H]2C1CCC1]

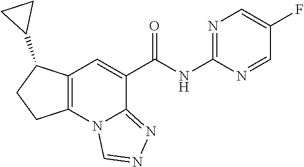 [SMILES: O=C(Nc1ncc(F)cn1)c1cc2c(n3cnnc13)CC[C@@H]2C1CC1]

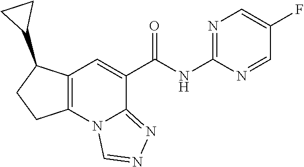 [SMILES: O=C(Nc1ncc(F)cn1)c1cc2c(n3cnnc13)CC[C@H]2C1CC1]

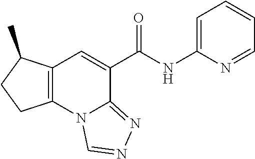 [SMILES: C[C@@H]1CCc2c1cc(C(=O)Nc1ccccn1)c1nncn21]

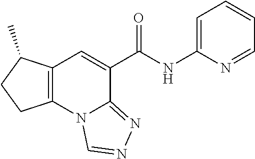 [SMILES: C[C@H]1CCc2c1cc(C(=O)Nc1ccccn1)c1nncn21]

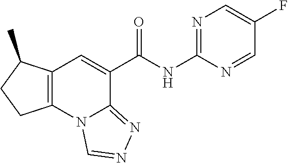 [SMILES: C[C@@H]1CCc2c1cc(C(=O)Nc1ncc(F)cn1)c1nncn21]

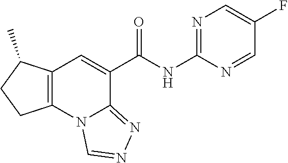 [SMILES: C[C@H]1CCc2c1cc(C(=O)Nc1ncc(F)cn1)c1nncn21]

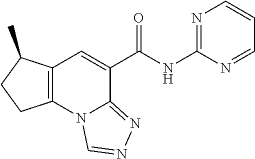 [SMILES: C[C@@H]1CCc2c1cc(C(=O)Nc1ncccn1)c1nncn21]

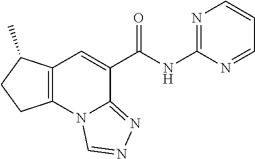 [SMILES: C[C@H]1CCc2c1cc(C(=O)Nc1ncccn1)c1nncn21]

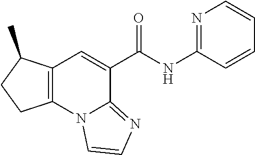 [SMILES: C[C@@H]1CCc2c1cc(C(=O)Nc1ccccn1)c1nccn21]

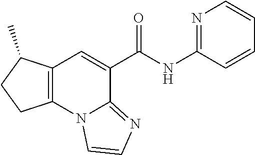 [SMILES: C[C@H]1CCc2c1cc(C(=O)Nc1ccccn1)c1nccn21]

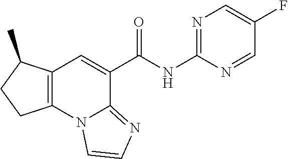 [SMILES: C[C@@H]1CCc2c1cc(C(=O)Nc1ncc(F)cn1)c1nccn21]

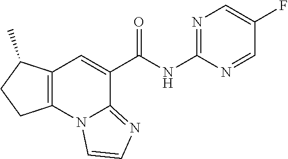 [SMILES: C[C@H]1CCc2c1cc(C(=O)Nc1ncc(F)cn1)c1nccn21]

Embodiment 16. A compound of

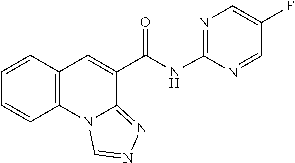 [SMILES: O=C(Nc1ncc(F)cn1)c1cc2ccccc2n2cnnc12]

or a pharmaceutically acceptable salt thereof.

Embodiment 17. A compound of

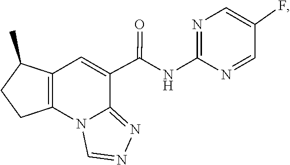 [SMILES: C[C@@H]1CCc2c1cc(C(=O)Nc1ncc(F)cn1)c1nncn21]

a stereoisomer or mixture of stereoisomers thereof, or a pharmaceutically acceptable salt of each thereof.

Embodiment 18. A compound of

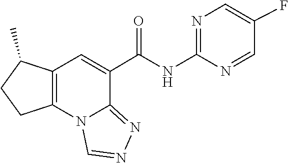 [SMILES: C[C@H]1CCc2c1cc(C(=O)Nc1ncc(F)cn1)c1nncn21]

or a pharmaceutically acceptable salt of each thereof.

Embodiment 19. A compound of

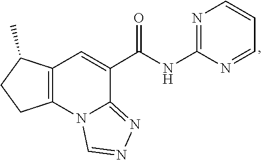 [SMILES: C[C@H]1CCc2c1cc(C(=O)Nc1ncccn1)c1nncn21]

a stereoisomer or mixture of stereoisomers thereof, or a pharmaceutically acceptable salt of each thereof.

Embodiment 20. A compound selected from Table 2, a stereoisomer or mixture of stereoisomers thereof, or a pharmaceutically acceptable salt of each thereof.

Embodiment 21. A pharmaceutical composition, comprising a compound of any of embodiments 1 to 20, a stereoisomer or mixture of stereoisomers thereof, or a pharmaceutically acceptable salt of each thereof, and one or more pharmaceutically acceptable carriers, diluents, or excipients.

Embodiment 22. A method of treating an immune-mediated disease in a patient in need thereof, comprising administering to the patient an effective amount of a compound according to any one of embodiments 1 to 20, a stereoisomer or mixture of stereoisomers thereof, or a pharmaceutically acceptable salt of each thereof, or the pharmaceutical composition according to embodiment 21.

Embodiment 23. A method of treating a disease or disorder selected from psoriasis, atopic dermatitis, ulcerative colitis, Crohn's disease, graft-versus-host disease, rheumatoid arthritis, and multiple sclerosis in a patient in need thereof, comprising administering to the patient an effective amount of a compound according to any one of embodiments 1 to 20, a stereoisomer or mixture of stereoisomers thereof, or a pharmaceutically acceptable salt of each thereof, or the pharmaceutical composition according to embodiment 21.

Embodiment 24. A compound according to any one of embodiments 1 to 20, a stereoisomer or mixture of stereoisomers thereof, or a pharmaceutically acceptable salt of each thereof, for use in therapy.

Embodiment 25. A compound according to any one of embodiments 1 to 20, a stereoisomer or mixture of stereoisomers thereof, or a pharmaceutically acceptable salt of each thereof, for use in treating a disease or disorder selected from psoriasis, atopic dermatitis, ulcerative colitis, Crohn's disease, graft-versus-host disease, rheumatoid arthritis, and multiple sclerosis.

Embodiment 26. A compound according to any one of embodiments 1 to 20, a stereoisomer or mixture of stereoisomers thereof, or a pharmaceutically acceptable salt thereof, for use in treating psoriasis.

Embodiment 27. A compound according to any one of embodiments 1 to 20, a stereoisomer or mixture of stereoisomers thereof, or a pharmaceutically acceptable salt thereof, for use in treating atopic dermatitis.

Embodiment 28. A compound according to any one of embodiments 1 to 20, a stereoisomer or mixture of stereoisomers thereof, or a pharmaceutically acceptable salt thereof, for use in treating ulcerative colitis.

Embodiment 29. A compound according to any one of embodiments 1 to 20, a stereoisomer or mixture of stereoisomers thereof, or a pharmaceutically acceptable salt thereof, for use in treating Crohn's disease.

Embodiment 30. A compound according to any one of embodiments 1 to 20, a stereoisomer or mixture of stereoisomers thereof, or a pharmaceutically acceptable salt thereof, for use in treating graft-versus-host disease.

Embodiment 31. A compound according to any one of embodiments 1 to 20, a stereoisomer or mixture of stereoisomers thereof, or a pharmaceutically acceptable salt thereof, for use in treating rheumatoid arthritis.

Embodiment 32. A compound according to any one of embodiments 1 to 20, a stereoisomer or mixture of stereoisomers thereof, or a pharmaceutically acceptable salt thereof, for use in treating multiple sclerosis.

Embodiment 33. A compound according to any one of embodiments 1 to 20, a stereoisomer or mixture of stereoisomers thereof, or a pharmaceutically acceptable salt thereof, for use in treating systemic lupus erythematosus (SLE).

Embodiment 34. A compound of the formula:

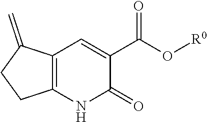

or a salt thereof, wherein R⁰ is C₁₋₃ alkyl.

Embodiment 35. A compound of the formula:

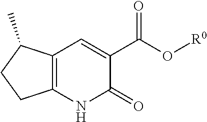

or a salt thereof, wherein R⁰ is C₁₋₃ alkyl.

Embodiment 36. A process for preparing a compound of embodiment 35, comprising contacting a compound of formula:

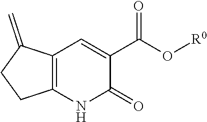

with molecular hydrogen under conditions sufficient to provide the compound of embodiment 35.

Embodiment 37. The process of embodiment 36, wherein the conditions include the presence of a chiral phosphine ligand.

Embodiment 37A. The process of embodiment 37, wherein the chiral phosphine ligand is a P-chiral phosphine ligand.

Embodiment 37B. The process of embodiment 37, wherein the chiral phosphine ligand is a P-stereogenic Cl-symmetric diphosphine ligand.

Embodiment 38. The process of embodiment 37, wherein the chiral phosphine ligand is a ChenPhos ligand.

Embodiment 38A. The process of claim 38, wherein the ChenPhos ligand includes two cyclohexyl groups bonded to P.

Embodiment 39. The process of embodiment 38, wherein the ChenPhos ligand is 1-(dicyclohexylphosphino)-1′-[(S)-[(1R)-2-[(1R)-1-(dimethylamino)ethyl]ferrocenyl]phenylphosphino]-Ferrocene.

Embodiment 40. The process of embodiment 37, wherein the conditions include the chiral phosphine ligand at a stoichiometric ratio of about 0.01:1 to about 0.05:1 relative to the compound of formula:

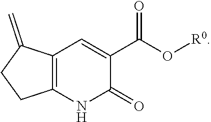

Embodiment 41. The process of any of embodiments 37 to 40, wherein the conditions include the presence of a catalyst of bis(norbornadiene)rhodium(I) salt.

Embodiment 42. The process of embodiment 41, wherein the conditions include the catalyst at a stoichiometric ratio of about 0.01:1 to about 0.05:1 relative to the compound of formula:

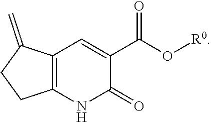

Embodiment 43. The process of embodiment 41, wherein the conditions include the chiral phosphine ligand at a stoichiometric ratio of about 1.2:1 to about 1:1 relative to the catalyst.

Embodiment 44. The process of any of embodiments 36-43, wherein the conditions include contacting under a substantially oxygen-free atmosphere.

Embodiment 45. The process of any of embodiments 36-44, wherein the conditions include contacting in a protic medium.

Embodiment 46. The process of any of embodiments 36-45, wherein the conditions include contacting at a temperature of about 10° C. to about 50° C.

Embodiment 47. The process of any of embodiments 36-46, wherein the conditions include molecular hydrogen at a pressure at about 0.5 MPa to about 2.5 MPa.

Embodiment 47A. The process of any of embodiments 36-46, wherein the conditions include molecular hydrogen at a pressure at about 1 MPa to about 2 MPa.

Embodiment 48. The process of any of embodiments 36-47, further comprising preparing the compound of formula:

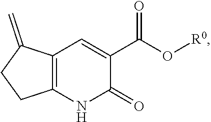

wherein the preparing includes contacting a compound of formula:

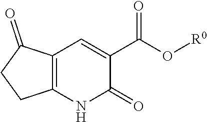

with a Wittig reagent.

Embodiment 49. The process of any of embodiments 36-48, further comprising preparing the compound of formula:

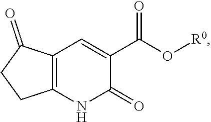

wherein the preparing includes contacting a compound of formula

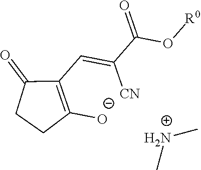

with an acid, such as an organic acid, at a temperature of about 70° C. to about 110° C.

Embodiment 50. The process of any of embodiments 36-49, further comprising preparing the compound of formula:

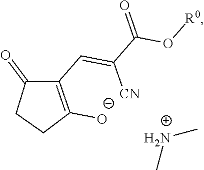

wherein the preparing includes contacting a compound of formula:

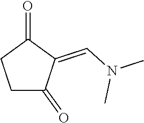

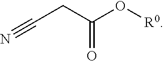

with a compound of formula:

Embodiment 51. A compound of formula:

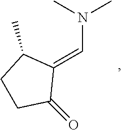

or a salt thereof.

Embodiment 52. A process of preparing a compound of formula:

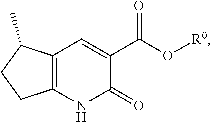

comprising contacting a compound of formula:

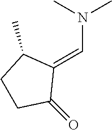

with a compound of formula:

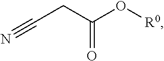

wherein R⁰ is C₁₋₃ alkyl.

Embodiment 53. A process of preparing a compound of formula:

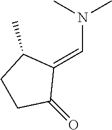

comprising contacting a racemic mixture of formula:

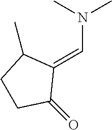

with a chiral acid under conditions sufficient to form a co-crystal.

Embodiment 53A. The process of embodiment 53, wherein the chiral acid is selected from D-(−)-tartaric acid, (+)-dibenzoyl-D-tartaric acid, di-p-toluoyl-L-tartaric acid, D-pyroglutamic acid, D-valine, L-isoleucine, L-histidine, N-acetyl-L-valine, D-proline, Naproxen, N-acetyl-L-phenylalanine, L-(+)-arginine(acid), D-(−)-quinic acid, (+)-deoxycholic acid, and N-acetyl-L-leucine.

Embodiment 53B. The process of embodiment 53, wherein the chiral acid is selected from (+)-dibenzoyl-D-tartaric acid, di-p-toluoyl-L-tartaric acid, D-pyroglutamic acid, L-isoleucine, L-histidine, D-proline, and N-acetyl-L-leucine at a condition sufficient to provide a co-crystal.

Embodiment 53C. The process of embodiment 53, wherein the chiral acid is (+)-dibenzoyl-D-tartaric acid.

Embodiment 53D. The process of embodiment 53, wherein the chiral acid is of formula:

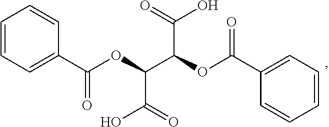 [SMILES: O=C(O[C@H](C(=O)O)[C@H](OC(=O)c1ccccc1)C(=O)O)c1ccccc1]

and the co-crystal is of formula:

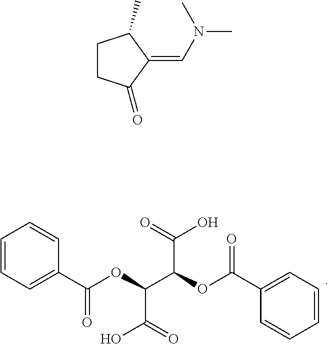 [SMILES: C[C@H]1CCC(=O)/C1=C/N(C)C ; O=C(O[C@H](C(=O)O)[C@H](OC(=O)c1ccccc1)C(=O)O)c1ccccc1]

Embodiment 53E. The process of embodiment 53, 53A, 53B, 53C, or 53D, wherein the conditions includes use of a solvent selected from: isopropanol, acetonitrile, isopropyl acetate, ethanol, butanone, and dimethoxyethane.

Embodiment 54. The process of embodiment 53, further comprising contacting the crystalline product with an aqueous basic solution.

Embodiment 54A. The process of embodiment 54, wherein the aqueous solution is sodium bicarbonate solution or potassium carbonate solution.

Embodiment 55. The process of embodiment 52, wherein the compound of formula:

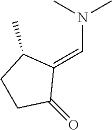

is prepared according to embodiment 53 or 54.

Embodiment 56. A process of preparing a compound of formula:

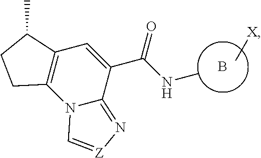

or a pharmaceutically acceptable salt thereof, comprising:
subjecting a compound of formula:

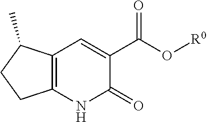

to conditions sufficient to provide a compound of formula:

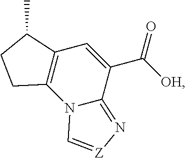

and
contacting the compound of formula:

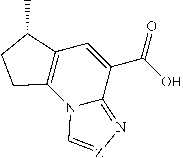

with a compound of formula:

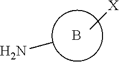

under conditions sufficient to provide the compound of formula:

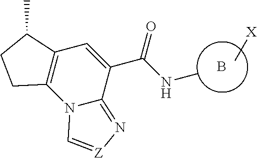

wherein:
ring B is a phenyl, or a 5-membered or 6-membered heteroaryl having 1 to 3 heteroatoms, wherein each heteroatom of the heteroaryl is independently selected from N, S, and O;
X is H, halo, C₁₋₃ alkyl optionally substituted with one or more halo, or a C₁₋₃ alkoxyl, wherein the alkoxyl and

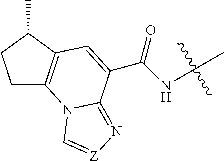

are meta- or para-relative to each other;
Z is CH or N; and
R⁰ is C₁₋₃ alkyl.

Embodiment 57. The process of embodiment 56, wherein the compound of formula:

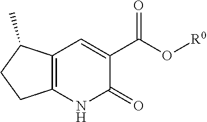

is prepared according to the process of embodiment 36, 52, or 55.

Embodiment 58. The process of embodiment 56, wherein Z is N, and wherein the condition that provides the compound of formula:

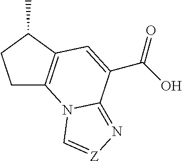

comprises:
contacting a compound of formula:

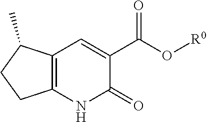

with phosphoryl chloride under conditions sufficient to provide a compound of formula:

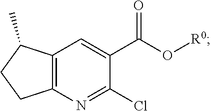

hydrolyzing the compound of formula:

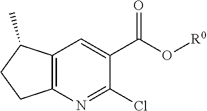

under conditions sufficient to provide a compound of formula:

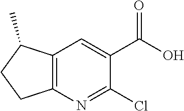

contacting a compound of formula

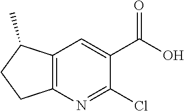

with hydrazine under conditions sufficient to provide a compound of formula:

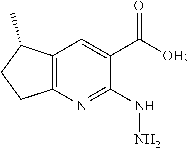

and
contacting the compound of formula:

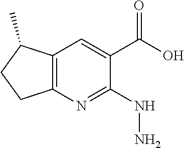

with formic acid under conditions sufficient to provide a compound of formula:

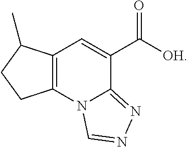 [SMILES: CC1CCc2c1cc(C(=O)O)c1nncn21]

Moreover, the present disclosure provides a compound of Formula I:

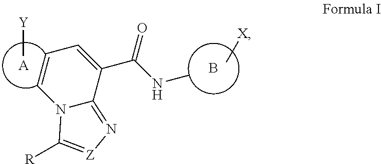

wherein:
ring A is a 5-membered or 6-membered carbocycle;
ring B is a phenyl, or a 5-membered or 6-membered heteroaryl having 1 or 2 heteroatoms, wherein each heteroatom of the heteroaryl is independently selected from N, S, and O;
R is H or C₁₋₃ alkyl;
X is H, halo, C₁₋₃ alkyl optionally substituted with one or more halo, or a C₁₋₃ alkoxyl;
Y is H, C₁₋₃ alkyl, or C₁₋₃ alkoxyl; and
Z is CH or N,
a stereoisomer or mixture of stereoisomers thereof, or a pharmaceutically acceptable salt of each thereof.

In some embodiments, ring A is phenyl and Y is H; or ring A is

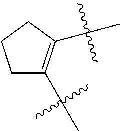 [SMILES: *C1=C(*)CCC1]

and Y is methyl; ring B is a pyridinyl or pyrimidinyl; R is H; X is H or F; and Z is CH or N, a stereoisomer or mixture of stereoisomers thereof, or a pharmaceutically acceptable salt of each thereof.

In some embodiments,

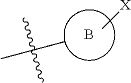

of the compound of Formula I is selected from the group consisting of phenyl,

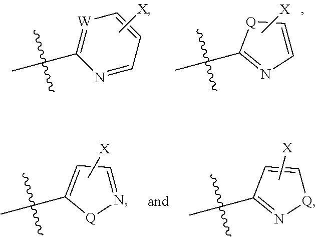

wherein Q is O or S, and W is CH or N.

In some embodiments, provided herein is a compound of Formula II:

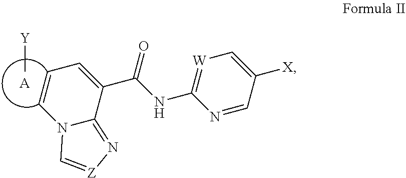

wherein X, Y, Z, and ring A are each as defined above with respect to Formula I, and

W is CH orN,

a stereoisomer or mixture of stereoisomers thereof, or a pharmaceutically acceptable salt of each thereof.

In some embodiments, ring A of the compound described in any preceding embodiment is phenyl. In some embodiments, ring A of the compound described in any preceding embodiment is

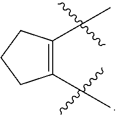 [SMILES: *C1=C(*)CCC1]

In some embodiments, provided here is a compound of Formula III:

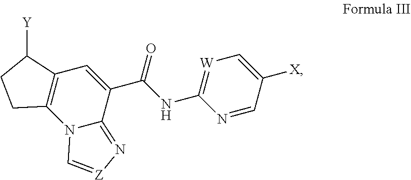

a stereoisomer or mixture of stereoisomers thereof, or a pharmaceutically acceptable salt of each thereof, wherein X, Y, Z, and W are as described above with respect to Formula I or Formula II.

In some embodiments, provided herein is a compound of Formula IV:

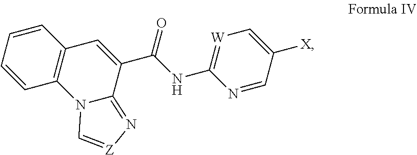

or a pharmaceutically acceptable salt thereof, wherein X, Y, Z, and W are as described above with respect to Formula I or Formula II.

In some embodiments, Z of the compound described in any preceding embodiment is N. In some embodiments, Z of the compound described in any preceding embodiment is CH.

In some embodiments, W of the compound described in any preceding embodiment is N.

In some embodiments, X of the compound described in any preceding embodiment is F.

In some embodiments, Y of the compound described in any preceding embodiment is methyl.

In some embodiments

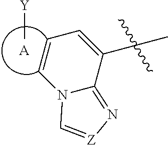

of the compound described in any preceding embodiment is selected from the group consisting of:

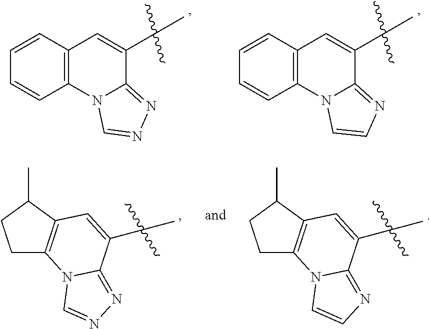 [SMILES: *c1cc2ccccc2n2cnnc12 ; *c1cc2ccccc2n2ccnc12 ; *c1cc2c(n3cnnc13)CCC2C ; *c1cc2c(n3ccnc13)CCC2C]

In some embodiments,

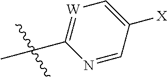

of the compound described in any preceding embodiment is selected from

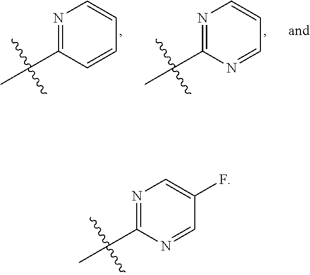 [SMILES: *c1ccccn1 ; *c1ncccn1 ; *c1ncc(F)cn1]

In some embodiments, the compound is as described in any preceding embodiment provided that when

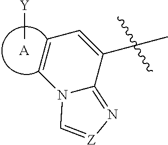

is

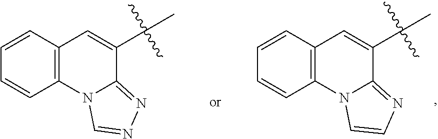 [SMILES: *c1cc2ccccc2n2cnnc12 ; "or" ; *c1cc2ccccc2n2ccnc12 ; ","]

then

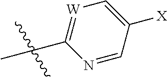

of the compound is

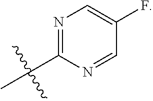 [SMILES: *c1ncc(F)cn1]

In some embodiments,

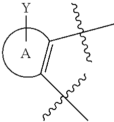

of the compound described in any preceding embodiment or

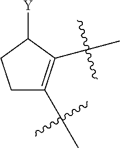

of the compound described in any preceding embodiment is

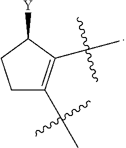

In some embodiments,

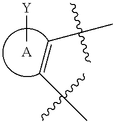

of the compound described in any preceding embodiment or

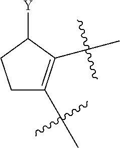

of the compound described in any preceding embodiment is

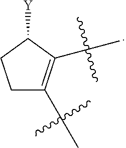

In some embodiments, provided here is a compound selected from the group consisting of:

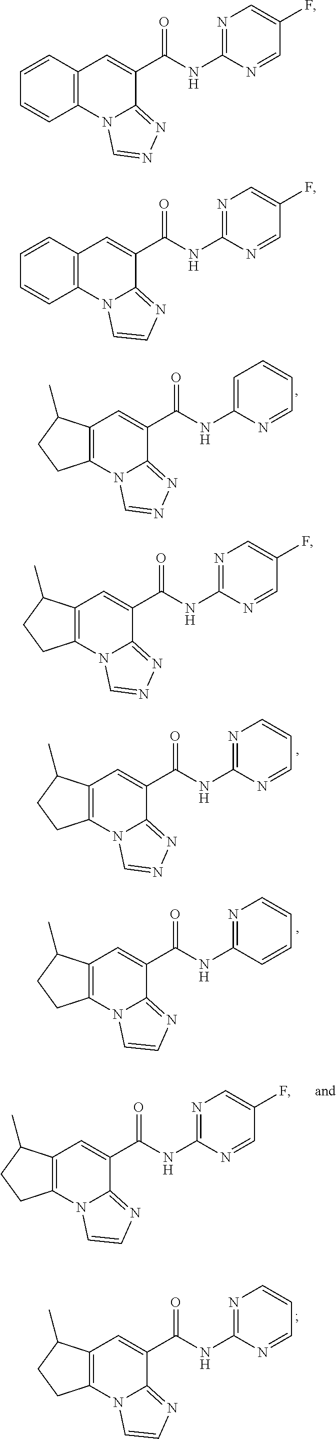

a stereoisomer or mixture of stereoisomers thereof, or a pharmaceutically acceptable salt of each thereof.

In some embodiments, provided here is a compound selected from the group consisting of:

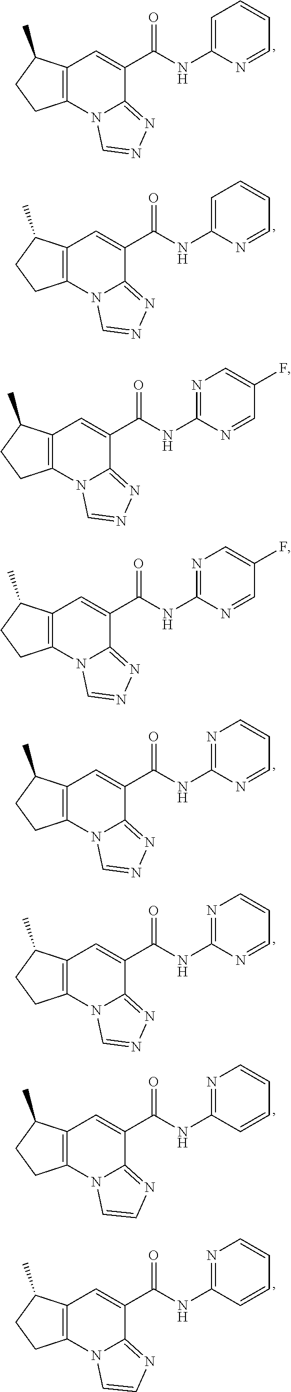

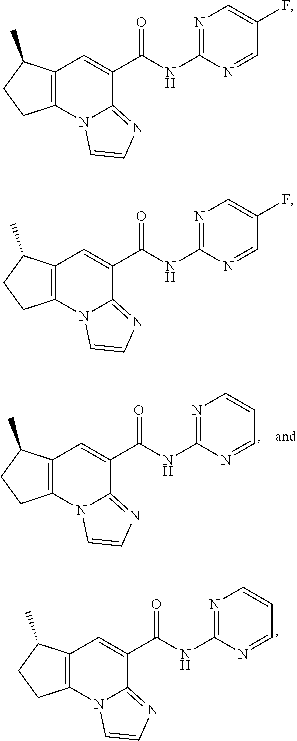 [SMILES: C[C@@H]1CCc2c1cc(C(=O)Nc1ncc(F)cn1)c1nccn21 ; C[C@H]1CCc2c1cc(C(=O)Nc1ncc(F)cn1)c1nccn21 ; C[C@@H]1CCc2c1cc(C(=O)Nc1ncccn1)c1nccn21 ; C[C@H]1CCc2c1cc(C(=O)Nc1ncccn1)c1nccn21]

a mixture of stereoisomers thereof, or a pharmaceutically acceptable salt of each thereof.

In some embodiments, provided here is a compound of

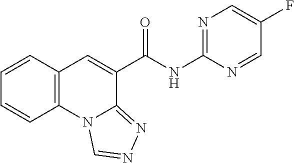 [SMILES: O=C(Nc1ncc(F)cn1)c1cc2ccccc2n2cnnc12]

or a pharmaceutically acceptable salt thereof.

In some embodiments, provided here is a compound of

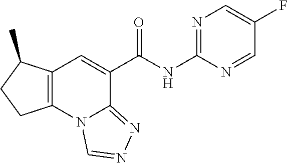 [SMILES: C[C@@H]1CCc2c1cc(C(=O)Nc1ncc(F)cn1)c1nncn21]

a stereoisomer or mixture of stereoisomers thereof, or a pharmaceutically acceptable salt of each thereof.

In some embodiments, provided here is a compound of

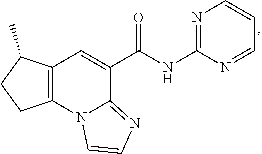 [SMILES: C[C@H]1CCc2c1cc(C(=O)Nc1ncccn1)c1nccn21]

a stereoisomer or mixture of stereoisomers thereof, or a pharmaceutically acceptable salt of each thereof.

The present disclosure further provides pharmaceutical composition, comprising a compound according to any of the above embodiments, a stereoisomer or mixture of stereoisomers thereof, or a pharmaceutically acceptable salt of each thereof, with one or more pharmaceutically acceptable carriers, diluents, or excipients.

The present disclosure provides a method of treating an immune-mediated disease in a patient, comprising administering to a patient in need of such treatment an effective amount of a compound, a stereoisomer or mixture of stereoisomers thereof, or a pharmaceutically acceptable salt of each thereof, or pharmaceutical composition according to any of the above embodiments.

The present disclosure also provides a method of treating a disease or disorder selected from psoriasis, atopic dermatitis, ulcerative colitis, Crohn's disease, graft-versus-host disease, rheumatoid arthritis, and multiple sclerosis in a patient, comprising administering to a patient in need of such treatment an effective amount of a compound, a stereoisomer or mixture of stereoisomers thereof, or a pharmaceutically acceptable salt of each thereof, or pharmaceutical composition according to any of the above embodiments.

The present disclosure provides a compound according to any of the above embodiments, a stereoisomer or mixture of stereoisomers thereof, or a pharmaceutically acceptable salt of each thereof, for use in therapy.

The present disclosure also provides a compound according to any of the above embodiments, a stereoisomer or mixture of stereoisomers thereof, or a pharmaceutically acceptable salt of each thereof, for use in the treatment of a disease or disorder selected from psoriasis, atopic dermatitis, ulcerative colitis, Crohn's disease, graft-versus-host disease, rheumatoid arthritis, and multiple sclerosis.

In some embodiments, the present disclosure provides a compound of Formula I-1:

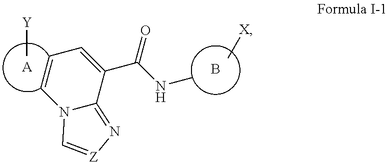

a stereoisomer or mixture of stereoisomers thereof, or a pharmaceutically acceptable salt of each thereof, wherein:
ring A is a 5-membered or 6-membered carbocycle;
ring B is a phenyl, or a 5-membered or 6-membered heteroaryl having 1 to 3 heteroatoms, wherein each heteroatom of the heteroaryl is independently selected from N, S, and O;
X is H, halo, C₁₋₃ alkyl optionally substituted with one or more halo, or a C₁₋₃ alkoxyl, wherein the alkoxyl is meta- or para-relative to the

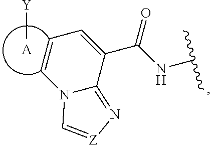

Y is H, C₁₋₄ alkyl, or C₃₋₄ cycloalkyl, wherein the C₁₋₄ alkyl is a primary or secondary alkyl; and
Z is CH or N.

In some embodiments, ring A is phenyl and Y is H; or ring A is

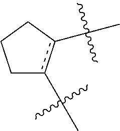

and Y is alkyl or cycloalkyl; ring B is a pyrazolyl, triazolyl, pyridinyl, or pyrimidinyl; X is H or F; and Z is CH or N, a stereoisomer or mixture of stereoisomers thereof, or a pharmaceutically acceptable salt of each thereof.

In some embodiments, provided herein is a compound of Formula I-2:

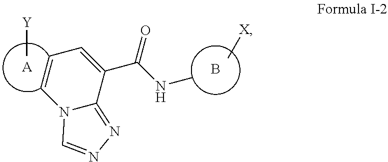

a stereoisomer or mixture of stereoisomers thereof, or a pharmaceutically acceptable salt of each thereof, wherein X, Y, Z, ring A, and ring B are each as defined above with respect to Formula I-1.

In some embodiments, provided herein is a compound of Formula I-3:

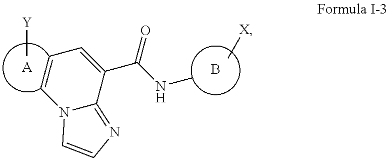

a stereoisomer or mixture of stereoisomers thereof, or a pharmaceutically acceptable salt of each thereof, wherein X, Y, Z, ring A, and ring B are each as defined above with respect to Formula I-1.

In some embodiments,

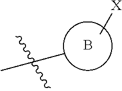

of the compound of Formula I-1, I-2, or I-3 is

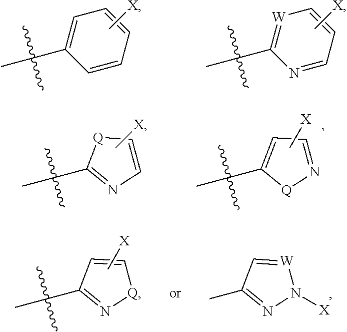

wherein Q is O or S, and W is CH or N.

In some embodiments,

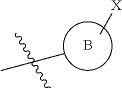

of the compound of Formula I-1, I-2, or I-3 is

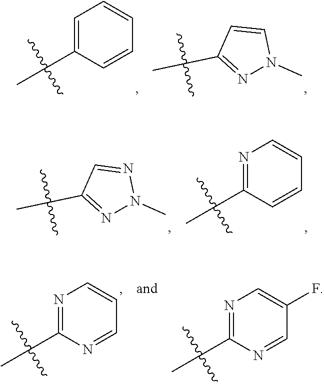

In some embodiments,

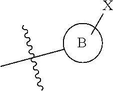

of the compound described in any preceding embodiment is a substituted or unsubstituted 6-member heteroaryl.

In some embodiments,

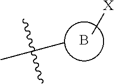

of the compound described in any preceding embodiment is a substituted or unsubstituted 5-member heteroaryl.

In some embodiments,

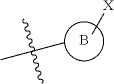

of the compound described in any preceding embodiment is

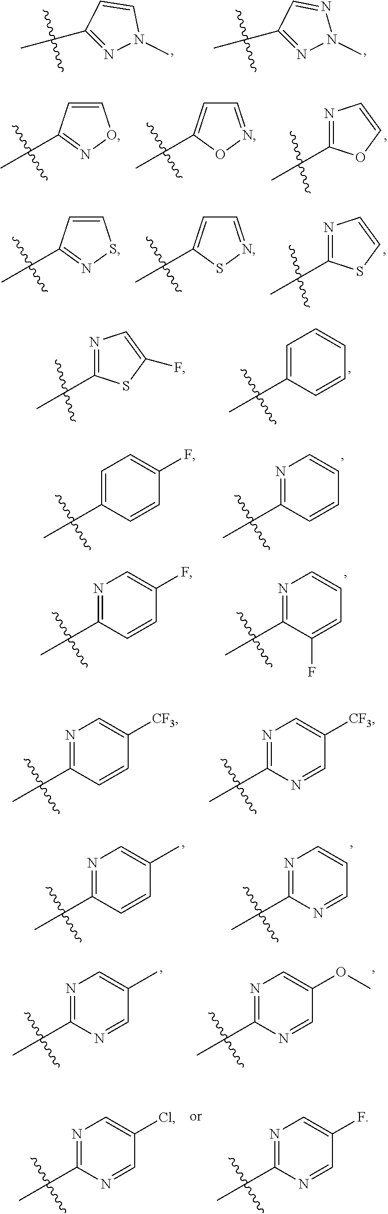

In some embodiments,

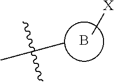

of the compound described in any preceding embodiment is

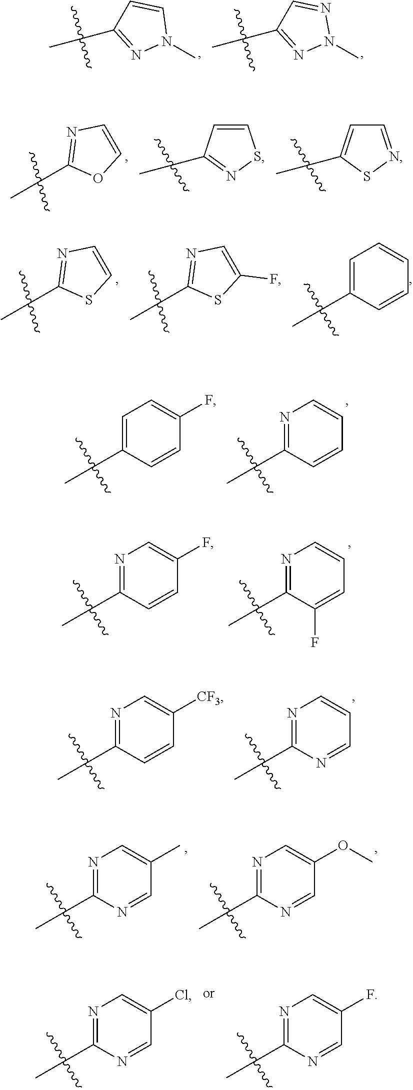

In some embodiments, provided herein is a compound of Formula II-1:

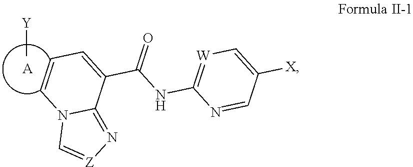

a stereoisomer or mixture of stereoisomers thereof, or a pharmaceutically acceptable salt of each thereof, wherein X, Y, Z, and ring A are each as defined above with respect to Formula I-1, and W is CH or N.

In some embodiments, provided herein is a compound of Formula II-2:

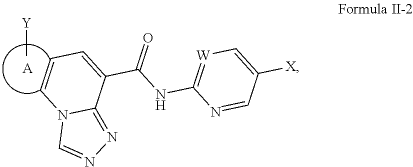

a stereoisomer or mixture of stereoisomers thereof, or a pharmaceutically acceptable salt of each thereof, wherein X, Y, and ring A are each as defined above with respect to Formula I-1, and W is CH or N.

In some embodiments, provided herein is a compound of Formula II-3:

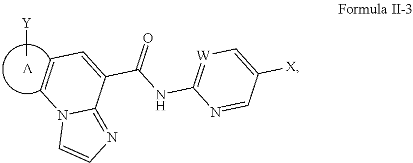

a stereoisomer or mixture of stereoisomers thereof, or a pharmaceutically acceptable salt of each thereof, wherein X, Y, and ring A are each as defined above with respect to Formula I-1, and W is CH or N.

In some embodiments, provided herein is a compound of Formula II-1A:

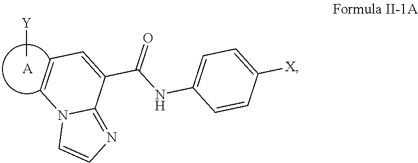

a stereoisomer or mixture of stereoisomers thereof, or a pharmaceutically acceptable salt of each thereof, wherein X, Y, and ring A are each as defined above with respect to Formula I-1.

In some embodiments, with respect to compounds of Formula II-1, II-2, II-3, and II-1A, wherein X is H or halo, and Y is H or C₁₋₃ alkyl.

In some embodiments,

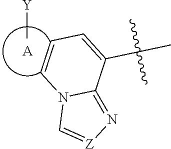

of the compound described in any preceding embodiment is selected from:

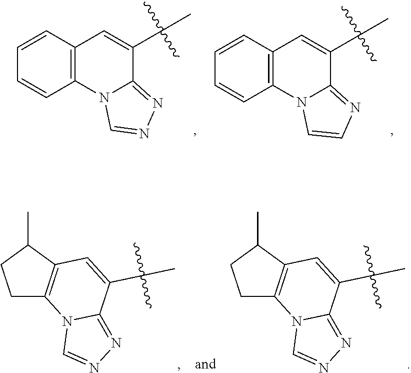 [SMILES: *c1cc2ccccc2n2cnnc12 ; *c1cc2ccccc2n2ccnc12 ; *c1cc2c(n3cnnc13)CCC2C ; *c1cc2c(n3cnnc13)CCC2C]

In some embodiments,

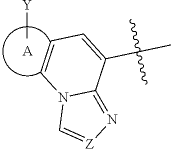

of the compound described in any preceding embodiment is selected from:

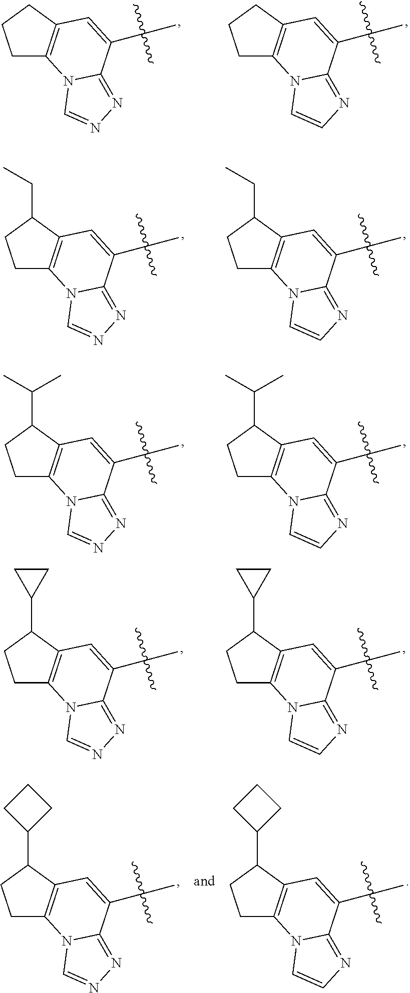

In some embodiments, the compound is as described in any preceding embodiment provided that when

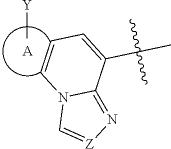

is

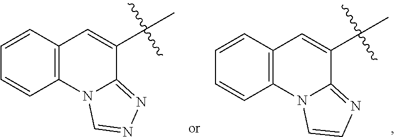 [SMILES: *c1cc2ccccc2n2cnnc12 ; *c1cc2ccccc2n2ccnc12]

then

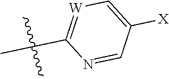

of the compound is

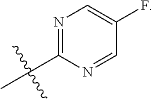 [SMILES: *c1ncc(F)cn1]

In some embodiments, ring A of the compound described in any preceding embodiment is phenyl. In some embodiments, ring A of the compound described in any preceding embodiment is

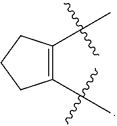 [SMILES: *C1=C(*)CCC1]

In some embodiments, provided here is a compound of Formula III-1:

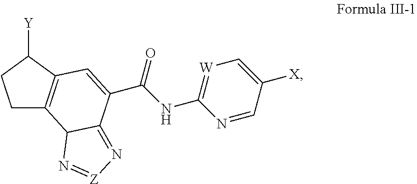

a stereoisomer or mixture of stereoisomers thereof, or a pharmaceutically acceptable salt of each thereof, wherein X, Y, and Z are as described above with respect to Formula I-1, wherein W is CH or N.

In some embodiments, provided here is a compound of Formula III-2:

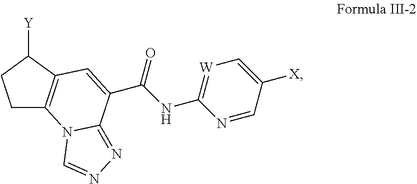

a stereoisomer or mixture of stereoisomers thereof, or a pharmaceutically acceptable salt of each thereof, wherein X and Y are as described above with respect to Formula I-1, wherein W is CH or N.

In some embodiments, provided here is a compound of Formula III-3:

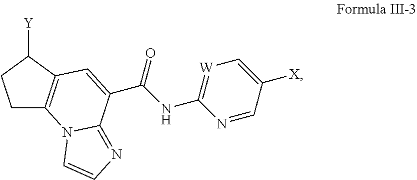

a stereoisomer or mixture of stereoisomers thereof, or a pharmaceutically acceptable salt of each thereof, wherein X and Y are as described above with respect to Formula I-1, wherein W is CH or N.

In some embodiments, ring B is a phenyl.

In some embodiments, provided here is a compound of Formula III-1A:

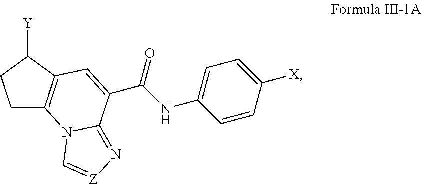

a stereoisomer or mixture of stereoisomers thereof, or a pharmaceutically acceptable salt of each thereof, wherein X, Y, and Z are as described above with respect to Formula I-1.

In some embodiments, provided herein is a compound of Formula IV-1:

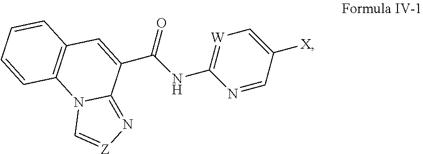

or a pharmaceutically acceptable salt thereof, wherein X and Z are as described above with respect to Formula I-1, wherein W is CH or N.

In some embodiments, provided herein is a compound of Formula IV-2:

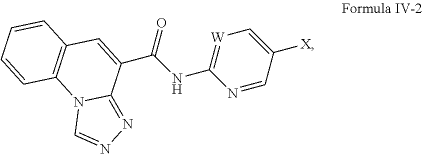

or a pharmaceutically acceptable salt thereof, wherein X is as described above with respect to Formula I-1, wherein W is CH or N.

In some embodiments, provided herein is a compound of Formula IV-3:

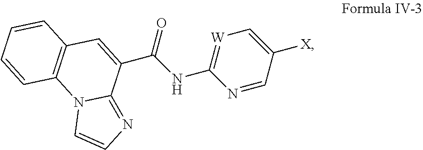

or a pharmaceutically acceptable salt thereof, wherein X is as described above with respect to Formula I-1, wherein W is CH or N.

In some embodiments, provided herein is a compound of Formula IV-1A:

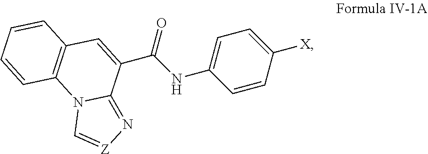

or a pharmaceutically acceptable salt thereof, wherein X and Z are as described above with respect to Formula I-1.

In some embodiments,

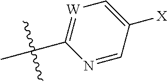

of the compound of formula II-1, 11-2, 11-3, III-1, III-2, III-3, IV-1, IV-2, or IV-3 is

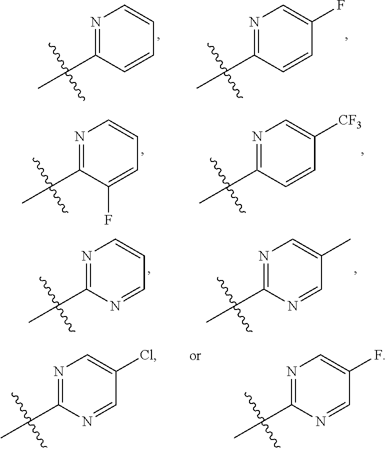

In some embodiments, provided here is a compound of Formula V-1:

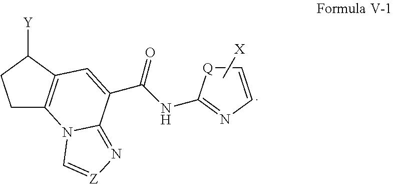

a stereoisomer or mixture of stereoisomers thereof, or a pharmaceutically acceptable salt of each thereof, wherein X, Y, and Z are as described above with respect to Formula I-1, and Q is O or S.

In some embodiments, provided here is a compound of Formula V-2:

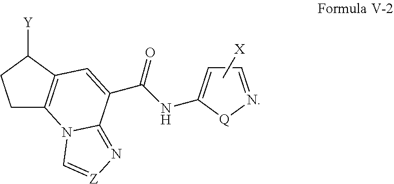

a stereoisomer or mixture of stereoisomers thereof, or a pharmaceutically acceptable salt of each thereof, wherein X, Y, and Z are as described above with respect to Formula I-1, and Q is 0 or S.

In some embodiments, provided here is a compound of Formula V-3:

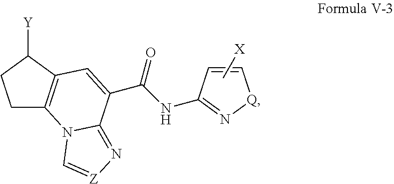

a stereoisomer or mixture of stereoisomers thereof, or a pharmaceutically acceptable salt of each thereof, wherein X, Y, and Z are as described above with respect to Formula I-1, and Q is 0 or S.

In some embodiments, provided herein is a compound of Formula V-4:

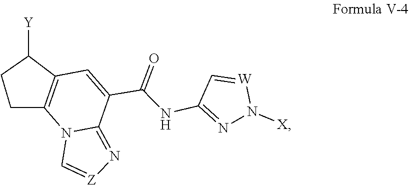

a stereoisomer or mixture of stereoisomers thereof, or a pharmaceutically acceptable salt of each thereof, wherein X, Y, and Z are as described above with respect to Formula I-1, and W is CH or N.

In some embodiments, with respect to any of compounds of Formula V-1, V-2, V-3, or V-4, wherein Z is N.

In some embodiments, with respect to any of compounds of Formula I-1, I-2, I-3, II-1, I-2, II-3, I-A, III-1, III-2, III-3, III-1A, IV-1, IV-2, IV-3, IV-A, V-1, V-2, V-3, and V-4, X is H or halo. In some embodiments, X is H or F. In some embodiments, X is F. In some embodiments, X is H. In some embodiments, X is Cl.

In some embodiments, with respect to any of compounds of Formula I-1, I-2, I-3, II-1, II-2, II-3, II-1A, III-1, III-2, III-3, III-1A, IV-1, IV-2, IV-3, IV-1A, V-1, V-2, V-3, and V-4, Y is H. In some embodiments, Y is C₁₋₄ alkyl. In some embodiments, Y is methyl. In some embodiments, Y is ethyl. In some embodiments, Y is propyl. In some embodiments, Y is isopropyl. In some embodiments, Y is butyl. In some embodiments, Y is sec-butyl. In some embodiments, Y is C₃₋₄ cycloalkyl. In some embodiments, Y of the compound described in any preceding embodiment is cyclopropyl. In some embodiments, Y of the compound described in any preceding embodiment is cyclobutyl.

In some embodiments, with respect to a compound of any preceding embodiment, X is F; and Y is C₃₋₄ cycloalkyl.

In some embodiments, with respect to a compound of any preceding embodiment, X is methyl.

In some embodiments, with respect to a compound of any preceding embodiment, X is C₁₋₃ alkyl substituted with one or more halo.

In some embodiments, with respect to a compound of any preceding embodiment, X is —CF₃.

In some embodiments, with respect to a compound of any preceding embodiment,

X is C₁₋₃ alkoxy, wherein the alkoxyl and

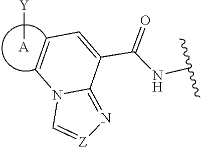

are meta-relative to each other.

In some embodiments, with respect to a compound of any preceding embodiment, X is C₁₋₃ alkoxy, wherein the alkoxyl and

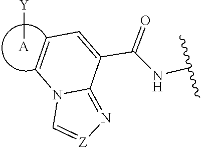

are para-relative to each other.

In some embodiments, with respect to a compound of any preceding embodiment, X is —OCH₃, wherein the —OCH₃ and

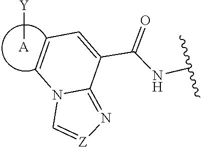

are meta-relative to each other.

In some embodiments, with respect to a compound of any preceding embodiment, X is —OCH₃, wherein the —OCH₃ and

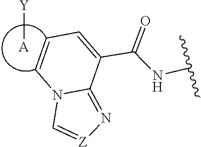

are para-relative to each other.

In some embodiments, W of the compound described in any of preceding embodiments II-1, II-2, II-3, III-1, III-2, III-3, IV-1, IV-2, and IV-3 is N.

In some embodiments, the

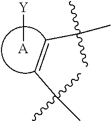

of the compound of formula I-1, I-2, I-3, II-1, II-2, or II-3, or the

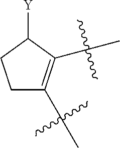

of the compound of formula III-1, III-2, or III-3 is

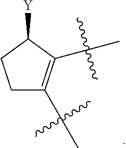

In some embodiments, the

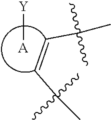

of the compound of formula I-1, I-2, I-3, II-1, II-2, or II-3, or the

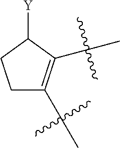

of the compound of formula III-1, III-2, or III-3 is

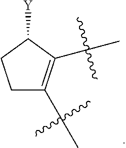

In some embodiments, provided here is a compound selected from:

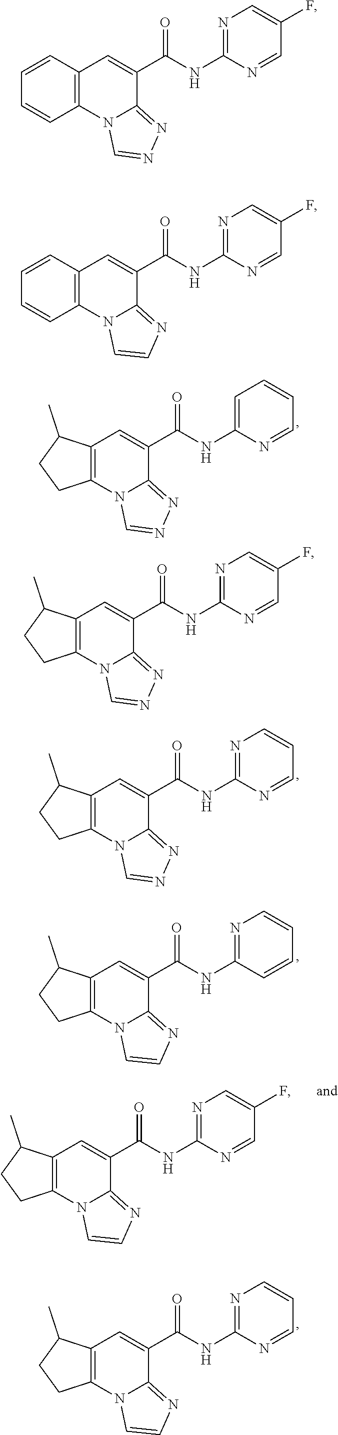

a stereoisomer or mixture of stereoisomers thereof, or a pharmaceutically acceptable salt of each thereof.

In some embodiments, provided here is a compound selected from the following table, a stereoisomer or mixture of stereoisomers thereof, or a pharmaceutically acceptable salt of each thereof:

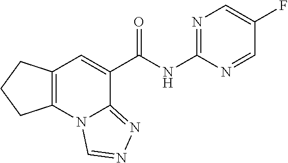 [SMILES: O=C(Nc1ncc(F)cn1)c1cc2c(n3cnnc13)CCC2]

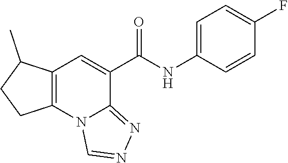 [SMILES: CC1CCc2c1cc(C(=O)Nc1ccc(F)cc1)c1nncn21]

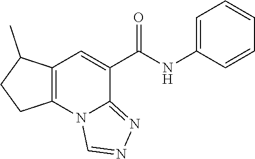 [SMILES: CC1CCc2c1cc(C(=O)Nc1ccccc1)c1nncn21]

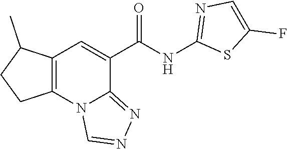 [SMILES: CC1CCc2c1cc(C(=O)Nc1ncc(F)s1)c1nncn21]

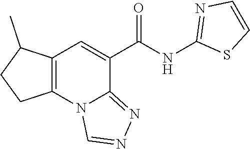 [SMILES: CC1CCc2c1cc(C(=O)Nc1nccs1)c1nncn21]

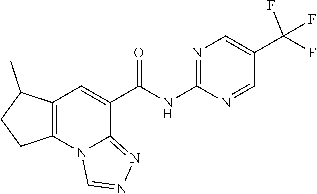 [SMILES: CC1CCc2c1cc(C(=O)Nc1ncc(C(F)(F)F)cn1)c1nncn21]

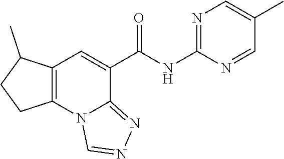 [SMILES: Cc1cnc(NC(=O)c2cc3c(n4cnnc24)CCC3C)nc1]

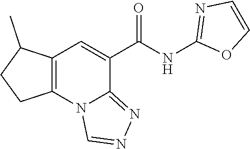 [SMILES: CC1CCc2c1cc(C(=O)Nc1ncco1)c1nncn21]

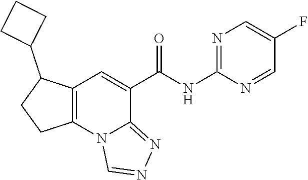 [SMILES: O=C(Nc1ncc(F)cn1)c1cc2c(n3cnnc13)CCC2C1CCC1]

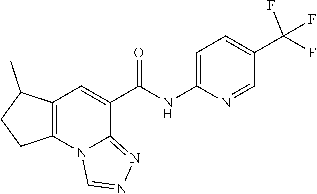 [SMILES: CC1CCc2c1cc(C(=O)Nc1ccc(C(F)(F)F)cn1)c1nncn21]

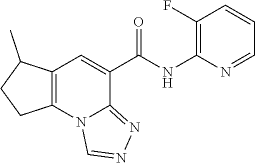 [SMILES: CC1CCc2c1cc(C(=O)Nc1ncccc1F)c1nncn21]

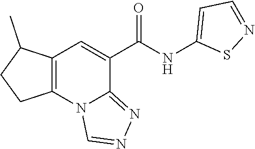 [SMILES: CC1CCc2c1cc(C(=O)Nc1ccns1)c1nncn21]

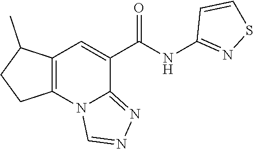 [SMILES: CC1CCc2c1cc(C(=O)Nc1ccsn1)c1nncn21]

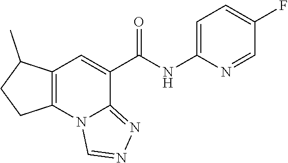 [SMILES: CC1CCc2c1cc(C(=O)Nc1ccc(F)cn1)c1nncn21]

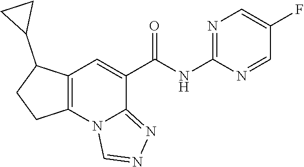 [SMILES: O=C(Nc1ncc(F)cn1)c1cc2c(n3cnnc13)CCC2C1CC1]

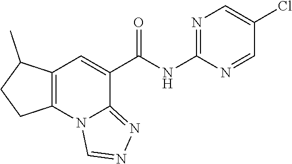 [SMILES: CC1CCc2c1cc(C(=O)Nc1ncc(Cl)cn1)c1nncn21]

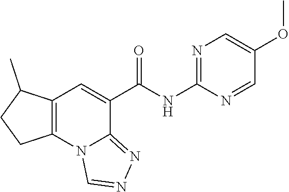 [SMILES: COc1cnc(NC(=O)c2cc3c(n4cnnc24)CCC3C)nc1]

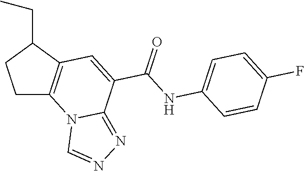 [SMILES: CCC1CCc2c1cc(C(=O)Nc1ccc(F)cc1)c1nncn21]

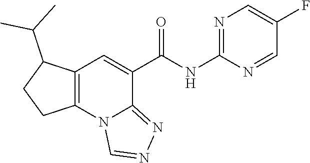 [SMILES: CC(C)C1CCc2c1cc(C(=O)Nc1ncc(F)cn1)c1nncn21]

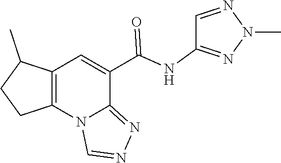 [SMILES: CC1CCc2c1cc(C(=O)Nc1cnn(C)n1)c1nncn21]

In some embodiments, provided here is a compound selected from the following table, a stereoisomer or mixture of stereoisomers thereof, or a pharmaceutically acceptable salt of each thereof:

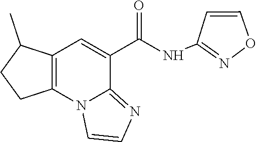 [SMILES: CC1CCc2c1cc(C(=O)Nc1ccon1)c1nccn21]

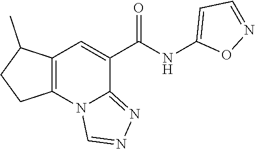 [SMILES: CC1CCc2c1cc(C(=O)Nc1ccno1)c1nncn21]

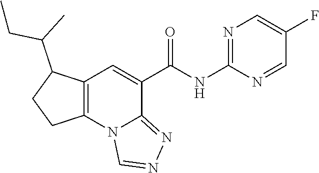 [SMILES: CCC(C)C1CCc2c1cc(C(=O)Nc1ncc(F)cn1)c1nncn21]

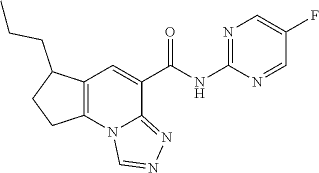 [SMILES: CCCC1CCc2c1cc(C(=O)Nc1ncc(F)cn1)c1nncn21]

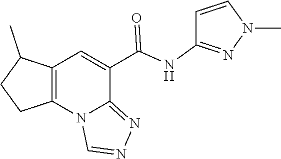 [SMILES: CC1CCc2c1cc(C(=O)Nc1ccn(C)n1)c1nncn21]

In some embodiments, provided here is a compound selected from:

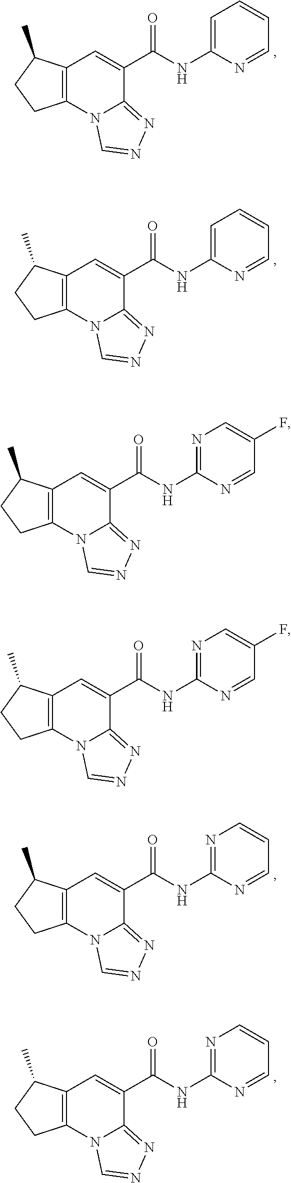 [SMILES: C[C@@H]1CCc2c1cc(C(=O)Nc1ccccn1)c1nncn21 ; C[C@H]1CCc2c1cc(C(=O)Nc1ccccn1)c1nncn21 ; C[C@@H]1CCc2c1cc(C(=O)Nc1ncc(F)cn1)c1nncn21 ; C[C@H]1CCc2c1cc(C(=O)Nc1ncc(F)cn1)c1nncn21 ; C[C@@H]1CCc2c1cc(C(=O)Nc1ncccn1)c1nncn21 ; C[C@H]1CCc2c1cc(C(=O)Nc1ncccn1)c1nncn21]

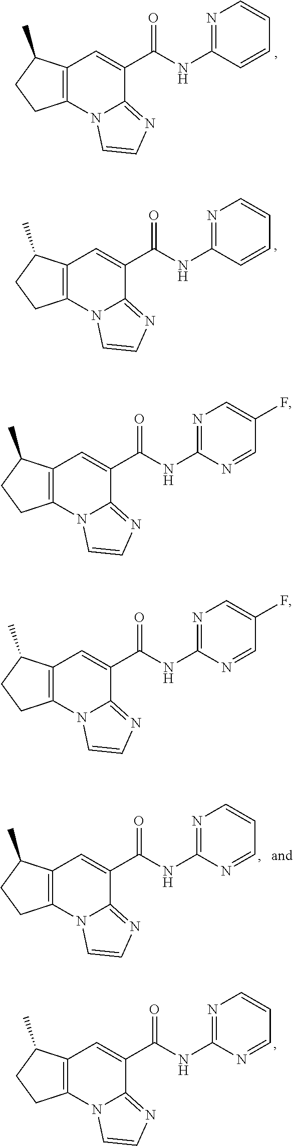 [SMILES: C[C@@H]1CCc2c1cc(C(=O)Nc1ccccn1)c1nccn21 ; C[C@H]1CCc2c1cc(C(=O)Nc1ccccn1)c1nccn21 ; C[C@@H]1CCc2c1cc(C(=O)Nc1ncc(F)cn1)c1nccn21 ; C[C@H]1CCc2c1cc(C(=O)Nc1ncc(F)cn1)c1nccn21 ; C[C@@H]1CCc2c1cc(C(=O)Nc1ncccn1)c1nccn21 ; C[C@H]1CCc2c1cc(C(=O)Nc1ncccn1)c1nccn21]

or a pharmaceutically acceptable salt of each thereof.

In some embodiments, provided here is a compound selected from the following table, a stereoisomer or mixture of stereoisomers thereof, or a pharmaceutically acceptable salt of each thereof:

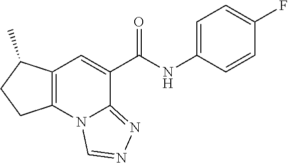 [SMILES: C[C@H]1CCc2c1cc(C(=O)Nc1ccc(F)cc1)c1nncn21]

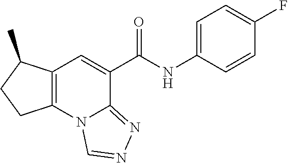 [SMILES: C[C@@H]1CCc2c1cc(C(=O)Nc1ccc(F)cc1)c1nncn21]

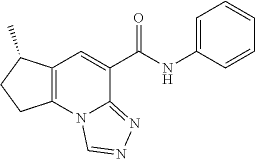 [SMILES: C[C@H]1CCc2c1cc(C(=O)Nc1ccccc1)c1nncn21]

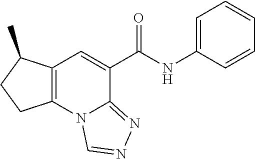 [SMILES: C[C@@H]1CCc2c1cc(C(=O)Nc1ccccc1)c1nncn21]

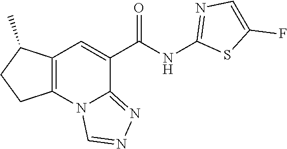 [SMILES: C[C@H]1CCc2c1cc(C(=O)Nc1ncc(F)s1)c1nncn21]

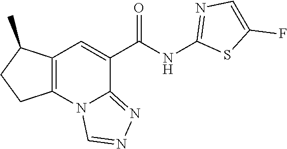 [SMILES: C[C@@H]1CCc2c1cc(C(=O)Nc1ncc(F)s1)c1nncn21]

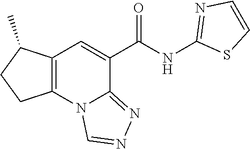 [SMILES: C[C@H]1CCc2c1cc(C(=O)Nc1nccs1)c1nncn21]

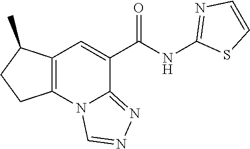 [SMILES: C[C@@H]1CCc2c1cc(C(=O)Nc1nccs1)c1nncn21]

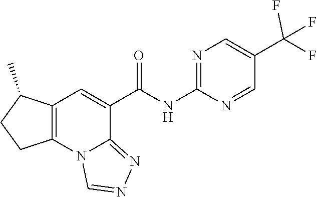 [SMILES: C[C@H]1CCc2c1cc(C(=O)Nc1ncc(C(F)(F)F)cn1)c1nncn21]

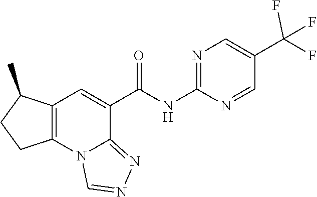 [SMILES: C[C@@H]1CCc2c1cc(C(=O)Nc1ncc(C(F)(F)F)cn1)c1nncn21]

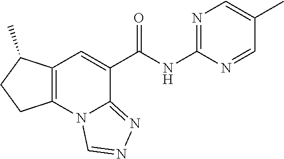 [SMILES: Cc1cnc(NC(=O)c2cc3c(n4cnnc24)CC[C@@H]3C)nc1]

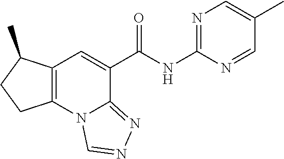 [SMILES: Cc1cnc(NC(=O)c2cc3c(n4cnnc24)CC[C@H]3C)nc1]

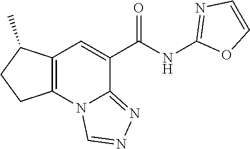 [SMILES: C[C@H]1CCc2c1cc(C(=O)Nc1ncco1)c1nncn21]

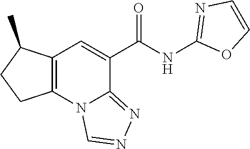 [SMILES: C[C@@H]1CCc2c1cc(C(=O)Nc1ncco1)c1nncn21]

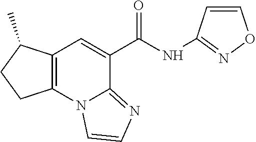 [SMILES: C[C@H]1CCc2c1cc(C(=O)Nc1ccon1)c1nccn21]

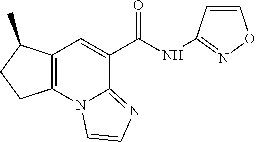 [SMILES: C[C@@H]1CCc2c1cc(C(=O)Nc1ccon1)c1nccn21]

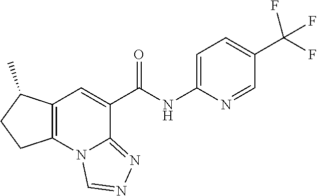 [SMILES: C[C@H]1CCc2c1cc(C(=O)Nc1ccc(C(F)(F)F)cn1)c1nncn21]

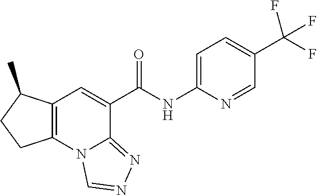 [SMILES: C[C@@H]1CCc2c1cc(C(=O)Nc1ccc(C(F)(F)F)cn1)c1nncn21]

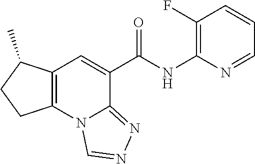 [SMILES: C[C@H]1CCc2c1cc(C(=O)Nc1ncccc1F)c1nncn21]

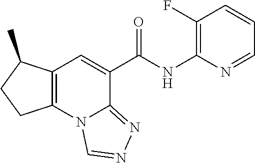 [SMILES: C[C@@H]1CCc2c1cc(C(=O)Nc1ncccc1F)c1nncn21]

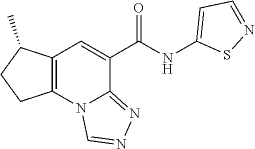 [SMILES: C[C@H]1CCc2c1cc(C(=O)Nc1ccns1)c1nncn21]

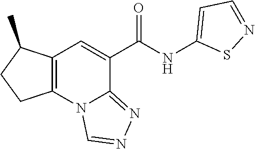 [SMILES: C[C@@H]1CCc2c1cc(C(=O)Nc1ccns1)c1nncn21]

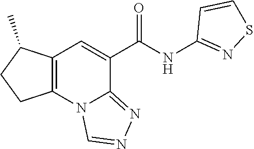 [SMILES: C[C@H]1CCc2c1cc(C(=O)Nc1ccsn1)c1nncn21]

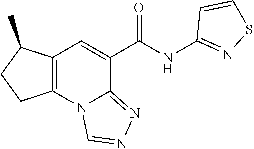 [SMILES: C[C@@H]1CCc2c1cc(C(=O)Nc1ccsn1)c1nncn21]

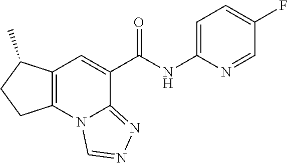 [SMILES: C[C@H]1CCc2c1cc(C(=O)Nc1ccc(F)cn1)c1nncn21]

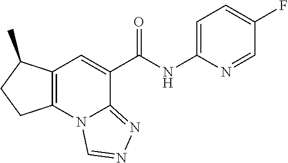 [SMILES: C[C@@H]1CCc2c1cc(C(=O)Nc1ccc(F)cn1)c1nncn21]

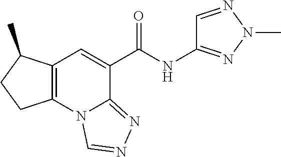 [SMILES: C[C@@H]1CCc2c1cc(C(=O)Nc1cnn(C)n1)c1nncn21]

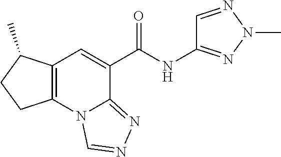 [SMILES: C[C@H]1CCc2c1cc(C(=O)Nc1cnn(C)n1)c1nncn21]

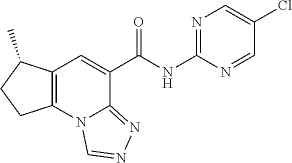 [SMILES: C[C@H]1CCc2c1cc(C(=O)Nc1ncc(Cl)cn1)c1nncn21]

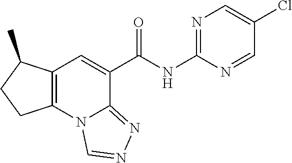 [SMILES: C[C@@H]1CCc2c1cc(C(=O)Nc1ncc(Cl)cn1)c1nncn21]

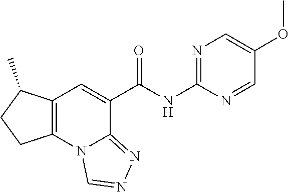 [SMILES: COc1cnc(NC(=O)c2cc3c(n4cnnc24)CC[C@@H]3C)nc1]

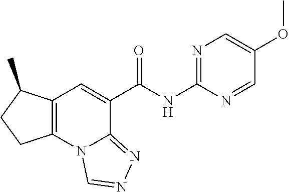 [SMILES: COc1cnc(NC(=O)c2cc3c(n4cnnc24)CC[C@H]3C)nc1]

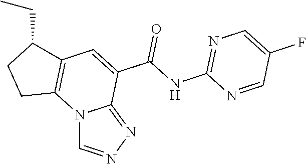 [SMILES: CC[C@H]1CCc2c1cc(C(=O)Nc1ncc(F)cn1)c1nncn21]

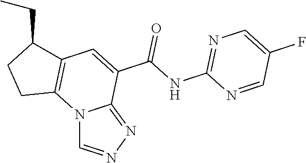 [SMILES: CC[C@@H]1CCc2c1cc(C(=O)Nc1ncc(F)cn1)c1nncn21]

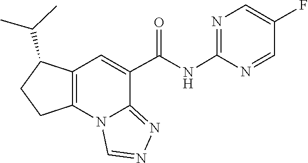 [SMILES: CC(C)[C@H]1CCc2c1cc(C(=O)Nc1ncc(F)cn1)c1nncn21]

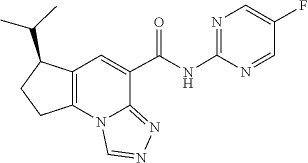 [SMILES: CC(C)[C@@H]1CCc2c1cc(C(=O)Nc1ncc(F)cn1)c1nncn21]

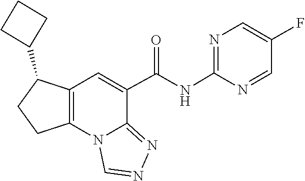 [SMILES: O=C(Nc1ncc(F)cn1)c1cc2c(n3cnnc13)CC[C@@H]2C1CCC1]

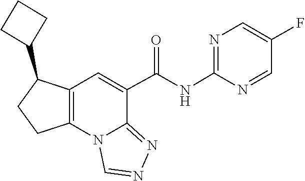 [SMILES: O=C(Nc1ncc(F)cn1)c1cc2c(n3cnnc13)CC[C@H]2C1CCC1]

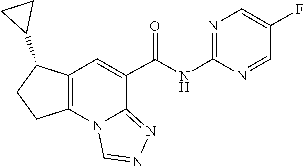 [SMILES: O=C(Nc1ncc(F)cn1)c1cc2c(n3cnnc13)CC[C@@H]2C1CC1]

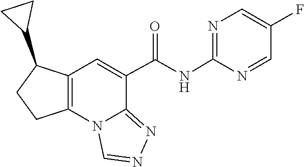 [SMILES: O=C(Nc1ncc(F)cn1)c1cc2c(n3cnnc13)CC[C@H]2C1CC1]

In some embodiments, provided here is a compound selected from:

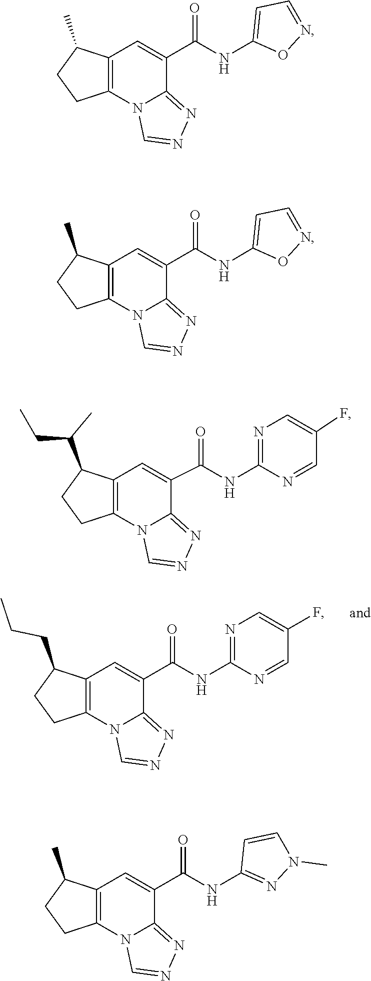 [SMILES: C[C@H]1CCc2c1cc(C(=O)Nc1ccno1)c1nncn21 ; C[C@@H]1CCc2c1cc(C(=O)Nc1ccno1)c1nncn21 ; CC[C@@H](C)[C@@H]1CCc2c1cc(C(=O)Nc1ncc(F)cn1)c1nncn21 ; CCC[C@@H]1CCc2c1cc(C(=O)Nc1ncc(F)cn1)c1nncn21 ; C[C@@H]1CCc2c1cc(C(=O)Nc1ccn(C)n1)c1nncn21]

or a pharmaceutically acceptable salt of each thereof:

In some embodiments, provided here is a compound of

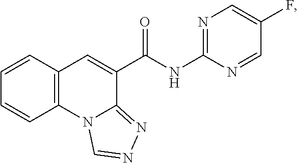 [SMILES: O=C(Nc1ncc(F)cn1)c1cc2ccccc2n2cnnc12]

or a pharmaceutically acceptable salt thereof.

In some embodiments, provided here is a compound of

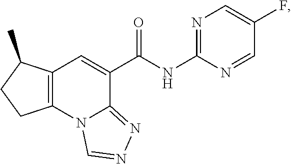 [SMILES: C[C@@H]1CCc2c1cc(C(=O)Nc1ncc(F)cn1)c1nncn21]

or a pharmaceutically acceptable salt of each thereof.

In some embodiments, provided here is a compound of

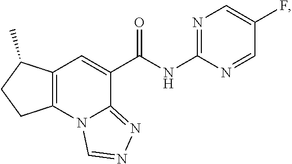 [SMILES: C[C@H]1CCc2c1cc(C(=O)Nc1ncc(F)cn1)c1nncn21]

or a pharmaceutically acceptable salt of each thereof.

In some embodiments, provided here is a compound of

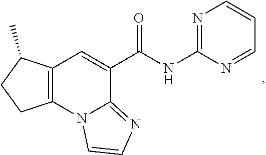 [SMILES: C[C@H]1CCc2c1cc(C(=O)Nc1ncccn1)c1nccn21]

or a pharmaceutically acceptable salt of each thereof.

The present disclosure further provides pharmaceutical composition, comprising a compound according to any of the above embodiments, a stereoisomer or mixture of stereoisomers thereof, or a pharmaceutically acceptable salt of each thereof, with one or more pharmaceutically acceptable carriers, diluents, or excipients.

The present disclosure provides a method of treating an immune-mediated disease in a patient, comprising administering to a patient in need of such treatment an effective amount of a compound, a stereoisomer or mixture of stereoisomers thereof, or a pharmaceutically acceptable salt of each thereof, or pharmaceutical composition according to any of the above embodiments.

The present disclosure provides a method of treating an auto-antibody driven autoimmune diseases in a patient, comprising administering to a patient in need of such treatment an effective amount of a compound, a stereoisomer or mixture of stereoisomers thereof, or a pharmaceutically acceptable salt of each thereof, or pharmaceutical composition according to any of the above embodiments.

The present disclosure also provides a method of treating a disease or disorder selected from psoriasis, atopic dermatitis, ulcerative colitis, Crohn's disease, graft-versus-host disease, rheumatoid arthritis, and multiple sclerosis in a patient, comprising administering to a patient in need of such treatment an effective amount of a compound, a stereoisomer or mixture of stereoisomers thereof, or a pharmaceutically acceptable salt of each thereof, or pharmaceutical composition according to any of the above embodiments.

The present disclosure also provides a method of treating a disease or disorder selected from systemic lupus erythematosus (SLE), rheumatoid arthritis, and myasthenia gravis (MG).

The present disclosure provides a compound according to any of the above embodiments, a stereoisomer or mixture of stereoisomers thereof, or a pharmaceutically acceptable salt of each thereof, for use in therapy.

The present disclosure provides a compound according to any of the above embodiments, a stereoisomer or mixture of stereoisomers thereof, or a pharmaceutically acceptable salt of each thereof, for use in treatment of an immune-mediated disease in a patient.

The present disclosure provides a compound according to any of the above embodiments, a stereoisomer or mixture of stereoisomers thereof, or a pharmaceutically acceptable salt of each thereof, for use in treatment of an auto-antibody driven autoimmune diseases in a patient.

The present disclosure also provides a compound according to any of the above embodiments, a stereoisomer or mixture of stereoisomers thereof, or a pharmaceutically acceptable salt of each thereof, for use in the treatment of a disease or disorder selected from psoriasis, atopic dermatitis, ulcerative colitis, Crohn's disease, graft-versus-host disease, rheumatoid arthritis, and multiple sclerosis.

The present disclosure also provides a compound according to any of the above embodiments, a stereoisomer or mixture of stereoisomers thereof, or a pharmaceutically acceptable salt of each thereof, for use in the treatment of a disease or disorder selected from systemic lupus erythematosus (SLE), rheumatoid arthritis, and myasthenia gravis (MG).

Furthermore, the present disclosure provides the use of a compound according to any of the above embodiments, a stereoisomer or mixture of stereoisomers thereof, or a pharmaceutically acceptable salt of each thereof, for the manufacture of a medicament for the treatment of an immune-mediated disease. In addition, the present disclosure provides the use of a compound according to any of the above embodiments, a stereoisomer or mixture of stereoisomers thereof, or a pharmaceutically acceptable salt of each thereof, for the manufacture of a medicament for the treatment of a disease or disorder selected from psoriasis, atopic dermatitis, ulcerative colitis, Crohn's disease, graft-versus-host disease, rheumatoid arthritis, and multiple sclerosis.

As used herein, the term “alkyl”, used alone or as part of a larger moiety, refers to a saturated, straight, or branched chain hydrocarbon group containing one or more carbon atoms.

As used herein, the term “aryl”, used alone or as part of a larger moiety, refers to an aromatic hydrocarbon group, having 6, 10, or 14 π-electrons shared in a cyclic array.

Aryl can be monocyclic (having one ring), bicyclic (having two rings), or polycyclic (having two or more rings). Exemplary aryl includes phenyl, naphthyl, anthracenyl, and phenanthrenyl.

As used herein, the term “chiral phosphine ligand” refers to a class of organophosphine compounds useful as ligands to metals to form metal complexes, where the chirality arises either from their carbon backbone or from the phosphorous. The term “P-chiral phosphine ligand” refers to a subset of “chiral phosphine ligand” where the chirality arises from a stereogenic phosphorous atom (P*). Examples of chiral phosphine ligands and P-chiral phosphine ligands are described in Imamoto, et. al., Proc. Jpn. Acad. Ser. B. Phys. Biol. Sci. 2021, Nov. 11; 97(9): 520-542.

As used herein, the term “ChenPhos” refers to a class of chiral phosphine ligands, such as those described in Chen, W., et al., Angew. Chem. Int. Ed., 52: 8652-8656. For example, ChenPhos may have a structure of formula:

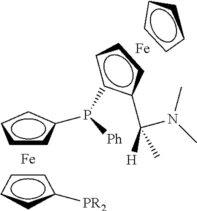

wherein R is aryl or alkyl. Examples of suitable R group include cyclohexyl, phenyl, t-butyl, isopropyl, ethyl, 4-fluorophenyl (4-FC₆H₄), 4-trifluromethylphenyl (4-CF₃C₆H₄), 2-norbornyl, 2-furyl, o-anisidyl, 3,5-dimethylphenyl (3,5-(CH₃)₂C₆H₃), 3,5-di-trifluoromethylphenyl (3,5-(CF₃)₂C₆H₃), 3,5-dimethyl-4-methoxy-phenyl (3,5-(CH₃)₂-4-(CH₃O)—C₆H₂), 1-naphthyl, among others. ChenPhos, along with others, is a subset of P-chiral phosphine ligands.

As used herein, the term “cycloalkyl” refers to a saturated ring system containing at least three carbon atoms. Cycloalkyl can be monocyclic (having one ring), bicyclic (having two rings), or polycyclic (having two or more rings). Exemplary monocyclic cycloalkyl rings include cyclopropyl, cyclobutyl, cyclopentyl, cyclohexyl, and cycloheptyl.

As used herein, the term “carbocycle” refers to a saturated or unsaturated ring system containing only carbons. Carbocycles include cycloalkyls and aryls and partially saturated rings.

As used herein, the term “halo” refers to halogen as a substituent, and specifically chloro, fluoro, bromo, or iodo.

As used herein, the term “heterocyclic” and “heterocycle” refers to an optionally substituted saturated ring system containing at least two carbon atoms and at least one heteroatom. Exemplary heteroatoms are oxygen, nitrogen, and sulfur. Exemplary heterocyclic rings (or heterocycles) include oxirane, aziridine, oxetane, oxolane, pyrrolidine, piperidine, and morpholine. Heterocycles can be monocycles (having one ring), bicycles (having two rings), or polycycles (having two or more rings) that may be, for example, fused with each other.

As used herein, the term “heteroaryl” refers to groups having 5 to 10 ring atoms, preferably 5, 6, 9, or 10 ring atoms, having 6, 10, or 14 π-electrons shared in a cyclic array, and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, and pyrazinyl. The term “bicyclic heteroaryl” includes groups in which a heteroaryl ring is fused to one or more aryl, or heteroaryl rings. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, and quinoxalinyl.

When there are two or more rings, the rings may be arranged separate from each other or connected with each other. When two rings are connected with each other, they may be connected in a “fuse” arrangement (or connection motif), a “spiro” arrangement, or a “bridge” arrangement. As used herein, the term “fuse” refers to an arrangement where the two rings are connected with each other, side-by-side, sharing two “bridgehead” atoms that are directly and immediately connected to each other. The “fuse” connection motif differs from “spiro” connection motif in that there is one and only one “bridgehead” atom in the “spiro” motif, and differs from “bridge” connection motif in that the two “bridgehead” atoms are not immediately connected to each other in the “bridge” motif. When a first ring is “fused with” a second ring, the “bridgehead” atoms are construed as belonging to both rings. Accordingly, if an embodiment provided here describes one such ring being a six-membered “carbocycle,” then the six ring atoms include two “bridgehead” atoms and four additional atoms. And all these six ring atoms are carbons in order to be a “carbocycle.” For example, the group

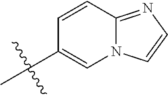 [SMILES: *c1ccc2nccn2c1]

falls outside the scope of “a 5-membered heteroaryl fused with a 6-membered carbocycle” because one bridgehead atom is not carbon.

As used herein, the term “oxo” refers to the oxygen atom as a substituent connected to another atom by a double bond. It may be denoted as “═O”. The term oxo is the carbonyl group less the carbon atom.

As used herein, the term “ortho-”, “meta-” and “para-” refers to the relative position between two substituents on a ring system. When two substituents are immediately adjacent to each other (i.e. directly bonded to two adjacent ring atoms), they are referred to as “ortho-” relative to each other. When they are separated by one other ring atom (in addition to the two ring atoms they are bonded to), they are referred to as “meta-” relative to each other. For a 6-membered ring system, when the two substituents are separated by two other ring atoms (in addition to the two ring atoms they are bonded to), they are referred to as “para-” relative to each other. For example, for the formula (A) below, A¹ and A², A² and A³, A³ and A⁴, A⁴ and A⁵, and A⁵ and A¹ are each considered ortho-relative to each other; A¹ and A³, A² and A⁴, A³ and A⁵, A⁴ and A¹, and A⁵ and A² of are each considered meta-relative to each other. For example, for the formula (B) below, A¹ and A², A² and A³, A³ and A⁴, A⁴ and A⁵, A⁵ and A⁶, and A⁶ and A¹ are each considered ortho-relative to each other; A¹ and A³, A² and A⁴, A³ and A⁵, A⁴ and A⁶, and A⁵ and A¹, and A⁶ and A² are each considered meta-relative to each other; A¹ and A⁴, A² and A⁵, and A³ and A⁶, are each considered para-relative to each other.

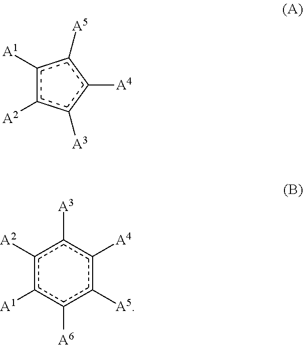

As used herein, [symbol] represents a bond that is part of an aromatic system. It may be properly and alternatively represented as a single bond or double bond depending on how the aromatic system is depicted. For example,

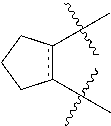

may be used to represent a 5-membered carbocycle fused with an aromatic system, such as a phenyl ring, at the two attachment points. The [symbol] bond may be properly represented as a single bond (e.g. as part of

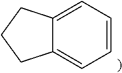

or as a double bond (e.g. as part of

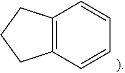

As used herein, the term “stereoisomer” refers to an isomer made up of the same atoms bonded by the same bonds but having different and non-interchangeable structures in the three-dimensional space. The term of stereoisomer includes “enantiomer” which refers to two stereoisomers that are mirror images of one another and are not superimposable over one another. A one-to-one mixture of a pair of enantiomers is referred to as a “racemic” mixture. The term of stereoisomer also includes “diastereoisomers” (or “diastereomer”) which refers to two stereoisomers that have at least two asymmetric atoms, but which are not mirror-images of each other. The absolute stereochemistry of a stereoisomer may be specified according to the Cahn-Ingold Prelog R S system, where the stereochemistry at each chiral center is designated as either R or S.

When stereoisomers are resolved yet whose absolute configuration is unknown, those stereocenters are designated (+) or (−) depending on the direction (dextro- or laevorotary) that they rotate the plane of polarization at the wavelength of the sodium D line. Unless explicitly stated otherwise, “enantiomer 1” refers to the enantiomer that eludes out first from the column during chiral separation of a racemic mixture under a stated separation condition; and “enantiomer 2” refers to the enantiomer that eludes out the second during the same separation. Occasionally, the order of elution differs between a preparatory column (e.g. for separation) and an analytical column (e.g. for purity assessment). For clarity, the designation of “enantiomer 1” and “enantiomer 2” is based on preparatory column. Moreover, there may be multiple stereocenters and two separations may be necessary to fully resolve the stereoisomers. For example, a first separation results in two bands, the first band eluting out including “enantiomer 1/1” and “enantiomer 1/2”, and the second band eluting out including “enantiomer 2/1” and “enantiomer 2/2”. A subsequent separation (e.g. using the same or different column conditions) may be used to resolve between “enantiomer 1/1” (the first eluting band in the subsequent separation) and “enantiomer 1/2” (the second eluting band in the subsequent separation).

As used herein, the term “immune-mediated disease” encompasses a group of autoimmune or inflammatory disorders in which immunological pathways play an important etiological and/or pathogenetic role. Such diseases are sometimes characterized by an alteration in cellular homeostasis. Immune-mediated diseases may be triggered by environmental factors, dietary habits, infectious agents, and genetic predisposition. Immune-mediated disease includes, for example, psoriasis, atopic dermatitis, ulcerative colitis, Crohn's disease, graft-versus-host disease, rheumatoid arthritis, and multiple sclerosis. Immune-mediated diseases may be mediated by auto-antibodies, T cells, cytokines, complement, or others.

As used herein, the term “auto-antibody driven autoimmune disease” refers to a type of autoimmune disease that occurs when the immune system produces antibodies that attack the body's own tissues or organs, causing inflammation and damage. Auto-antibody driven autoimmune diseases includes, for example, systemic lupus erythematosus (SLE), rheumatoid arthritis (RA), and myasthenia gravis (MG).

As used herein, the term “treating” includes restraining, slowing, stopping, or reversing the progression or severity of an existing symptom or disorder.

As used herein, the term “patient” refers to a human.

As used herein, the term “effective amount” refers to the amount or dose of compound of the disclosure, or a pharmaceutically acceptable salt thereof which, upon single or multiple dose administration to the patient, provides the desired effect in the patient under diagnosis or treatment.

An effective amount can be readily determined by one skilled in the art by the use of known techniques. In determining the effective amount for a patient, a number of factors are considered, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; the individual patient's medical history; and other relevant circumstances.

The compounds of the present disclosure are generally effective over a wide dosage range. For example, dosages per day normally fall within the range of about 0.1 to about 15 mg/kg of body weight. In some instances, dosage levels below the lower limit of the aforesaid range may be more than adequate, while in other cases still larger doses may be employed with acceptable side effects, and therefore the above dosage range is not intended to limit the scope of the disclosure in any way.

The compounds of the present disclosure are preferably formulated as pharmaceutical compositions administered by any route which makes the compound bioavailable, including oral and transdermal routes. Most preferably, such compositions are for oral administration. Such pharmaceutical compositions and processes for preparing same are well known in the art (See, e.g., Remington: The Science and Practice of Pharmacy, A. Adejare, Editor, 23^{rd} Edition, Elsevier Academic Press, 2020).

The compounds of the present disclosure, or pharmaceutically acceptable salts thereof, may be prepared according to the following Preparations and Examples by methods well known and appreciated in the art. Suitable reaction conditions for the steps of these Preparations and Examples are well known in the art and appropriate substitutions of solvents and co-reagents are within the skill of the art. Likewise, it will be appreciated by those skilled in the art that synthetic intermediates may be isolated and/or purified by various well-known techniques as needed or desired, and that frequently, it will be possible to use various intermediates directly in subsequent synthetic steps with little or no purification. As an illustration, compounds of the preparations and examples can be isolated, for example, by silica gel purification, isolated directly by filtration, or crystallization. Furthermore, the skilled artisan will appreciate that in some circumstances, the order in which moieties are introduced is not critical. The particular order of steps required to produce the compounds of the present disclosure is dependent upon the particular compound being synthesized, the starting compound, and the relative lability of the substituted moieties, and is well appreciated by the skilled chemist. All substituents, unless otherwise indicated, are as previously defined, and all reagents are well known and appreciated in the art.

Certain abbreviations are as follows: “BSA” refers to bovine serum albumin; “CMV” refers to cytomegalovirus; “DCM” refers to dichloromethane; “DMA” refers to dimethylacetamide; “DMEM” refers to Dulbecco's modified eagle medium; “DMF” refers to dimethylformamide; “DMF-DMA” refers to N,N-dimethylformamide dimethyl acetal; “DMSO” refers to dimethyl sulfoxide; “DPBS” refers to Dulbecco's phosphate-buffered saline; “EGFP” refers to enhanced green fluorescence protein; “EtOAc” refers to ethyl acetate; “FBS” refers to fetal bovine serum; “hr/hrs” refers to hour/hours; “MeOH” refers to methanol; “min” refers to minute/minutes; “SFC” refers to supercritical fluid chromatography; and “THF” refers to tetrahydrofuran.

In an optional step, a pharmaceutically acceptable salt of a compound according to any of the above embodiments can be formed by reaction of an appropriate free base of the compound with an appropriate pharmaceutically acceptable acid in a suitable solvent under standard conditions. The formation of such salts is well known and appreciated in the art. See, for example, Gould, P. L., “Salt selection for basic drugs,” International Journal of Pharmaceutics, 33: 201-217 (1986); Bastin, R. J., et al. “Salt Selection and Optimization Procedures for Pharmaceutical New Chemical Entities,” Organic Process Research and Development, 4: 427-435 (2000); and Berge, S. M., et al., “Pharmaceutical Salts,” Journal of Pharmaceutical Sciences, 66: 1-19, (1977). “Salt selection for basic drugs,” International Journal of Pharmaceutics, 33: 201-217 (1986). One of ordinary skill in the art will appreciate that a compound according to any of the above embodiments is readily converted to and may be isolated as a pharmaceutically acceptable salt.

The compounds of Formula I, Formula II, Formula III, Formula IV, a stereoisomer or mixture of stereoisomers thereof, or a pharmaceutically acceptable salt of each thereof, may be prepared by a variety of procedures known in the art, some of which are illustrated in the Schemes, Preparations, and Examples below. The specific synthetic steps for each of the routes described may be combined in different ways, or in conjunction with steps from different schemes, to prepare compounds of Formula I, Formula II, Formula III, Formula IV, a stereoisomer or mixture of stereoisomers thereof, or a pharmaceutically acceptable salt of each thereof. The products of each step in the scheme below can be recovered by conventional methods well known in the art, including, e.g. extraction, evaporation, precipitation, chromatography, filtration, trituration, and crystallization. In the scheme below, all variables and substituents unless otherwise indicated, are as previously defined. The reagents and starting materials are readily available to one of ordinary skill in the art.

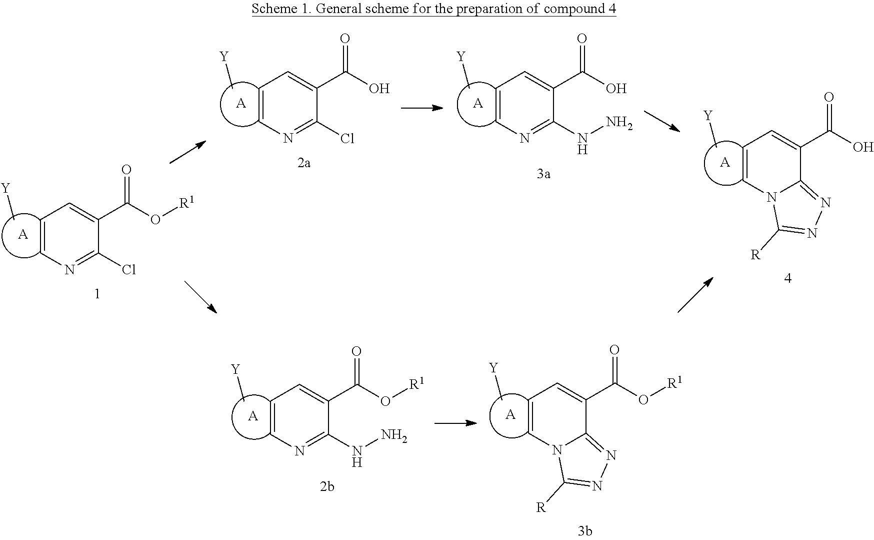

Scheme 1 depicts a general scheme for the synthesis of carboxylic acid compound 4. All variables are as defined above with respect to Formula I. Additionally, R¹ is an alkyl group, such as a C₁₋₃ alkyl. In some embodiments, R is hydrogen.

Compound 1 is hydrolyzed under conditions sufficient to provide carboxylic acid compound 2a. For example, compound 1 may be dissolved in a suitable organic solvent, water, or mixture thereof. An excess amount of base (e.g., lithium hydroxide) is then added to the solution and stirred at ambient temperature for several hours. At the completion of the reaction (e.g., by monitoring with Thin-Layer Chromatography (TLC)), the pH of the solution is adjusted to about 3 using an acid (e.g., 1N hydrochloric acid). The resulting mixture is then extracted with a suitable organic solvent (e.g., ethyl acetate), and the combined organic layers are dried (e.g., over anhydrous sodium sulfate), filtered, and concentrated under reduced pressure to provide compound 2a.

Compound 2a is then contacted with hydrazine under conditions sufficient (e.g., via nucleophilic aromatic substitution) to give compound 3a. For example, compound 2a is taken up in a suitable organic solvent (1,4-dioxane). An excess amount of hydrazine is then added to the solution and heated at a suitable temperature (e.g., 40-80° C.) for several hours. The reaction mixture is concentrated under reduced pressure to provide compound 3a.

Compound 3a is contacted with a suitable organic acid under conditions sufficient to provide compound 4. For example, a mixture of compound 3a and formic acid is heated at a suitable temperature (e.g., 60 to 100° C.) for several hours. The reaction mixture is concentrated under reduced pressure to provide compound 4, where R is hydrogen. In some embodiments, compound 4 may be taken to further reactions without workup or purification.

Alternatively, compound 1 is contacted with hydrazine under conditions sufficient provide compound 2b (e.g., via nucleophilic aromatic substitution). For example, compound 1 may be taken up in a suitable organic solvent (e.g., ethanol). An excess amount of hydrazine is then added to the solution and heated at a suitable temperature (e.g., 80-100° C.) for several hours. The reaction mixture is worked up (e.g., by quenching with a mild base), extracted with a suitable solvent (e.g., ethyl acetate), dried, concentrated under reduced pressure, and purified (e.g., silica gel chromatography) to give compound 2b.

Compound 2b is contacted with a suitable organic acid under conditions sufficient to provide compound 3b. For example, a mixture of compound 2b and formic acid can be heated at a suitable temperature (e.g., 60-100° C.) for several hours. The resulting mixture is then extracted with a suitable organic solvent (e.g., ethyl acetate), dried (e.g., anhydrous sodium sulfate), filtered, and concentrated under reduced pressure to provide compound 3b, where R is hydrogen.

Compound 3b is hydrolyzed under conditions sufficient to provide carboxylic acid compound 4. For example, compound 1 may be dissolved in a suitable organic solvent (e.g., THF, MeOH), water, or mixture thereof. An excess amount of base (e.g., lithium hydroxide) is then added to the solution and stirred at ambient temperature for several hours. At the completion of the reaction, the pH of the solution is adjusted to about 3 using an acid (e.g., 1N hydrochloric acid). The resulting mixture is then extracted with a suitable organic solvent (e.g., ethyl acetate), dried (e.g., anhydrous sodium sulfate), filtered, and concentrated under reduced pressure to provide compound 4.

In some embodiments, compound 1 of Scheme 1 is

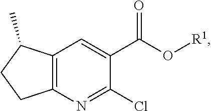

and compound 4 of Scheme 1 is

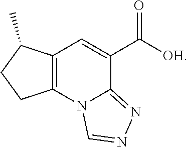 [SMILES: C[C@H]1CCc2c1cc(C(=O)O)c1nncn21]

In some embodiments,

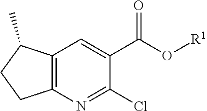

may be prepared by separating the racemic mixture of

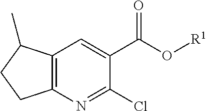

using a suitable method, such as via chiral separation with column chromatography under suitable conditions.

In some embodiments,

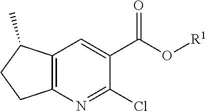

may be prepared from

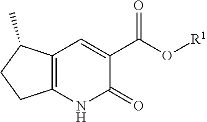

as provided in above embodiments (such as embodiment 58).

In some embodiments,

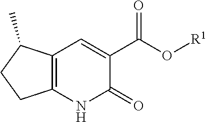

may be prepared by separating the racemic mixture of

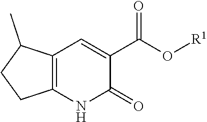

using a suitable method, such as via chiral separation with column chromatography under suitable conditions.

In some embodiments, provided herein is a method of preparing

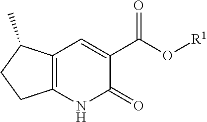

from

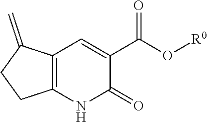

as described in embodiments above (such as embodiments 36 to 47).

In some embodiments, provided herein is a method of preparing

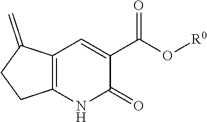

from

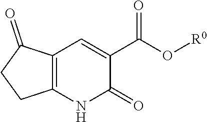

as described in embodiments above (such as embodiment 48).

In some embodiments, provided herein is a method of preparing

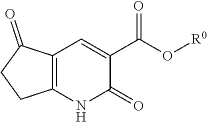

from

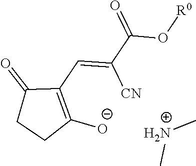

as described in embodiments above.

In some embodiments, provided herein is a method of preparing

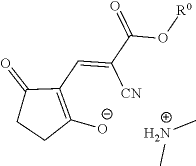

from

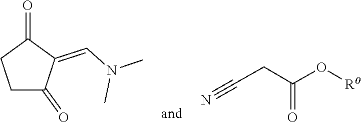

as described in embodiments above.

Alternatively, in some embodiments, provided herein is a method of preparing

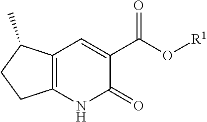

from

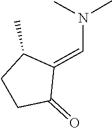

as described in embodiments above.

In some embodiments, provided herein is a method of preparing

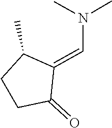

by contacting a racemic mixture of formula:
with a compound of formula:

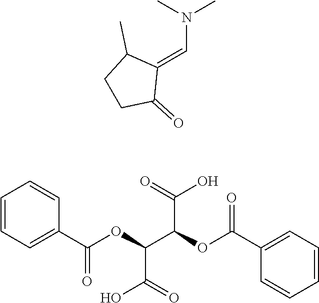 [SMILES: CC1CCC(=O)/C1=C/N(C)C ; O=C(O[C@H](C(=O)O)[C@H](OC(=O)c1ccccc1)C(=O)O)c1ccccc1]

at a condition sufficient to provide a co-crystal of formula:

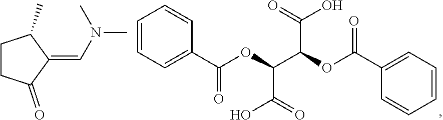

and contacting the co-crystal with an aqueous solution of sodium bicarbonate or potassium carbonate.

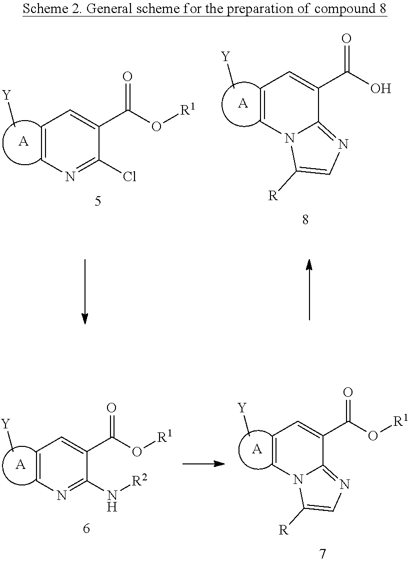

Scheme 2 depicts a general scheme for the synthesis of carboxylic acid intermediate 8. All variables are as defined above with respect to Formula I. Additionally, R² is H, —CH₂CH(OCH₃)₂, or —CH₂C(O)R. In some embodiments, R is hydrogen.

Compound 5 is converted under conditions sufficient to provide compound 6, such as by amination via a Buchwald reaction. For example, compound 5 is treated with tert-butyl carbamate, an appropriate palladium catalyst (e.g., palladium (II) acetate), an appropriate ligand (e.g., 2-dicyclohexylphosphio-2′,4′,6′-triisopropylbiphenyl), and an appropriate base (e.g., cesium carbonate) in a suitable solvent (e.g. toluene) heating at a suitable temperature (e.g. 60-120° C.) for several hours. The mixture is concentrated under reduced pressure and purified to give compound 6.

Alternatively, compound 5 is aminated with aminoacetaldehyde equivalent via nucleophilic aromatic substitution to compound 6. For example, compound 5 is treated with aminoacetaldehyde dimethyl acetal in a suitable solvent (e.g., ethanol) heating at a suitable temperature (e.g., 60-120° C.) for several hours. The mixture is concentrated under reduced pressure and purified to give compound 6.

Compound 6 is converted under conditions sufficient to provide imidazole compound 7, such as by cyclization with a haloacetaldehyde or a halomethylketone. For example, when R² is H, compound 6 is treated with chloroacetaldehyde in a suitable solvent (e.g., ethanol) and heated at a suitable temperature (e.g., 60-100° C.) for several hours. When R² is CH₂CH(OCH₃)₂, compound 6 is treated with halomethylketone in a suitable solvent (e.g., xylenes) and heated at a suitable temperature (e.g., 140° C.) for several hours. In either case, the mixture is concentrated under reduced pressure and purified to give compound 7.

Compound 7 is converted under conditions sufficient (e.g., by hydrolyzation) to give carboxylic acid intermediate compound 8. For example, compound 7 is taken up in a suitable organic solvent (e.g., THF, MeOH), water, or mixture thereof, treated with excess strong base (e.g., lithium hydroxide), and stirred at ambient temperature for several hours. The pH of the solution is adjusted to about 3 using an acid (e.g., 1N hydrochloric acid or similar). The solid was filtered and washed with water to give compound 8.

In some embodiments, compound 5 of Scheme 2 is

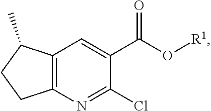

and compound 8 of Scheme 2 is

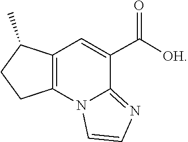 [SMILES: C[C@H]1CCc2c1cc(C(=O)O)c1nccn21]

Alternatively, when compound 1 or 5 is compound 11,

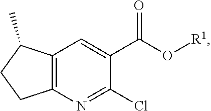

compound 4 and 8 can be prepared via Scheme 2a below, each designated as compound 14a and 14b, respectively:

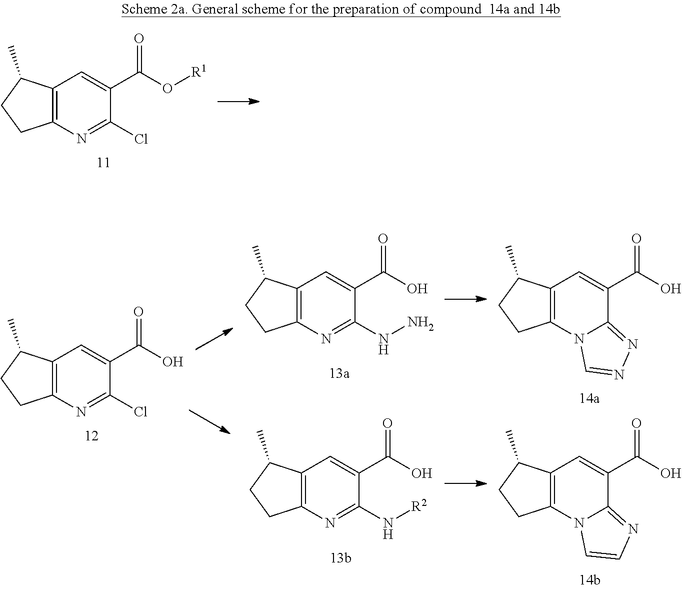

Scheme 2a depicts a general scheme for the synthesis of compounds 14a and 14b from compound 11. All variables are as defined above with respect to Schemes 1 and 2.

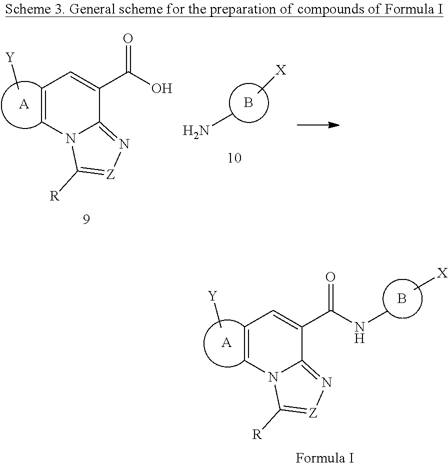

Scheme 3 depicts a general scheme for the synthesis of compounds of Formula I. All variables are as defined above with respect to Formula I. In some embodiments, the carboxylic acid compound 9 takes the form of carboxylic acid compound 14a or 14b.

Carboxylic acid compound 9 is contacted with an appropriate amine compound 10 under a condition sufficient to provide the compound of Formula I (e.g., via an amide coupling reaction). For example, carboxylic acid 9 and an appropriate amine compound 10 are taken up in a suitable solvent (e.g., dichloromethane), treated with an appropriate catalyst (e.g., pyridine) and an appropriate chlorinating agent (e.g., phosphoryl chloride), and stirred at ambient temperature for several hours. The reaction mixture is concentrated under reduced pressure, and the residue is triturated with a suitable solvent (e.g., DMF). The solid is filtered to give the compound of Formula I. In some embodiments, the intermediate compound 9 is the compound 4 described above. In some embodiments, the intermediate compound 9 is the compound 8 described above.

In some embodiments, the compound 10 is compound 10a, and the compound of Formula I is a compound of Formula II, wherein W is as defined above.

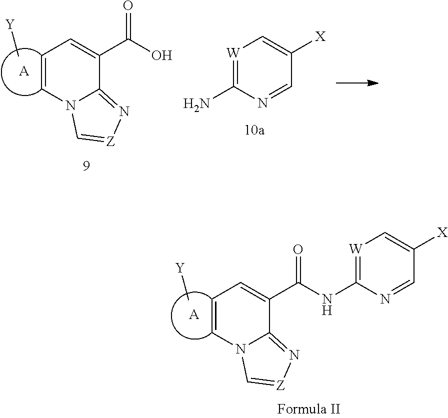

In some embodiments, the compound 10 is 10b, and the compound of Formula I is a compound of Formula II-1, wherein Q is as defined above.

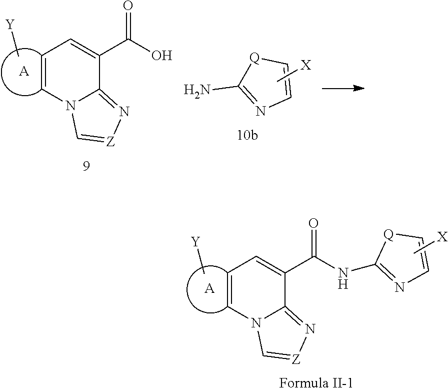

In some embodiments, the compound 10 is 10c, and the compound of Formula I is a compound of Formula II-2, wherein Q is as defined above.

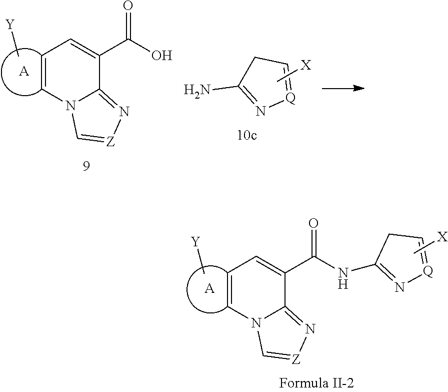

In some embodiments, the compound 10 is 10d, and the compound of Formula I is a compound of Formula II-3, wherein Q is as defined above.

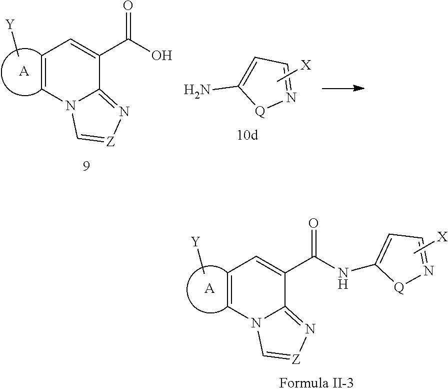

In some embodiments, the compound 10 is compound 10e, and the compound of Formula I is a compound of Formula II-4.

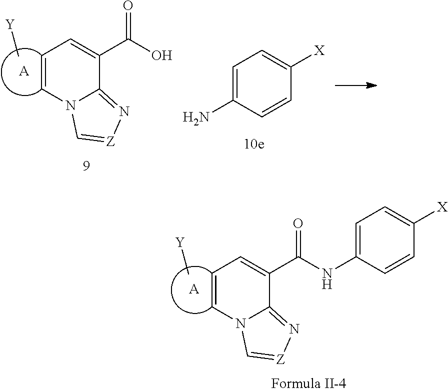

In some embodiments, ring A of compound 9 is phenyl, such that the compound of Formula II, when Y is hydrogen, also conforms to Formula IV.

In some embodiments, ring A of compound 9 is

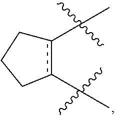

such that the compound of Formula II also includes a compound of Formula III.

In some embodiments, the compound of Formula I takes the form of Formula I-1, I-2, I-3, II-1, II-2, II-3, II-1A, III-1, III-2, III-3, III-1A, IV-1, IV-2, IV-3, IV-1A, V-1, V-2, V-3, or V-4, and can be prepared from suitable starting materials and methods similar to those described.

### Preparation 1

### [1,2,4]Triazolo[4,3-a]quinoline-4-carboxylic acid

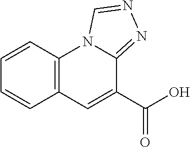 [SMILES: O=C(O)c1cc2ccccc2n2cnnc12]

### Step 1

### Ethyl 2-chloroquinoline-3-carboxylate

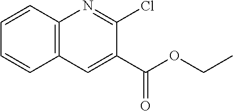 [SMILES: CCOC(=O)c1cc2ccccc2nc1Cl]

Ethyl-2-oxo-1,2-dihydroquinoline-3-carboxylate (1 g, 4.37 mmol) was mixed with N,N-diisopropylethylamine (2.29 mL, 13.1 mmol) and phosphoryl chloride (4.1 g, 26 mmol) in toluene (10 mL). The mixture was stirred at 120° C. for 18 hrs. After this time, the reaction mixture was concentrated under reduced pressure. The resulting residue was purified on a silica gel column (12 g) with ethyl acetate in petroleum ether as the eluant, at a concentration gradient of 0-10%, to give a clear, colorless oil. Trituration of this oil with petroleum ether (20 mL) at 20° C. for 10 min gave ethyl 2-chloroquinoline-3-carboxylate as a white solid (0.713 g, 61.5%). ES/MS (m/z): 236.1 (M+H).

### Step 2

### Ethyl 2-hydrazinylquinoline-3-carboxylate

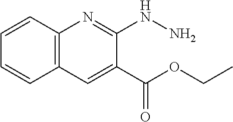 [SMILES: CCOC(=O)c1cc2ccccc2nc1NN]

Ethyl 2-chloroquinoline-3-carboxylate (500 mg, 1.89 mmol) was dissolved in ethanol (10 mL). Hydrazine (250 mg, 7.72 mmol) was introduced, and the mixture was stirred at 40° C. for 18 hrs. After this time, the reaction mixture was concentrated under reduced pressure. The resulting residue was purified on a silica gel column (20 g) with ethyl acetate in petroleum ether as the eluant, at a concentration gradient of 0-33%, to give ethyl 2-hydrazinylquinoline-3-carboxylate as a yellow solid (240 mg, 50.7%). ES/MS (m/z): 232.2 (M+H).

### Step 3

### Ethyl [1,2,4]triazolo[4,3-a]quinoline-4-carboxylate

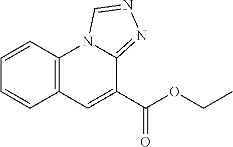 [SMILES: CCOC(=O)c1cc2ccccc2n2cnnc12]

Ethyl 2-hydrazinylquinoline-3-carboxylate (240 mg, 0.96 mmol) was stirred in formic acid (5 mL) at 100° C. for 18 hrs. After this time, the reaction was quenched with saturated aqueous sodium carbonate solution (30 mL) and extracted with ethyl acetate (30 mL×3). The organic layers were combined and dried over sodium sulfate, filtered, and concentrated under reduced pressure to give ethyl [1,2,4]triazolo[4,3-a]quinoline-4-carboxylate as a yellow solid (240 mg, 98.8%). ES/MS (m/z): 242.2 (M+H).

### Step 4

### [1,2,4]Triazolo[4,3-a]quinoline-4-carboxylic acid

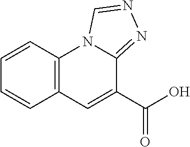 [SMILES: O=C(O)c1cc2ccccc2n2cnnc12]

Ethyl [1,2,4]triazolo[4,3-a]quinoline-4-carboxylate (240 mg, 0.945 mmol) was stirred in a mixture of THF (8 mL) and water (1 mL). Lithium hydroxide (200 mg, 4.77 mmol) was added, and the reaction mixture was stirred at ambient temperature for 18 hrs.

After this time, the reaction mixture was concentrated under reduced pressure to remove the volatile organics, and then the pH of the solution was adjusted to about 3 with the addition of 1N HCl aqueous solution. The solid was filtered and washed with water to give [1,2,4]triazolo[4,3-a]quinoline-4-carboxylic acid as a light yellow solid (204 mg, 96.5%). ES/MS (m/z): 214.2 (M+H).

### Preparation 2

### 6-Methyl-7,8-dihydro-6H-cyclopenta[e][1,2,4]triazolo[4,3-a]pyridine-4-carboxylic acid

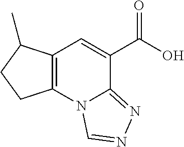 [SMILES: CC1CCc2c1cc(C(=O)O)c1nncn21]

### Step 1

### (Z)-2-((Dimethylamino)methylene)-3-methylcyclopentan-1-one

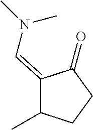

Cuprous iodide (56 g, 294 mmol) and tributylphosphine (150 mL, 570 mmol) were stirred in THF (300 mL) for 10 min under N₂. The mixture was then cooled to −78° C. After that, methyllithium (1.6 mol/L in diethyl ether) (180 mL, 290 mmol) was added dropwise. After the addition was complete, the mixture was further stirred at −78° C. for 30 min under N₂. Boron trifluoride diethyl etherate (34 mL, 268.8 mmol) was then added, and the mixture was stirred for another 5 min. Cyclopent-2-en-1-one (20 g, 243.6 mmol) was also added. This mixture was stirred at −68° C. for 10 min, warmed to −55° C. and stirred for 20 min, and then warmed again to −40° C. and stirred for 10 min under N₂. DMF-DMA (81 mL, 607 mmol) was then added, and the mixture was warmed to 20° C. and stirred for 16 hrs under N₂. The yellow mixture was poured into a saturated aqueous NaCl solution (500 mL) and extracted with EtOAc (4×200 mL). The organic layers were combined and dried over anhydrous sodium sulfate, filtered, and the solvent concentrated under reduced pressure. The residue was purified via flash silica gel chromatography (330 g) with EtOAc in petroleum ether as the eluant, at a concentration gradient of 0-100%, to give (Z)-2-((dimethylamino)methylene)-3-methylcyclopentan-1-one as a yellow oil (34 g, 82%). ES/MS (m/z): 154.2 (M+H).

### Step 2

### Methyl 5-methyl-2-oxo-2,5,6,7-tetrahydro-1H-cyclopenta[b]pyridine-3-carboxylate

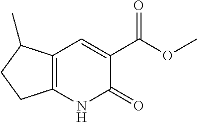

(Z)-2-((Dimethylamino)methylene)-3-methylcyclopentan-1-one (13.46 g, 87.85 mmol) was dissolved in MeOH (100 mL). To this mixture, piperidine (7.5 g, 88 mmol) was added followed by methyl cyanoacetate (17.6 g, 176 mmol). The mixture was heated to 80° C. under N₂ and maintained for 18 hrs. After this time, volatile organics were concentrated under reduced pressure, and the residue was purified on a silica gel column (120 g) with EtOAc in petroleum ether as the eluant, at a concentration gradient of 0-60%, to give methyl 5-methyl-2-oxo-2,5,6,7-tetrahydro-1H-cyclopenta[b]pyridine-3-carboxylate as a brown solid (14 g, 69%). ¹H NMR (CDCl₃): 7.96 (s, 1H), 7.21 (s, 1H), 3.86 (s, 3H), 3.12-3.03 (m, 1H), 2.92-2.82 (m, 2H), 2.35-2.28 (m, 1H), 1.64-1.55 (m, 1H), 1.19 (d, J=6.9 Hz, 3H).

### Step 3

### Methyl 2-chloro-5-methyl-6,7-dihydro-5H-cyclopenta[b]pyridine-3-carboxylate

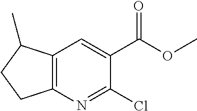

Methyl 5-methyl-2-oxo-2,5,6,7-tetrahydro-1H-cyclopenta[b]pyridine-3-carboxylate (2 g, 9.65 mmol) in toluene (5 mL) was added to N,N-diisopropylethylamine (3.3 mL, 19 mmol) followed by phosphoryl chloride (10 mL). The reaction mixture was heated to 100° C. for 16 hrs. After this time, the reaction mixture was concentrated under reduced pressure and purified via silica gel chromatography (40 g, 0-20% ethyl acetate in petroleum ether) to give methyl 2-chloro-5-methyl-6,7-dihydro-5H-cyclopenta[b]pyridine-3-carboxylate as a yellow oil (800 mg, 35.3%). ES/MS (m/z): 226.0 (M+H).

### Step 4

### 2-Chloro-5-methyl-6,7-dihydro-5H-cyclopenta[b]pyridine-3-carboxylic acid

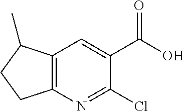

Methyl 2-chloro-5-methyl-6,7-dihydro-5H-cyclopenta[b]pyridine-3-carboxylate (800 mg, 3.4 mmol) was dissolved in a mixture of THF (6 mL), MeOH (3 mL), and water (1.5 mL). Lithium hydroxide (330 mg, 16.5 mmol) was added to this solution and stirred at 25° C. for 2 hrs. After this time, the pH of the solution was adjusted to about 3 using 1N HCl. This mixture was then extracted with ethyl acetate (10 mL×3). The combined organic layers were dried over sodium sulfate, filtered, and concentrated to dryness under reduced pressure. This gave 2-chloro-5-methyl-6,7-dihydro-5H-cyclopenta[b]pyridine-3-carboxylic acid as a white solid (570 mg, 79.1%). ES/MS (m/z): 212.1 (M+H).

### Step 5

### 2-Hydrazineyl-5-methyl-6,7-dihydro-5H-cyclopenta[h]pyridine-3-carboxylic acid

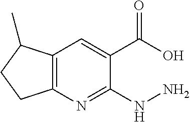

2-Chloro-5-methyl-6,7-dihydro-5H-cyclopenta[b]pyridine-3-carboxylic acid (400 mg, 1.89 mmol) was added to 1,4-dioxane (8 mL). To this mixture, hydrazine (2 g, 61.8 mmol) was added, and the mixture was stirred at 80° C. for 16 hrs. After this time, the reaction mixture was concentrated under reduced pressure to give 2-hydrazineyl-5-methyl-6,7-dihydro-5H-cyclopenta[b]pyridine-3-carboxylic acid as a yellow oil (400 mg, 91.9%). ES/MS (m/z): 208.2 (M+H).

### Step 6

6-Methyl-7,8-dihydro-6H-cyclopenta[e][1,2,4]triazolo[4,3-a]pyridine-4-carboxylic acid

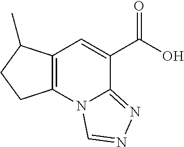 [SMILES: CC1CCc2c1cc(C(=O)O)c1nncn21]

2-Hydrazineyl-5-methyl-6,7-dihydro-5H-cyclopenta[b]pyridine-3-carboxylic acid (400 mg, 1.7 mmol) was added to formic acid (10 mL) and the mixture was stirred at 100° C. for 16 hrs. After this time, the mixture was concentrated to dryness under reduced pressure to give 6-methyl-7,8-dihydro-6H-cyclopenta[e][1,2,4]triazolo[4,3-a]pyridine-4-carboxylic acid as a yellow oil (320 mg, 59.4%). ES/MS (m/z): 218.2 (M+H).

### Preparation 3

### Imidazo[1,2-a]quinoline-4-carboxylic acid

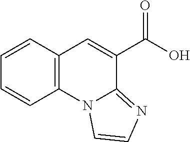 [SMILES: O=C(O)c1cc2ccccc2n2ccnc12]

### Step 1

### Ethyl 2-((2,2-dimethoxyethyll)amino)quinoline-3-carboxylate

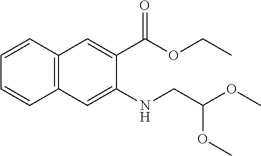 [SMILES: CCOC(=O)c1cc2ccccc2cc1NCC(OC)OC]

Ethyl 2-chloroquinoline-3-carboxylate (1 g, 4.12 mmol) was dissolved in ethanol (10 mL), and aminoacetaldehyde dimethyl acetal (4.6 mL, 42 mmol) was added to the solution. The mixture was heated to 80° C. and maintained for 2 days. After this time, this mixture was concentrated under reduced pressure, and the residue was purified via silica gel chromatography (12 g, 0-2% ethyl acetate in petroleum ether) to give ethyl 2-((2,2-dimethoxyethyl)amino)quinoline-3-carboxylate as a yellow oil (1.02 g, 78.3%). ES/MS (m/z): 305.3 (M+H).

### Step 2

### Ethyl imidazo[1,2-a]quinoline-4-carboxylate

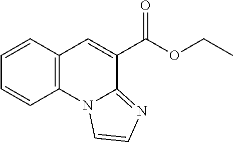 [SMILES: CCOC(=O)c1cc2ccccc2n2ccnc12]

Ethyl 2-((2,2-dimethoxyethyl)amino)quinoline-3-carboxylate (300 mg, 0.95 mmol) was stirred in xylenes (10 mL) with acetic acid (1 mL, 17.4 mmol) at 140° C. for 18 hrs. After this time, this mixture was concentrated under reduced pressure, and the residue was purified via silica gel chromatography (12 g, 0-50% ethyl acetate in petroleum ether) to give ethyl imidazo[1,2-a]quinoline-4-carboxylate as a grey solid (115 mg, 47.8%). ES/MS (m/z): 241.2 (M+H).

### Step 3

### Imidazo[1,2-a]quinoline-4-carboxylic acid

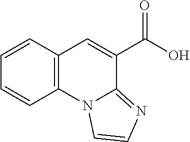 [SMILES: O=C(O)c1cc2ccccc2n2ccnc12]

Ethyl imidazo[1,2-a]quinoline-4-carboxylate (115 mg, 0.454 mmol) was stirred in a mixture of THF (2 mL) and water (0.5 mL). Lithium hydroxide (91 mg, 2.2 mmol) was added, and the mixture was stirred at ambient temperature for 18 hrs. After this time, the reaction mixture was concentrated under reduced pressure to remove the volatile organics. The pH of the solution was adjusted to about 3 with the addition of 1N HCl solution. This solid was filtered and washed with water to give imidazo[1,2-a]quinoline-4-carboxylic acid as a grey solid (60 mg, 60.3%). ES/MS (m/z): 213.1 (M+H).

### Preparation 4

### 6-Methyl-7,8-dihydro-6H-cyclopenta[e]imidazo[1,2-a]pyridine-4-carboxylic acid

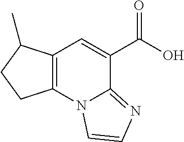 [SMILES: CC1CCc2c1cc(C(=O)O)c1nccn21]

### Step 1

### Methyl 2-amino-5-methyl-6,7-dihydro-5H-cyclopenta[b]pyridine-3-carboxylate

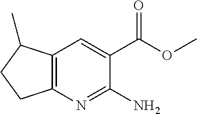

Methyl 2-chloro-5-methyl-6,7-dihydro-5H-cyclopenta[b]pyridine-3-carboxylate (2 g, 8.86 mmol) was added to toluene (20 mL). tert-Butyl carbamate (4.3 g, 36 mmol) was then added, followed by cesium carbonate (8.6 g, 26 mmol), palladium(II) acetate (400 mg, 1.8 mmol), and 2-dicyclohexylphosphio-2′,4′,6′-triisopropylbiphenyl (1.73 g, 3.6 mmol). This mixture was heated to 120° C. under nitrogen and maintained for 16 hrs. After this time, the mixture was concentrated under reduced pressure, and the residue was purified via silica gel chromatography (12 g, 0-40% ethyl acetate in petroleum ether) to give methyl 2-amino-5-methyl-6,7-dihydro-5H-cyclopenta[b]pyridine-3-carboxylate as a yellow oil (1.8 g, 89%). ES/MS (m/z): 207.1 (M+H).

### Step 2

### Methyl 6-methyl-7,8-dihydro-6H-cyclopenta[e]imidazo[1,2-a]pyridine-4-carboxylate

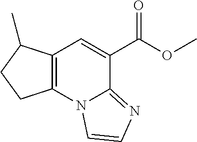 [SMILES: COC(=O)c1cc2c(n3ccnc13)CCC2C]

Methyl 2-amino-5-methyl-6,7-dihydro-5H-cyclopenta[b]pyridine-3-carboxylate (1.9 g, 8.3 mmol) was placed in ethanol (50 mL). Chloroacetaldehyde (3.3 g, 17 mmol) was added into the mixture, and stirred at 80° C. for 16 hrs. After this time, the mixture was concentrated under reduced pressure, and the residue was purified via silica gel chromatography (12 g, 0-40% ethyl acetate in petroleum ether) to give methyl 6-methyl-7,8-dihydro-6H-cyclopenta[e]imidazo[1,2-a]pyridine-4-carboxylate as a yellow solid (1.5 g, 63%). ES/MS (m/z): 231.1 (M+H).

### Step 3

### 6-Methyl-7,8-dihydro-6H-cyclopenta[e]imidazo[1,2-a]pyridine-4-carboxylic acid

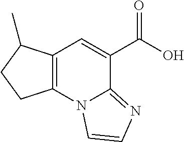 [SMILES: CC1CCc2c1cc(C(=O)O)c1nccn21]

Methyl 6-methyl-7,8-dihydro-6H-cyclopenta[e]imidazo[1,2-a]pyridine-4-carboxylate (1.5 g, 6.5 mmol) was stirred in a mixture of MeOH (6 mL), THE (12 mL), and water (3 mL). To this mixture, lithium hydroxide (640 mg, 26 mmol) was added and stirred at ambient temperature for 16 hrs. After this time, the reaction mixture was concentrated under reduced pressure to remove the volatile organics, then the pH of the solution was adjusted to about 3 with the addition of 1N HCl solution. The solid was filtered and washed with water to give 6-methyl-7,8-dihydro-6H-cyclopenta[e]imidazo[1,2-a]pyridine-4-carboxylic acid as an off-white solid (1 g, 71%). ES/MS (m/z): 216.9 (M+H).

### Preparation 5

### Methyl 2-chloro-5-cyclobutyl-6,7-dihydro-5H-cyclopenta[b]pyridine-3-carboxylate

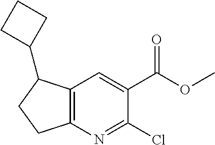 [SMILES: COC(=O)c1cc2c(nc1Cl)CCC2C1CCC1]

### Step 1

### Methyl 2-chloro-5-oxo-6,7-dihydro-5H-cyclopenta[b]pyridine-3-carboxylate

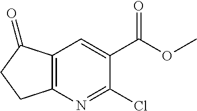

Phosphorus oxychloride (3 g, 0.02 mol) followed by diisopropylethylamine (2 g, 0.02 mol) was added dropwise to a solution of methyl 2,5-dioxo-2,5,6,7-tetrahydro-1H-cyclopenta[b]pyridine-3-carboxylate (2 g, 9 mmol) in toluene (20 mL). The reaction mixture was heated at 80° C. for 16 hrs. The reaction mixture was concentrated under reduced pressure to remove solvent. The residue was purified by flash silica gel chromatography eluting with a gradient of 0-40% EtOAc in hexane to give methyl 2-chloro-5-oxo-6,7-dihydro-5H-cyclopenta[b]pyridine-3-carboxylate (734 mg, 30%) as yellow solid. ES/MS (m/z): 225.9 (M+H).

### Step 2

### Methyl (Z)-2-chloro-5-(2-tosylhydrazineylidene)-6,7-dihydro-5H-cyclopenta[b]pyridine-3-carboxylate

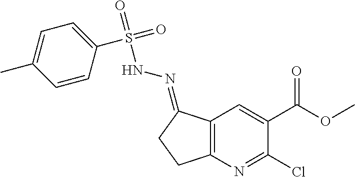

4-Methylbenzenesulfonic acid hydrazide (825 mg, 4.34 mmol) was added to a solution of methyl 2-chloro-5-oxo-6,7-dihydro-5H-cyclopenta[b]pyridine-3-carboxylate (1.00 g, 4.34 mmol) in MeOH (10.0 mL) at 25° C. The solution was stirred at ambient temperature under N₂ for 6 hrs. The resulting solid was filtered, washed with cold ether, and dried to give the methyl (Z)-2-chloro-5-(2-tosylhydrazineylidene)-6,7-dihydro-5H-cyclopenta[b]pyridine-3-carboxylate (1.75 g, 99+%) as white solid used without further purification. ES/MS (m/z): 393.9 (M+H).

### Step 3

### Methyl 2-chloro-5-cyclobutyl-6,7-dihydro-5H-cyclopenta[b]pyridine-3-carboxylate

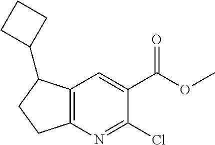 [SMILES: COC(=O)c1cc2c(nc1Cl)CCC2C1CCC1]

Cyclobutylboronic acid (673 mg, 6.67 mmol) was added to a solution of methyl (Z)-2-chloro-5-(2-tosylhydrazineylidene)-6,7-dihydro-5H-cyclopenta[b]pyridine-3-carboxylate (1.75 g, 4.44 mmol) and cesium carbonate (2.19 g, 6.67 mmol) in 1,4-dioxane (15.0 mL) under N₂. The mixture was heated at 110° C. for 16 hrs. The mixture was cooled to ambient temperature. Water (20 mL) was added to the mixture and the mixture extracted with EtOAc (50 mL×3). The combined organic extracts were washed with saturated aqueous NaCl and concentrated under reduced pressure to give the crude product. The crude product was purified by flash silica gel chromatography eluting with a gradient from 0-20% EtOAc in hexane to give methyl 2-chloro-5-cyclobutyl-6,7-dihydro-5H-cyclopenta[b]pyridine-3-carboxylate (451 mg, 37%) as yellow oil. ES/MS (m/z): 266.1 (M+H).

### Preparation 6

### Methyl (S)-5-methyl-2-oxo-2,5,6,7-tetrahydro-1H-cyclopenta[b]pyridine-3-carboxylate

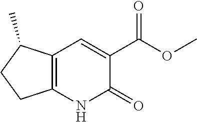

### Step 1

### (E)-2-(2-Cyano-3-methoxy-3-oxoprop-1-en-1-yl)-3-oxocyclopent-1-en-1-olate dimethylammonium

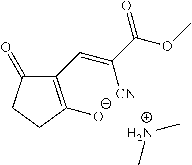

DMF-DMA (58.3 g, 489.3 mmol) was added to a solution of cyclopentane-1,3-dione (40 g, 407.7 mmol) in toluene (120 mL) at 0-10° C. The reaction mixture was stirred at 30-40° C. for 16 hrs. Toluene (160 mL) and methyl 2-cyanoacetate (48.5 g, 489.3 mmol) were added at 0-10° C. The reaction mixture was stirred at 25-35° C. for 16 hrs. After cooling, the reaction mixture was filtered and washed with toluene (160 mL). The cake was dried at 45-55° C. under reduced pressure to give (E)-2-(2-cyano-3-methoxy-3-oxoprop-1-en-1-yl)-3-oxocyclopent-1-en-1-olate dimethylammonium as brown-yellow solid (86.0 g, 78.2%). ES/MS m/z: 252.11 (C₁₂H₁₆N₂O₄); ES/MS m/z: 208.13 (M+H) as the freebase. ¹H NMR (DMSO-d6): δ ppm 2.10-2.30 (m, 4H), 2.55 (s, 6H), 3.65 (s, 3H), 7.62 (s, 1H), 7.97-8.38 (brs, 1H).

### Step 2

### Methyl 2,5-dioxo-2,5,6,7-tetrahydro-1H-cyclopenta[b]pyridine-3-carboxylate

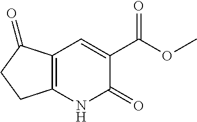

Acetic acid (228.5 g, 3.81 mol) was added to a solution of (E)-2-(2-cyano-3-methoxy-3-oxoprop-1-en-1-yl)-3-oxocyclopent-1-en-1-olate dimethylammonium (80 g, 317.1 mmol) in toluene (2.4 L) at 0-10° C. The reaction mixture was stirred at 85-95° C. for 16 hrs. After cooling, the reaction mixture was filtered and washed with toluene (160 mL). The cake was dried at 45-55° C. under reduced pressure to give methyl 2,5-dioxo-2,5,6,7-tetrahydro-1H-cyclopenta[b]pyridine-3-carboxylate as brown solid (39.1 g, 49.5%). ES/MS m/z: 208.06 (M+H). ¹H NMR (DMSO-d6): ppm 2.45-2.65 (m, 2H), 2.90-3.10 (m, 2H), 3.75 (s, 3H), 8.06 (s, 1H), 12.90 (brs, 1H).

### Step 3

### Methyl 5-methylene-2-oxo-2,5,6,7-tetrahydro-1H-cyclopenta[b]pyridine-3-carboxylate

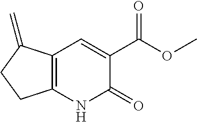

Methyl 2,5-dioxo-2,5,6,7-tetrahydro-1H-cyclopenta[b]pyridine-3-carboxylate (38.4 g, 185.2 mmol) was added to a solution of methyltriphenylphosphonium bromide (330.8 g, 926.0 mmol) and t-BuOK (99.7 g, 889 mmol) in toluene (1.15 L) at 20-30° C. The reaction mixture was stirred at 35-45° C. for 16 hrs. After cooling, the reaction mixture was filtered through diatomaceous earth (77 g, twice) and filtered. The filter cake was slurried with DCM (576 mL) and filtered. The filter cake was then again slurried with water/10% citric acid (pH: 6.2-6.8) and filtered. The filter cake was once again slurried with DCM (576 mL) and filtered. The filtrate was then concentrated under reduced pressure to provide methyl 5-methylene-2-oxo-2,5,6,7-tetrahydro-1H-cyclopenta[b]pyridine-3-carboxylate as a brown solid (24.0 g, 59.3%). ES/MS m/z: 206.08 (M+H). ¹H NMR (DMSO-d6): ppm 2.66-2.74 (m, 2H), 2.82-2.88 (m, 2H), 3.74 (s, 3H), 4.70-4.85 (m, 1H), 5.10-5.25 (m, 1H), 8.20 (s, 1H), 12.49 (brs, 1H).

### Step 4

### Methyl (S)-5-methyl-2-oxo-2,5,6,7-tetrahydro-1H-cyclopenta[b]pyridine-3-carboxylate

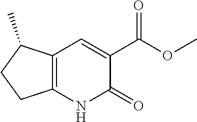

Bis(norbornadiene)rhodium(I) tetrafluoroborate (Rh(NBD)₂BF₄) (109 mg, 0.29 mmol) and 1-(dicyclohexylphosphino)-1′-[(S)-[(1R)-2-[(1R)-1-(dimethylamino)ethyl]ferrocenyl]phenylphosphino]ferrocene (CAS: 952586-19-5, 218 mg, 0.29 mmol) were added in MeOH (6.0 mL) under N₂ and stirred at 20-30° C. for 1 hr. MeOH (14.0 mL) and methyl 5-methylene-2-oxo-2,5,6,7-tetrahydro-1H-cyclopenta[b]pyridine-3-carboxylate (2.0 g, 9.62 mmol) were added, followed by flushing with hydrogen three times.

The reaction mixture was stirred at 25-35° C. under H₂ at 1.4-1.6 MPa for 16 h. The reaction mixture was concentrated and purified by column chromatography (DCM:MeOH, 200:1-100:1) to provide methyl (S)-5-methyl-2-oxo-2,5,6,7-tetrahydro-1H-cyclopenta[b]pyridine-3-carboxylate as a light yellow solid (1.3 g, 65.3%, 98.0% HPLC purity, 90.3% chiral purity). ES/MS m/z: 208.1 (M+H). ¹H NMR (DMSO-d6): ppm 1.15 (d, J=6.78 Hz, 3H), 1.49-1.61 (m, 1H), 2.19-2.32 (m, 1H), 2.70-2.80 (m, 2H), 2.97-3.05 (m, 1H), 3.71 (s, 3H) 7.95 (s, 1H) 12.23 (brs, 1H).

### Preparation 7

### Methyl (S)-5-methyl-2-oxo-2,5,6,7-tetrahydro-1H-cyclopenta[b]pyridine-3-carboxylate

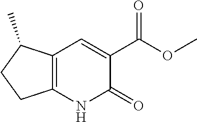

### Step 1

### (E)-2-((Dimethylamino)methylene)-3-methylcyclopentan-1-one

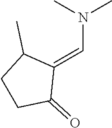

Tributyl phosphine (4.93 kg) was added dropwise to a solution of cuprous iodide (2.78 kg, 1.20 eq) in THE (20.0 L, 15 V) at 25° C. The mixture was stirred at 25° C. for 1 hr., and then cooled down to −70° C. Methyl lithium (2.5M, 5.85 L) was added dropwise to the cold mixture and stirred at −70° C. for 1 hr. Boron trifluoride diethyl etherate (1.90 kg) was added dropwise into the mixture and stirred for 1 hr. while maintaining the reaction temperature at −70° C. Then, cyclopent-2-en-1-one (1.00 kg) was added dropwise into the reaction mixture at −70° C. and stirred for 2 hrs., prior to the dropwise addition of DMF-DMA (2.90 kg) while maintaining the temperature at −70° C. After stirring at −70° C. for 1 hr., the reaction mixture was allowed to warm to 25° C. and maintained for 6 hrs. The reaction was quenched by slow addition over 30 min. of 15% sodium biphosphate (3V). The solids were filtered off, washed with EtOAc (1V). The mother liquors were concentrated under reduced pressure, extracted with EtOAc (2V×3) and concentrated under reduced pressure to give a crude mixture containing the desired product. The crude mixture was poured into acetonitrile (5V) and extracted with petroleum ether (5V×3). The phases were separated and concentrated separately. The acetonitrile phase gave 600 g (32%) of a residue containing desired product that was used in the next step without further purification.

### Step 2

### (S,E)-2-((Dimethylamino)methylene)-3-methylcyclopentan-1-one (2S,3S)-2,3-bis(benzoyloxy)succinate

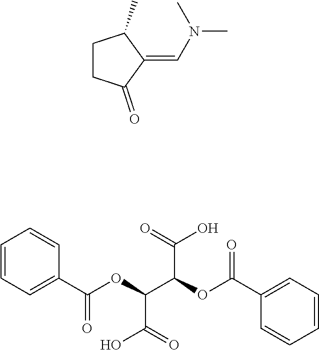 [SMILES: C[C@H]1CCC(=O)/C1=C/N(C)C ; O=C(O[C@H](C(=O)O)[C@H](OC(=O)c1ccccc1)C(=O)O)c1ccccc1]

A solution of (E)-2-((dimethylamino)methylene)-3-methylcyclopentan-1-one (1.60 kg, crude from step 1) in butanone (5V) at 25° C. was treated with a solution of (2S, 3S)-2,3-dibenzoyloxybutanedioic acid (0.6 equivalents) in butanone (15 V). The mixture was stirred at 60° C. for 2 hrs. Then, the reaction was cooled down to ambient temperature and stirred for 12 hrs. The mixture was filtered and washed with butanone to give a (S,E)-2-((dimethylamino)methylene)-3-methylcyclopentan-1-one (2S,3S)-2,3-bis(benzoyloxy)succinate crude salt with a chiral purity of 85%. The salt was treated with a mixture of acetonitrile/water (5V/1V), heated at 65° C. for 2 hrs., and then cooled at ambient temperature for 12 hrs. The solid was filtered, the cake was washed with acetonitrile (1V), and dried under vacuum to give (S,E)-2-((dimethylamino)methylene)-3-methylcyclopentan-1-one (2S,3S)-2,3-bis(benzoyloxy)succinate (6.4 kg, 23%, 99% optical purity) as a white solid.

### Step 3

### (S,E)-2-((Dimethylamino)methylene)-3-methylcyclopentan-1-one

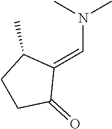

A solution of (S,E)-2-((dimethylamino)methylene)-3-methylcyclopentan-1-one (2S,3S)-2,3-bis(benzoyloxy)succinate (4.05 kg) in water (20.2L, 5V) and DCM (8.10L, 2V) was treated with sodium bicarbonate (1.12 kg, 2.00 eq) and the mixture stirred at 25° C. for 30 min. The organics were extracted with DCM (2V×3), washed with saturated aqueous NaCl, dried over anhydrous sodium sulfate, filtered, and concentrated under reduced pressure to give (S,E)-2-((dimethylamino)methylene)-3-methylcyclopentan-1-one (1.1 kg, 88%) as a light-yellow oil.

### Step 4

### Methyl (S)-5-methyl-2-oxo-2,5,6,7-tetrahydro-1H-cyclopenta[b]pyridine-3-carboxylate

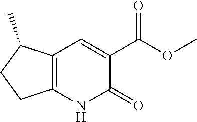

A solution of (S,E)-2-((dimethylamino)methylene)-3-methylcyclopentan-1-one (550 g) in MeOH (3.30 L, 6 V) was treated with methyl 2-cyanoacetate (582 g) and the mixture was stirred at 80° C. for 18 hrs. The reaction mixture was concentrated under reduced pressure to give a residue. The residue was then treated with EtOAc (2V), and the slurry was stirred at ambient temperature for 30 min. The solids were filtered, and the cake was washed with EtOAc (0.5V) and dried under vacuum to give methyl (S)-5-methyl-2-oxo-2,5,6,7-tetrahydro-1H-cyclopenta[b]pyridine-3-carboxylate (800 g, 55%) as a white solid.

### EXAMPLE 1

### N-(5-Fluoropyrimidin-2-yl)-[1,2,4]triazolo[4,3-a]quinoline-4-carboxamide

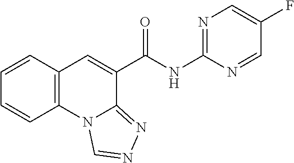 [SMILES: O=C(Nc1ncc(F)cn1)c1cc2ccccc2n2cnnc12]

[1,2,4]Triazolo[4,3-a]quinoline-4-carboxylic acid (100 mg, 0.447 mmol) and 5-fluoropyrimidine-2-amine (80 mg, 0.672 mmol) were dissolved in DCM (5 mL). To this mixture, pyridine (180 mg, 2.26 mmol) was added followed by phosphoryl chloride (350 mg, 2.24 mmol). The reaction mixture was stirred at ambient temperature for 18 hrs. After this time, the reaction mixture was concentrated under reduced pressure, and the residue was triturated with DMF at 20° C. for 5 min. The solid was filtered and washed with DMF to give N-(5-fluoropyrimidin-2-yl)-[1,2,4]triazolo[4,3-a]quinoline-4-carboxamide as a white solid (77 mg, 48.0%). ES/MS (m/z): 309.1 (M+H).

### EXAMPLE 2

### N-(5-Fluoropyrimidin-2-yl)imidazo[1,2-a]quinoline-4-carboxamide

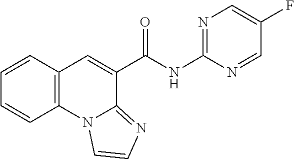 [SMILES: O=C(Nc1ncc(F)cn1)c1cc2ccccc2n2ccnc12]

Imidazo[1,2-a]quinoline-4-carboxylic acid was reacted with 5-fluoropyrimidine-2-amine using a procedure analogous to that of Example 1 to give N-(5-fluoropyrimidin-2-yl)imidazo[1,2-a]quinoline-4-carboxamide as white solid (166 mg, 95%). ES/MS (m/z): 308.3 (M+H).

### EXAMPLE 3

### (R)-6-Methyl-N-(pyridin-2-yl)-7,8-dihydro-6H-cyclopenta[e][1,2,4]triazolo[4,3-a]pyridine-4-carboxamide Or (S)-6-Methyl-N-(pyridin-2-yl)-7,8-dihydro-6H-cyclopenta[e][1,2,4]triazolo[4,3-a]pyridine-4-carboxamide (Enantiomer 1) and

### EXAMPLE 4

### (R)-6-Methyl-N-(pyridin-2-yl)-7,8-dihydro-6H-cyclopenta[e][1,2,4]triazolo[4,3-a]pyridine-4-carboxamide Or (S)-6-Methyl-N-(pyridin-2-yl)-7,8-dihydro-6H-cyclopenta[e][1,2,4]triazolo[4,3-a]pyridine-4-carboxamide (Enantiomer 2)

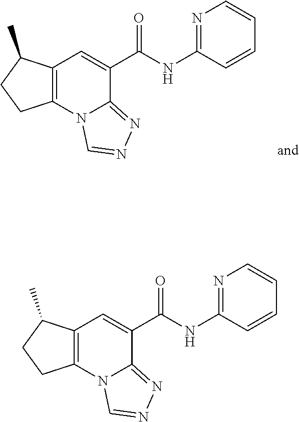 [SMILES: C[C@@H]1CCc2c1cc(C(=O)Nc1ccccn1)c1nncn21 ; C[C@H]1CCc2c1cc(C(=O)Nc1ccccn1)c1nncn21]

6-Methyl-7,8-dihydro-6H-cyclopenta[e][1,2,4]triazolo[4,3-a]pyridine-4-carboxylic acid (Preparation 2) (300 mg, 0.80 mmol) and pyridin-2-amine (120 mg, 1.6 mmol) were dissolved in dichloromethane (5 mL). To this mixture, pyridine (220 mg, 2.77 mmol) was added followed by phosphoryl chloride (200 mg, 1.3 mmol). The reaction mixture was stirred at ambient temperature for 18 hrs. After this time, the reaction mixture was poured into saturated aqueous NaCl (10 mL) and extracted with ethyl acetate (20 mL×3). The organic phases were combined and dried over sodium sulfate, filtered, and concentrated to dryness under reduced pressure. This residue was purified via silica gel chromatography (4 g, 0-80% ethyl acetate in petroleum ether) to provide a crude product. The crude product is subsequently subject to chiral separation on a preparatory column under the following conditions to give the enantiomers:

Separation Condition A:
Column: DAICEL CHIRALPAK AD 250×30 mm I.D., 10 μm
Mobile phase A: CO₂
Mobile phase B: Ethanol (0.1% NH40H)
Gradient: 40% B
Flow rate: 80 mL/Min

Analysis was done by Supercritical Fluid Chromatography (SFC) under the following condition to give the Retention Time (RT):
Column: Chiralpak OJ-3 50×4.6 mm I.D., 3 μm
Mobile phase A: CO₂
Mobile phase B: Ethanol (0.05% DEA)

Gradient: a) 0-2.5 min: 5% B—95% A ramping to 40% B—60% A;
b) 2.5 min-3.0 min: hold at 40% B—60% A;
c) 3.0 min-4.0 min: 40% B—60% A ramping to 5% B—95% A.

Flow rate: 4 ml/min

Column temperature: 35° C.

Automated backpressure regulator (ABPR) pressure: 1500 psi

Enantiomer 1 Retention time: 1.458 min

Enantiomer 2 Retention time: 1.566 min

Enantiomer 1 and enantiomer 2 are obtained at the above retention times (RT), and each dried to provide white solids. Enantiomer 1: (16.0 mg, 6.7%). ES/MS (m/z): 294.1 (M+H). Enantiomer 2: (17.6 mg, 7.4%). ES/MS (m/z): 294.1 (M+H).

The compounds in Table 1A were prepared using suitable starting material under conditions analogous to those of Examples 3 and 4. The starting material may be commercialized available or may be synthesized by referencing examples herein provided and common knowledge. The reaction time may be adjusted, for example, by monitoring with chromatography or similar means, to allow for reaction completion. Moreover, the separation and analytical conditions used are the same as those described for Examples 3 and 4.

TABLE 1A
Examples 5 to 8

| Ex. | | | ES/MS (m/z) | |
|---|---|---|---|---|
| No. | Chemical Name | Structure | (M + H) | RT (min) |
| 5 | (S)-N-(5- Fluoropyrimidin-2-yl)- 6-methyl-7,8-dihydro- 6H- cyclopenta[e][1,2,4]triazolo [4,3-a]pyridine-4- carboxamide | 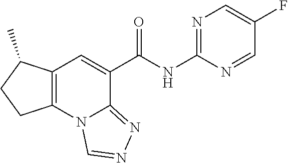 [SMILES: C[C@H]1CCc2c1cc(C(=O)Nc1ncc(F)cn1)c1nncn21] | 313.1 | 1.602 |
| 6 | (R)-N-(5- Fluoropyrimidin-2-yl)- 6-methyl-7,8-dihydro- 6H- cyclopenta[e][1,2,4]triazolo [4,3-a]pyridine-4- carboxamide | 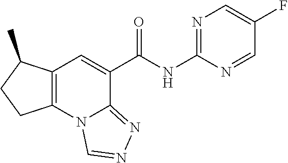 [SMILES: C[C@@H]1CCc2c1cc(C(=O)Nc1ncc(F)cn1)c1nncn21] | 313.1 | 1.769 |
| 7 | 6-Methyl-N-(pyrimidin- 2-yl)-7,8-dihydro-6H- cyclopenta[e][1,2,4] triazolo[4,3-a]pyridine-4- carboxamide (enantiomer 1) | 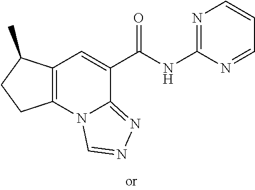 [SMILES: C[C@@H]1CCc2c1cc(C(=O)Nc1ncccn1)c1nncn21] | 295.1 | 1.629 |
| 8 | 6-Methyl-N-(pyrimidin- 2-yl)-7,8-dihydro-6H- cyclopenta[e][1,2,4]triazolo [4,3-a]pyridine-4- carboxamide (enantiomer 2) | 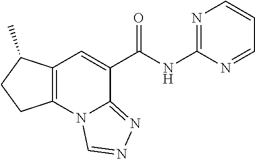 [SMILES: C[C@H]1CCc2c1cc(C(=O)Nc1ncccn1)c1nncn21] | 295.1 | 1.783 |

The compounds in Table 1B were prepared using suitable starting material under conditions analogous to those of Examples 3 and 4. The starting material may be commercialized available or may be synthesized by referencing examples herein provided and common knowledge. The reaction time may be adjusted, for example, by monitoring with chromatography or similar means, to allow for reaction completion. The crude product is subsequently subject to chiral separation on a preparatory column under Separation Condition A. Separation Condition A:

Column: DAICEL CHIRALPAK AD 250×30 mm I.D., 10 μm

Mobile phase A: CO₂

Mobile phase B: Ethanol (0.1% NH40H)

Gradient: 40% B

Flow rate: 80 mL/Min

Retention times are analyzed using an analytical column under the following condition, and the retention times are provided in Table 1B:

Column: (S,S)-Welk-0-1.8 50×4.6 mm I.D., 1.8 m

Mobile phase A: COS

Mobile phase B: 40 ethanol (0.05% DEA)

Flow rate: 2.8 mL/min

Column temperature: 35° C.

ABPR pressure: 1500 psi.

TABLE 1B
Examples 9 to 12

| | | | ES/MS | |
|---|---|---|---|---|
| Ex. | | | (m/z) | RT |
| No. | Chemical Name | Structure | (M + H) | (min) |
| 9 | 6-Methyl-N-(pyridin-2-yl)-7,8- dihydro-6H- cyclopenta[e]imidazo[1,2- a]pyridine-4-carboxamide (enantiomer 1) | 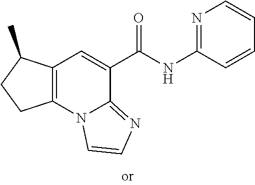 [SMILES: C[C@@H]1CCc2c1cc(C(=O)Nc1ccccn1)c1nccn21] | 293.0 | 1.614 |
| 10 | 6-Methyl-N-(pyridin-2-yl)-7,8- dihydro-6H- cyclopenta[e]imidazo[1,2- a]pyridine-4-carboxamide (enantiomer 2) | 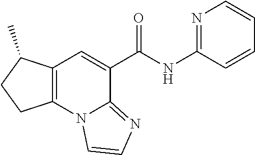 [SMILES: C[C@H]1CCc2c1cc(C(=O)Nc1ccccn1)c1nccn21] | 293.0 | 2.230 |
| 11 | 6-Methyl-N-(pyrimidin-2-yl)- 7,8-dihydro-6H- cyclopenta[e]imidazo[1,2- a]pyridine-4-carboxamide (enantiomer 1) | 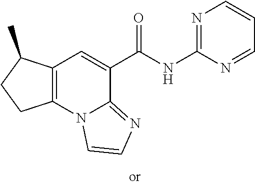 [SMILES: C[C@@H]1CCc2c1cc(C(=O)Nc1ncccn1)c1nccn21] | 294.1 | 3.231 |
| 12 | 6-Methyl-N-(pyrimidin-2-yl)- 7,8-dihydro-6H- cyclopenta[e]imidazo[1,2- a]pyridine-4-carboxamide (enantiomer 2) | 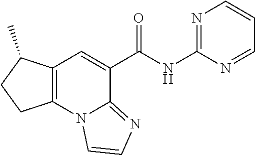 [SMILES: C[C@H]1CCc2c1cc(C(=O)Nc1ncccn1)c1nccn21] | 294.1 | 2.296 |

The compounds in Table 1C were prepared using suitable starting material under conditions analogous to those of Examples 3 and 4. The starting material may be commercialized available or may be synthesized by referencing examples herein provided and common knowledge. The reaction time may be adjusted, for example, by monitoring with chromatography or similar means, to allow for reaction completion. The crude product is subsequently subject to chiral separation on a preparatory column to give the enantiomers under conditions analogous to Separation Condition A (with necessary variation in the mobile phase (e.g. MeOH, EtOH, isopropanol, ACN or 1:1 mixtures thereof) used and gradient (e.g. 35% B, 40% B, 55% B, or 60% B) to optimize the performance). The analytical conditions used are as follows and the retention times are provided in Table 1C:

Column: AD-3 50×4.6 mm I.D., 3p m

Mobile phase A: CO₂

Mobile phase B: isopropanol (0.05% DEA)

Gradient: a) 0-2.5 min: 5% B—95% A ramping to 40% B—60% A;
b) 2.5 min-3.0 min: hold at 40% B—60% A;
c) 3.0 min-4.0 min: 40% B—60% A ramping to 5% B—95% A.

Flow rate: 4 ml/min

Column temperature: 35° C.

Automated backpressure regulator (ABPR) pressure: 1500 psi.

TABLE 1C
Examples 13 and 14

| Ex. | | | ES/MS (m/z) | |
|---|---|---|---|---|
| No. | Chemical Name | Structure | (M + H) | RT (min) |
| 13 | N-(5-Fluoropyrimidin- 2-yl)-6-methyl-7,8- dihydro-6H- cyclopenta[e]imidazo[1,2- a]pyridine-4- carboxamide (enantiomer 1) | 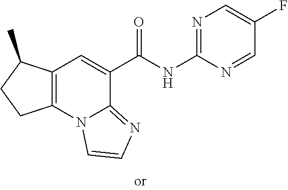 [SMILES: C[C@@H]1CCc2c1cc(C(=O)Nc1ncc(F)cn1)c1nccn21] | 312.1 | 2.282 |
| 14 | N-(5-Fluoropyrimidin- 2-yl)-6-methyl-7,8- dihydro-6H- cyclopenta[e]imidazo[1,2- a]pyridine-4- carboxamide (enantiomer 2) | 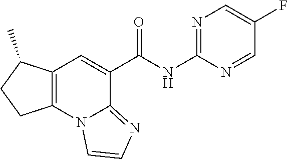 [SMILES: C[C@H]1CCc2c1cc(C(=O)Nc1ncc(F)cn1)c1nccn21] | 312.1 | 2.445 |

For clarity, enantiomer 1 above refers to the enantiomer that eludes out first from the stationary phase of the preparatory column; and enantiomer 2 above refers to the enantiomer that eludes out second from the stationary phase of the preparatory column.

Each compound described in Examples 1-14 are referred to as Compounds 1-14, respectively.

Compound 15 of Example 15 in Table 2 below can be synthesized according to the Schemes 1 and 3 described above with reference to conditions described for Examples 3 and 4 and separated using conditions similar to those described for Examples 3-14.

TABLE 2
Example 15

| Ex. No. | Chemical Name | Structure |
|---|---|---|
| 15 | N-(5-Fluoropyrimidin-2-yl)-7,8- dihydro-6H- cyclopenta[e][1,2,4]triazolo[4,3- a]pyridine-4-carboxamide | 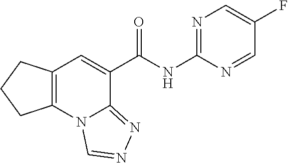 [SMILES: O=C(Nc1ncc(F)cn1)c1cc2c(n3cnnc13)CCC2] |

The compounds 16 to 57 of Table 2A below were synthesized according to the Schemes 1-3 described above with reference to conditions described for Examples 3 and 4 and separated on a preparatory column under conditions analogous to Separation Condition A (with necessary variation in the type of column, mobile phase and gradient as specified in Table 2A).

TABLE 2A
Examples 16 to 57

| | | | | Variation |
|---|---|---|---|---|
| | | | ES/MS | from |
| Ex. | | | (m/z) | Separation |
| No | Chemical Name | Structure | (M + H) | Condition A |
| 16 17 | N-(4-Fluorophenyl)-6- methyl-7,8-dihydro- 6H- cyclopenta[e][1,2,4] triazolo[4,3-a]pyridine-4- carboxamide (enantiomer 1) N-(4-Fluorophenyl)-6- methyl-7,8-dihydro- 6H- cyclopenta[e][1,2,4] triazolo[4,3-a]pyridine-4- carboxamide (enantiomer 2) | 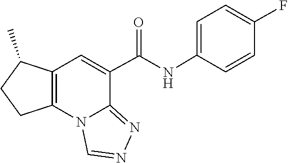 [SMILES: C[C@H]1CCc2c1cc(C(=O)Nc1ccc(F)cc1)c1nncn21] or 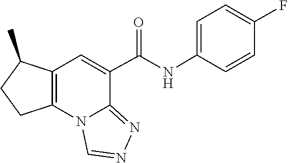 [SMILES: C[C@@H]1CCc2c1cc(C(=O)Nc1ccc(F)cc1)c1nncn21] | 310.9 311.0 | Column: DAICEL CHIRALCEL OX (250 mm * 30 mm, 10 μm) Gradient: Isocratic 40% B |
| 18 19 | 6-Methyl-N-phenyl- 7,8-dihydro-6H- cyclopenta[e][1,2,4] triazolo[4,3-a]pyridine-4- carboxamide (enantiomer 2) 6-Methyl-N-phenyl- 7,8-dihydro-6H- cyclopenta[e][1,2,4] triazolo[4,3-a]pyridine-4- carboxamide (enantiomer 1) | 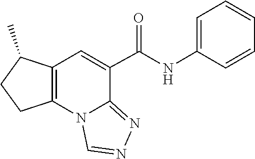 [SMILES: C[C@H]1CCc2c1cc(C(=O)Nc1ccccc1)c1nncn21] or 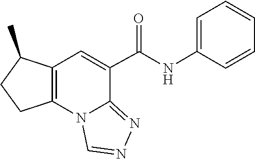 [SMILES: C[C@@H]1CCc2c1cc(C(=O)Nc1ccccc1)c1nncn21] | 292.9 293.0 | Column: DAICEL CHIRALPAK IC (250 mm * 30 mm, 10 μm) Mobile phase B: 1:1 ACN/Methanol (0.1% NH₄OH) Gradient: Isocratic 60% B |
| 20 21 | N-(2-methoxyphenyl)- 6-methyl-7,8-dihydro- 6H- cyclopenta[e][1,2,4] triazolo[4,3-a]pyridine-4- carboxamide (enantiomer 2) N-(2-methoxyphenyl)- 6-methyl-7,8-dihydro- 6H- cyclopenta[e][1,2,4] triazolo[4,3-a]pyridine-4- carboxamide (enantiomer 1) | 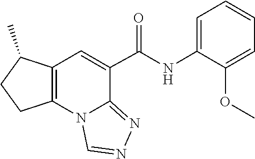 [SMILES: COc1ccccc1NC(=O)c1cc2c(n3cnnc13)CC[C@@H]2C] or 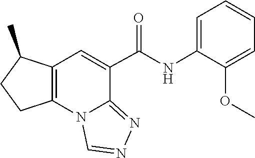 [SMILES: COc1ccccc1NC(=O)c1cc2c(n3cnnc13)CC[C@H]2C] | 323.2 323.2 | Column: DAICEL CHIRALCEL OX (250 mm * 30 mm, 10 μm) Mobile phase B: 1:1 ACN/Methanol (0.1% NH₄OH) Gradient: Isocratic 60% B |
| 22 23 | N-(5-Fluorothiazol-2- yl)-6-methyl-7,8- dihydro-6H- cyclopenta[e][1,2,4] triazolo[4,3-a]pyridine-4- carboxamide (enantiomer 1) N-(5-Fluorothiazol-2- yl)-6-methyl-7,8- dihydro-6H- cyclopenta[e][1,2,4] triazolo[4,3-a]pyridine-4- carboxamide (enantiomer 2) | 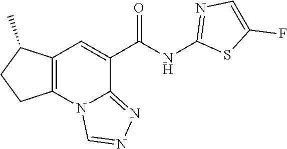 [SMILES: C[C@H]1CCc2c1cc(C(=O)Nc1ncc(F)s1)c1nncn21] or 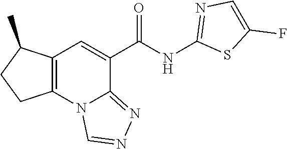 [SMILES: C[C@@H]1CCc2c1cc(C(=O)Nc1ncc(F)s1)c1nncn21] | 318.0 318.1 | Gradient: Isocratic 50% B Flow Rate: 120 mL/min |
| 24 25 | 6-Methyl-N-(thiazol-2- yl)-7,8-dihydro-6H- cyclopenta[e][1,2,4] triazolo[4,3-a]pyridine-4- carboxamide (enantiomer 2) 6-Methyl-N-(thiazol-2- yl)-7,8-dihydro-6H- cyclopenta[e][1,2,4] triazolo[4,3-a]pyridine-4- carboxamide (enantiomer 1) | 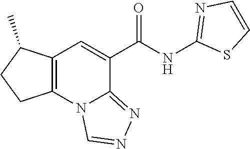 [SMILES: C[C@H]1CCc2c1cc(C(=O)Nc1nccs1)c1nncn21] or 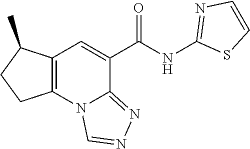 [SMILES: C[C@@H]1CCc2c1cc(C(=O)Nc1nccs1)c1nncn21] | 300.1 300.1 | Column: DAICEL CHIRALCEL OX (250 mm * 30 mm, 10 μm) Mobile phase B: Methanol (0.1% NH₄OH) Gradient: Isocratic 45% B |
| 26 27 | 6-Methyl-N-(5- (trifluoromethyl)pyrimidin- 2-yl)-7,8-dihydro- 6H- cyclopenta[e][1,2,4] triazolo[4,3-a]pyridine-4- carboxamide (enantiomer 1) 6-Methyl-N-(5- (trifluoromethyl)pyrimidin- 2-yl)-7,8-dihydro- 6H- cyclopenta[e][1,2,4] triazolo[4,3-a]pyridine-4- carboxamide (enantiomer 2) | 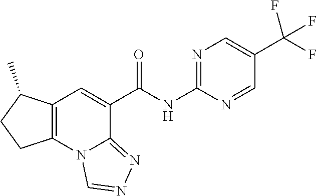 [SMILES: C[C@H]1CCc2c1cc(C(=O)Nc1ncc(C(F)(F)F)cn1)c1nncn21] or 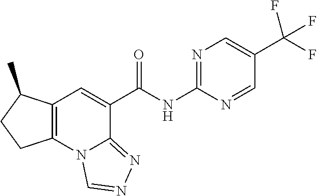 [SMILES: C[C@@H]1CCc2c1cc(C(=O)Nc1ncc(C(F)(F)F)cn1)c1nncn21] | 363.1 363.1 | Column: DAICEL CHIRALCEL OX (250 mm * 30 mm, 10 μm) Mobile phase B: Methanol (0.1% NH₄OH) Gradient: Isocratic 55% B |
| 28 29 | 6-Methyl-N-(5- methylpyrimidin-2-yl)- 7,8-dihydro-6H- cyclopenta[e][1,2,4]tria zolo[4,3-a]pyridine-4- carboxamide (enantiomer 2) 6-Methyl-N-(5- methylpyrimidin-2-yl)- 7,8-dihydro-6H- cyclopenta[e][1,2,4]tria zolo[4,3-a]pyridine-4- carboxamide (enantiomer 1) | 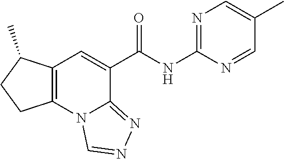 [SMILES: Cc1cnc(NC(=O)c2cc3c(n4cnnc24)CC[C@@H]3C)nc1] or 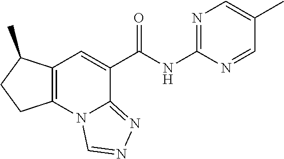 [SMILES: Cc1cnc(NC(=O)c2cc3c(n4cnnc24)CC[C@H]3C)nc1] | 309.2 309.4 | Column: DAICEL CHIRALCEL OX (250 mm * 30 mm, 10 μm) Mobile phase B: 1:1 ACN/Methanol (0.1% NH₄OH) Gradient: Isocratic 60% B |
| 30 31 | 6-Methyl-N-(oxazol-2- yl)-7,8-dihydro-6H- cyclopenta[e][1,2,4] triazolo[4,3-a]pyridine-4- carboxamide (enantiomer 1) 6-Methyl-N-(oxazol-2- yl)-7,8-dihydro-6H- cyclopenta[e][1,2,4] triazolo[4,3-a]pyridine-4- carboxamide (enantiomer 2) | 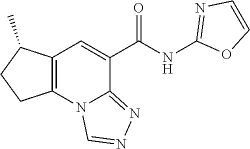 [SMILES: C[C@H]1CCc2c1cc(C(=O)Nc1ncco1)c1nncn21] or 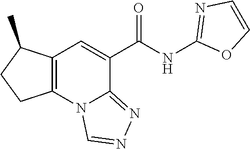 [SMILES: C[C@@H]1CCc2c1cc(C(=O)Nc1ncco1)c1nncn21] | 284.1 284.1 | Column: DAICEL CHIRALCEL OX (250 mm * 30 mm, 10 μm) Mobile phase B: 1:1 ACN/Methanol (0.1% NH₄OH) Gradient: Isocratic 60% B |
| 32 33 | N-(Isoxazol-3-yl)-6- methyl-7,8-dihydro- 6H- cyclopenta[e]imidazo [1,2-a]pyridine-4- carboxamide (enantiomer 1) N-(Isoxazol-3-yl)-6- methyl-7,8-dihydro- 6H- cyclopenta[e]imidazo [1,2-a]pyridine-4- carboxamide (enantiomer 2) | 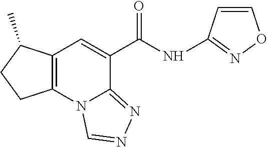 [SMILES: C[C@H]1CCc2c1cc(C(=O)Nc1ccon1)c1nncn21] or 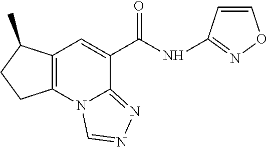 [SMILES: C[C@@H]1CCc2c1cc(C(=O)Nc1ccon1)c1nncn21] | 284.2 284.3 | Column: DAICEL CHIRALCEL OX (250 mm * 30 mm, 10 μm) Mobile phase B: Methanol (0.1% NH₄OH) Gradient: Isocratic 55% B |
| 34 35 | 6-Methyl-N-(5- (trifluoromethyl)95yridine- 2-yl)-7,8-dihydro- 6H- cyclopenta[e][1,2,4] triazolo[4,3-a]pyridine-4- carboxamide (enantiomer 2) 6-Methyl-N-(5- (trifluoromethyl)pyridin- 2-yl)-7,8-dihydro- 6H- cyclopenta[e][1,2,4] triazolo[4,3-a]pyridine-4- carboxamide (enantiomer 1) | 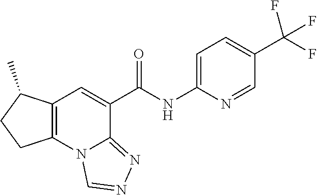 [SMILES: C[C@H]1CCc2c1cc(C(=O)Nc1ccc(C(F)(F)F)cn1)c1nncn21] or 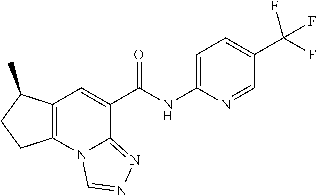 [SMILES: C[C@@H]1CCc2c1cc(C(=O)Nc1ccc(C(F)(F)F)cn1)c1nncn21] | 362.1 362.3 | Column: DAICEL CHIRALCEL OX (250 mm * 30 mm, 10 μm) Mobile phase B: Methanol (0.1% NH₄OH) Gradient: Isocratic 45% B |
| 36 37 | N-(3-Fluoropyridin-2- yl)-6-methyl-7,8- dihydro-6H- cyclopenta[e][1,2,4] triazolo[4,3-a]pyridine-4- carboxamide (enantiomer 2) N-(3-Fluoropyridin-2- yl)-6-methyl-7,8- dihydro-6H- cyclopenta[e][1,2,4] triazolo[4,3-a]pyridine-4- carboxamide (enantiomer 1) | 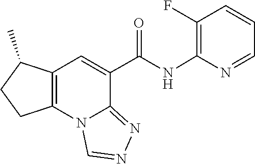 [SMILES: C[C@H]1CCc2c1cc(C(=O)Nc1ncccc1F)c1nncn21] or 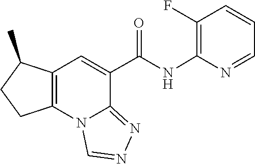 [SMILES: C[C@@H]1CCc2c1cc(C(=O)Nc1ncccc1F)c1nncn21] | 312.5 312.5 | Column: DAICEL CHIRALCEL OX (250 mm * 30 mm, 10 μm) Mobile phase B: 1:1 ACN/Methanol (0.1% NH₄OH) Gradient: Isocratic 60% B |
| 38 39 | N-(Isothiazol-5-yl)-6- methyl-7,8-dihydro- 6H- cyclopenta[e][1,2,4] triazolo[4,3-a]pyridine-4- carboxamide (enantiomer 1) N-(Isothiazol-5-yl)-6- methyl-7,8-dihydro- 6H- cyclopenta[e][1,2,4] triazolo[4,3-a]pyridine-4- carboxamide (enantiomer 2) | 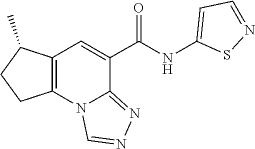 [SMILES: C[C@H]1CCc2c1cc(C(=O)Nc1ccns1)c1nncn21] or 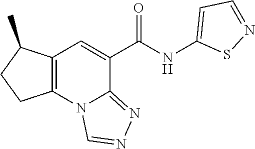 [SMILES: C[C@@H]1CCc2c1cc(C(=O)Nc1ccns1)c1nncn21] | 300.1 300.0 | Column: DAICEL CHIRALCEL OX (250 mm * 30 mm, 10 μm) Mobile phase B: Methanol (0.1% NH₄OH) Gradient: Isocratic 60% B |
| 40 41 | N-(Isothiazol-3-yl)-6- methyl-7,8-dihydro- 6H- cyclopenta[e][1,2,4] triazolo[4,3-a]pyridine-4- carboxamide (enantiomer 1) N-(Isothiazol-3-yl)-6- methyl-7,8-dihydro- 6H- cyclopenta[e][1,2,4] triazolo[4,3-a]pyridine-4- carboxamide (enantiomer 2) | 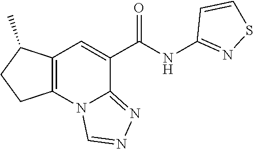 [SMILES: C[C@H]1CCc2c1cc(C(=O)Nc1ccsn1)c1nncn21] or 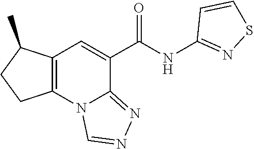 [SMILES: C[C@@H]1CCc2c1cc(C(=O)Nc1ccsn1)c1nncn21] | 300.3 300.2 | Column: DAICEL CHIRALCEL OX (250 mm *3 0 mm, 10 μm) Mobile phase B: 1:1 ACN/Methanol (0.1% NH₄OH) Gradient: Isocratic 60% B |
| 42 43 | N-(5-Fluoropyridin-2- yl)-6-methyl-7,8- dihydro-6H- cyclopenta[e][1,2,4] triazolo[4,3-a]pyridine-4- carboxamide (enantiomer 1) N-(5-Fluoropyridin-2- yl)-6-methyl-7,8- dihydro-6H- cyclopenta[e][1,2,4 ]triazolo[4,3-a]pyridine-4- carboxamide (enantiomer 2) | 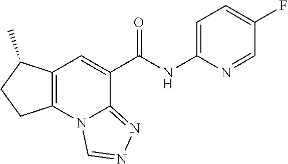 [SMILES: C[C@H]1CCc2c1cc(C(=O)Nc1ccc(F)cn1)c1nncn21] or 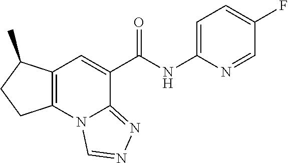 [SMILES: C[C@@H]1CCc2c1cc(C(=O)Nc1ccc(F)cn1)c1nncn21] | 312.2 312.1 | Column: DAICEL CHIRALCEL OX (250 mm * 30 mm, 10 μm) Mobile phase B: Methanol (0.1% NH₄OH) Gradient: Isocratic 50% B |
| 44 45 | N-(Isoxazol-5-yl)-6- methyl-7,8-dihydro- 6H- cyclopenta[e][1,2,4] triazolo[4,3-a]pyridine-4- carboxamide (enantiomer 2) N-(Isoxazol-5-yl)-6- methyl-7,8-dihydro- 6H- cyclopenta[e][1,2,4] triazolo[4,3-a]pyridine-4- carboxamide (enantiomer 1) | 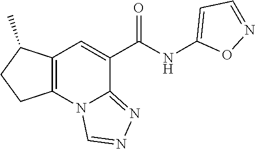 [SMILES: C[C@H]1CCc2c1cc(C(=O)Nc1ccno1)c1nncn21] or 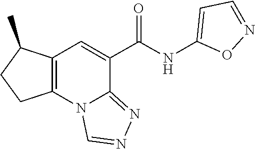 [SMILES: C[C@@H]1CCc2c1cc(C(=O)Nc1ccno1)c1nncn21] | 284.1 284.0 | Column: DAICEL CHIRALCEL OX (250 mm * 30 mm, 10 μm) Mobile phase B: Methanol (0.1% NH₄OH) Gradient: Isocratic 45% B |
| 46 47 | N-(5-Chloropyrimidin- 2-yl)-6-methyl-7,8- dihydro-6H- cyclopenta[e][1,2,4] triazolo[4,3-a]pyridine-4- carboxamide (enantiomer 2) N-(5-Chloropyrimidin- 2-yl)-6-methyl-7,8- dihydro-6H- cyclopenta[e][1,2,4] triazolo[4,3-a]pyridine-4- carboxamide (enantiomer 1) | 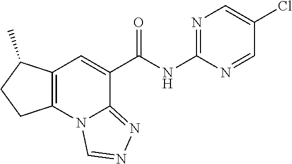 [SMILES: C[C@H]1CCc2c1cc(C(=O)Nc1ncc(Cl)cn1)c1nncn21] or 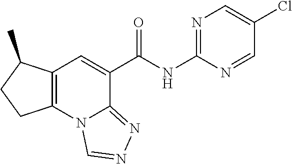 [SMILES: C[C@@H]1CCc2c1cc(C(=O)Nc1ncc(Cl)cn1)c1nncn21] | 328.9 328.9 | Column: DAICEL CHIRALCEL OX (250 mm * 30 mm, 10 μm) Mobile phase B: 1:1 ACN/Methanol (0.1% NH₄OH) Gradient: Isocratic 60% B |
| 48 49 | N-(5- Methoxypyrimidin-2- yl)-6-methyl-7,8- dihydro-6H- cyclopenta[e][1,2,4] triazolo[4,3-a]pyridine-4- carboxamide (enantiomer 1) N-(5- Methoxypyrimidin-2- yl)-6-methyl-7,8- dihydro-6H- cyclopenta[e][1,2,4] triazolo[4,3-a]pyridine-4- carboxamide (enantiomer 2) | 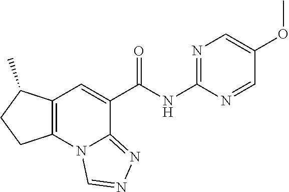 [SMILES: COc1cnc(NC(=O)c2cc3c(n4cnnc24)CC[C@@H]3C)nc1] or 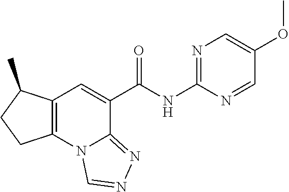 [SMILES: COc1cnc(NC(=O)c2cc3c(n4cnnc24)CC[C@H]3C)nc1] | 325.1 325.1 | Column: DAICEL CHIRALCEL OX (250 mm * 30 mm, 10 μm) Mobile phase B: 1:1 ACN/Methanol (0.1% NH₄OH) Gradient: Isocratic 60% B |
| 50 51 | N-(5-Fluoropyrimidin- 2-yl)-6-isopropoxy-7,8- dihydro-6H- cyclopenta[e][1,2,4] triazolo[4,3-a]pyridine-4- carboxamide (enantiomer 1) N-(5-Fluoropyrimidin- 2-yl)-6-isopropoxy-7,8- dihydro-6H- cyclopenta[e][1,2,4] triazolo[4,3-a]pyridine-4- carboxamide (enantiomer 2) | 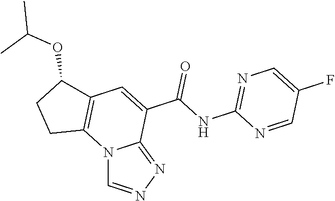 [SMILES: CC(C)O[C@H]1CCc2c1cc(C(=O)Nc1ncc(F)cn1)c1nncn21] or 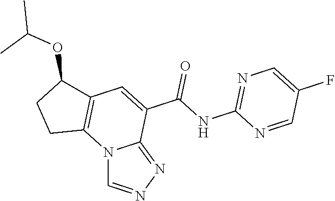 [SMILES: CC(C)O[C@@H]1CCc2c1cc(C(=O)Nc1ncc(F)cn1)c1nncn21] | 357.2 357.2 | Column: DAICEL CHIRALCEL OX (250 mm * 30 mm, 10 μm) Mobile phase B: 1:1 Isopropanol/Methanol (0.1% NH₄OH) Gradient: Isocratic 60% B |
| 52 53 | N-(5-Fluoropyrimidin- 2-yl)-1,6-dimethyl-7,8- dihydro-6H- cyclopenta[e][1,2,4] triazolo[4,3-a]pyridine-4- carboxamide (enantiomer 1) N-(5-Fluoropyrimidin- 2-yl)-1,6-dimethyl-7,8- dihydro-6H- cyclopenta[e][1,2,4] triazolo[4,3-a]pyridine-4- carboxamide (enantiomer 2) | 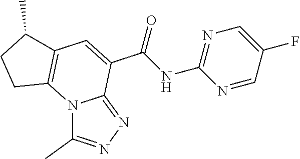 [SMILES: Cc1nnc2c(C(=O)Nc3ncc(F)cn3)cc3c(n12)CC[C@@H]3C] or 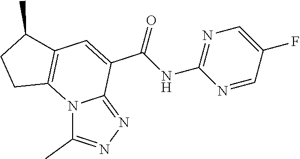 [SMILES: Cc1nnc2c(C(=O)Nc3ncc(F)cn3)cc3c(n12)CC[C@H]3C] | 327.4 327.4 | Mobile phase B: Isopropanol (0.1% NH₄OH) Gradient: Isocratic 35% B Flow Rate: 60 mL/min |
| 54 55 | N-(5-Fluoropyrimidin- 2-yl)-1-isopropyl-6- methyl-7,8-dihydro- 6H- cyclopenta[e][1,2,4] triazolo[4,3-a]pyridine-4- carboxamide (enantiomer 2) N-(5-Fluoropyrimidin- 2-yl)-1-isopropyl-6- methyl-7,8-dihydro- 6H- cyclopenta[e][1,2,4] triazolo[4,3-a]pyridine-4- carboxamide (enantiomer 1) | 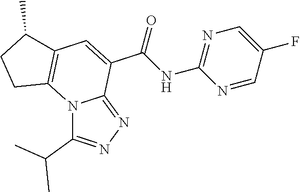 [SMILES: CC(C)c1nnc2c(C(=O)Nc3ncc(F)cn3)cc3c(n12)CC[C@@H]3C] or 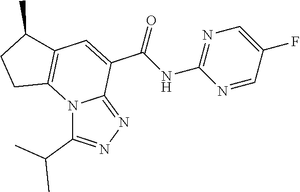 [SMILES: CC(C)c1nnc2c(C(=O)Nc3ncc(F)cn3)cc3c(n12)CC[C@H]3C] | 355.5 355.3 | Column: DAICEL CHIRALCEL IBN (250 mm * 30 mm, 10 μm) Mobile phase B: Ethanol (0.1% NH₄OH) Gradient: Isocratic 35% B |
| 56 57 | 6-Ethyl-N-(5- fluoropyrimidin-2-yl)- 7,8-dihydro-6H- cyclopenta[e][1,2,4] triazolo[4,3-a]pyridine-4- carboxamide (enantiomer 1) 6-Ethyl-N-(5- fluoropyrimidin-2-yl)- 7,8-dihydro-6H- cyclopenta[e][1,2,4] triazolo[4,3-a]pyridine-4- carboxamide (enantiomer 2) | 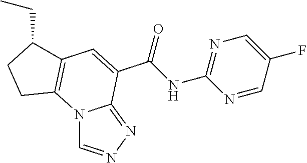 [SMILES: CC[C@H]1CCc2c1cc(C(=O)Nc1ncc(F)cn1)c1nncn21] or 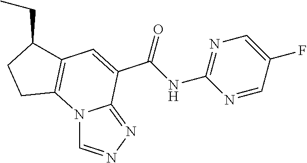 [SMILES: CC[C@@H]1CCc2c1cc(C(=O)Nc1ncc(F)cn1)c1nncn21] | 327.1 327.0 | Column: DAICEL CHIRALCEL AY-H (250 mm * 30 mm, 10 μm) Mobile phase B: Ethanol (0.1% NH₄OH) Gradient: Isocratic 45% B |

The compounds 58 to 68 of Table 3 below were synthesized according to the Schemes 1-3 described above with reference to conditions described for Examples 3 and 4 and separated on a preparatory column under conditions analogous to Separation Condition A (with necessary variation in the mobile phase and gradient as specified in Table 3).

TABLE 3
Examples 58-68

| | | | | Variation |
|---|---|---|---|---|
| | | | ES/MS | from |
| Ex. | | | (m/z) | Separation |
| No. | Chemical Name | Structure | (M + H) | Condition A |
| 58 59 | N-(5-Fluoropyrimidin- 2-yl)-6-isopropyl-7,8- dihydro-6H- cyclopenta[e][1,2,4] triazolo[4,3-a]pyridine-4- carboxamide (enantiomer 2) N-(5-Fluoropyrimidin- 2-yl)-6-isopropyl-7,8- dihydro-6H- cyclopenta[e][1,2,4] triazolo[4,3-a]pyridine-4- carboxamide (enantiomer 1) | 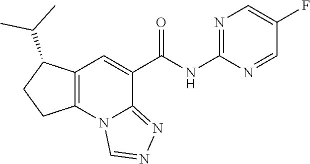 [SMILES: CC(C)[C@H]1CCc2c1cc(C(=O)Nc1ncc(F)cn1)c1nncn21] or 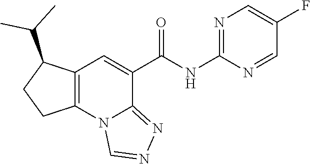 [SMILES: CC(C)[C@@H]1CCc2c1cc(C(=O)Nc1ncc(F)cn1)c1nncn21] | 341.0 341.2 | Mobile phase B: Methanol (0.1% NH₄OH) Gradient: Isocratic 40% B |
| 60 | 6-(sec-butyl)-N-(5- Fluoropyrimidin-2-yl)- 7,8-dihydro-6H- cyclopenta[e][1,2,4] triazolo[4,3-a]pyridine-4- carboxamide (enantiomer 2/2) | 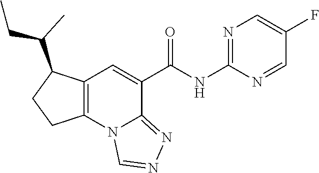 [SMILES: CC[C@@H](C)[C@@H]1CCc2c1cc(C(=O)Nc1ncc(F)cn1)c1nncn21] or 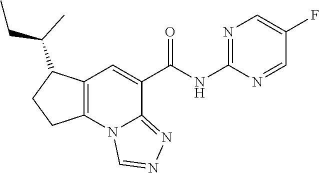 or 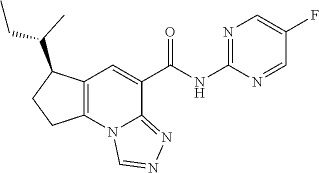 [SMILES: CC[C@H](C)[C@@H]1CCc2c1cc(C(=O)Nc1ncc(F)cn1)c1nncn21] or 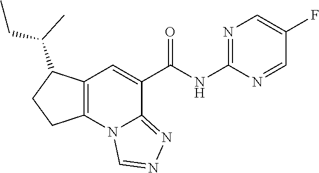 [SMILES: CC[C@H](C)C1CCc2c1cc(C(=O)Nc1ncc(F)cn1)c1nncn21] | 355.2 | Separation 1: Column: DAICEL CHIRALCEL OX 250 × 30 mm I.D., 10 μm Mobile phase B: 1:1 ACN/EtOH (0.1% NH₄OH) Gradient: Isocratic 55% B; Separation 2: Column: DAICEL CHIRALCEL OX 250 × 30 mm I.D., 10 μm Mobile phase B: 1:1 ACN/EtOH (0.1% NH₄OH) Gradient: Isocratic 55% B |
| 61 62 | 6-Methyl-N-(2-methyl- 2H-1,2,3-triazol-4-yl)- 7,8-dihydro-6H- cyclopenta[e][1,2,4] triazolo[4,3-a]pyridine-4- carboxamide (enantiomer 2) 6-Methyl-N-(2-methyl- 2H-1,2,3-triazol-4-yl)- 7,8-dihydro-6H- cyclopenta[e][1,2,4] triazolo[4,3-a]pyridine-4- carboxamide (enantiomer 1) | 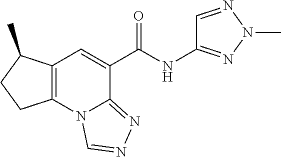 [SMILES: C[C@@H]1CCc2c1cc(C(=O)Nc1cnn(C)n1)c1nncn21] or 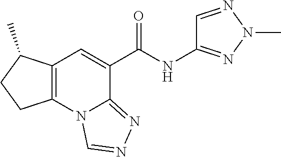 [SMILES: C[C@H]1CCc2c1cc(C(=O)Nc1cnn(C)n1)c1nncn21] | 298.1 298.2 | Column: DAICEL CHIRALCEL OX 250 × 30 mm I.D., 10 μm phase Mobile B: Methanol (0.1% NH₄OH) Gradient: Isocratic 60% B |
| 63 | 6-Methyl-N-(1-methyl- 1H-pyrazol-3-yl)-7,8- dihydro-6H- cyclopenta[e][1,2,4] triazolo[4,3-a]pyridine-4- carboxamide (enantiomer 1) | 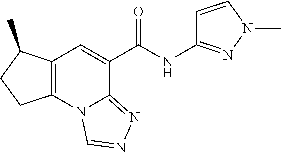 [SMILES: C[C@@H]1CCc2c1cc(C(=O)Nc1ccn(C)n1)c1nncn21] or 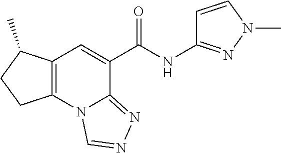 [SMILES: C[C@H]1CCc2c1cc(C(=O)Nc1ccn(C)n1)c1nncn21] | 297.2 | Column: DAICEL CHIRALCEL OX 250 × 30 mm I.D., 10 μm Mobile phase B: 1:1 ACN/MeOH (0.1% NH₄OH) Gradient: Isocratic 60% B |
| 64 65 | 6-Cyclobutyl-N-(5- fluoropyrimidin-2-yl)- 7,8-dihydro-6H- cyclopenta[e][1,2,4] triazolo[4,3-a]pyridine-4- carboxamide (enantiomer 2) 6-Cyclobutyl-N-(5- fluoropyrimidin-2-yl)- 7,8-dihydro-6H- cyclopenta[e][1,2,4] triazolo[4,3-a]pyridine-4- carboxamide (enantiomer 1) | 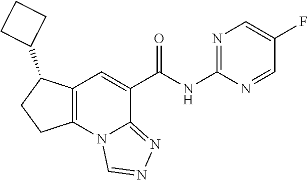 [SMILES: O=C(Nc1ncc(F)cn1)c1cc2c(n3cnnc13)CC[C@@H]2C1CCC1] or 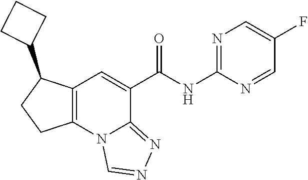 [SMILES: O=C(Nc1ncc(F)cn1)c1cc2c(n3cnnc13)CC[C@H]2C1CCC1] | 353.0 353.0 | Column: DAICEL CHIRALCEL OX 250 × 30 mm I.D., 10 μm Mobile phase B: 1:1 ACN/MeOH (0.1% NH₄OH) Gradient: Isocratic 60% B |
| 66 67 | 6-Cyclopropyl-N-(5- fluoropyrimidin-2-yl)- 7,8-dihydro-6H- cyclopenta[e][1,2,4] triazolo[4,3-a]pyridine-4- carboxamide (enantiomer 2) 6-Cyclopropyl-N-(5- fluoropyrimidin-2-yl)- 7,8-dihydro-6H- cyclopenta[e][1,2,4] triazolo[4,3-a]pyridine-4- carboxamide (enantiomer 1) | 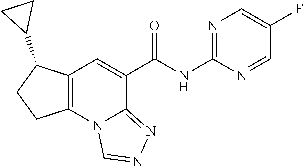 [SMILES: O=C(Nc1ncc(F)cn1)c1cc2c(n3cnnc13)CC[C@@H]2C1CC1] | 339.3 339.1 | Column: DAICEL CHIRALCEL OX 250 × 30 mm I.D., 10 μm Mobile phase B: 1:1 ACN/MeOH (0.1% NH₄OH) Gradient: Isocratic 60% B |
| 68 | N-(5-Fluoropyrimidin- 2-yl)-6-propyl-7,8- dihydro-6H- cyclopenta[e][1,2,4] triazolo[4,3-a]pyridine-4- carboxamide (enantiomer 1) | 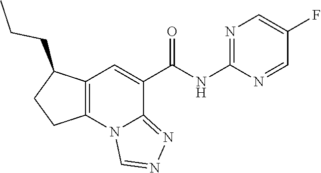 [SMILES: CCC[C@@H]1CCc2c1cc(C(=O)Nc1ncc(F)cn1)c1nncn21] or 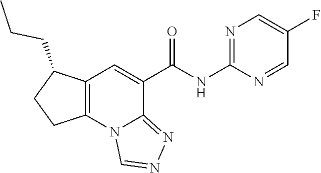 [SMILES: CCC[C@H]1CCc2c1cc(C(=O)Nc1ncc(F)cn1)c1nncn21] | 341.1 | Column: DAICEL CHIRALCEL OX 250 × 30 mm I.D., 10 μm Mobile phase B: 1:1 ACN/MeOH (0.1% NH₄OH) Gradient: Isocratic 60% B |

### EXAMPLE 69

### (S)—N-(5-Fluoropyrimidin-2-yl)-6-methyl-7,8-dihydro-6H cyclopenta[e][1,2,4]triazolo[4,3-a]pyridine-4-carboxamide

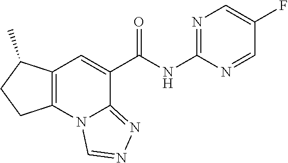 [SMILES: C[C@H]1CCc2c1cc(C(=O)Nc1ncc(F)cn1)c1nncn21]

### Step 1

### Methyl (5S)-2-chloro-5-methyl-6,7-dihydro-5H-cyclopenta[b]pyridine-3-carboxylate

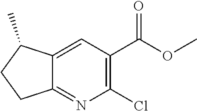

Phosphoryl chloride (POCl₃) (740 g) was added to a solution of methyl (5S)-5-methyl-2-oxo-1,5,6,7-tetrahydrocyclopenta[b]pyridine-3-carboxylate (100 g) and DMF (705 mg). The mixture was stirred at 100° C. for 12 hrs. The reaction mixture was concentrated under reduced pressure to remove POCl₃. The residue was diluted with water (4.00 L, 5 V) and extracted with EtOAc (1.60 L×3). The combined organic layers were washed with saturated aqueous NaCl (800 mL, 1 V), dried over Na₂SO₄, filtered, and concentrated under reduced pressure to give methyl (5S)-2-chloro-5-methyl-6,7-dihydro-5H-cyclopenta[b]pyridine-3-carboxylate (985 g) as a brown solid.

### Step 2

### (5S)-2-Chloro-5-methyl-6,7-dihydro-5H-cyclopenta[b]pyridine-3-carboxylic acid

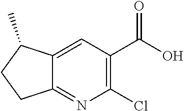

Sodium hydroxide NaOH (232 g) was added to a solution of methyl (5S)-2-chloro-5-methyl-6,7-dihydro-5H-cyclopenta[b]pyridine-3-carboxylate (328 g) in H₂O (984 mL) and THE (984 mL). Methanol (984 mL, 3 V) was added at 25-30° C. The mixture was stirred at 25° C. for 0.5 hr. The reaction mixture was concentrated under reduced pressure to remove solvent. The pH of mixture was adjusted to approximately 2-3 with 3 M HCl (10 V) at 5-10° C. Solid was gradually separated out of the mixture and filtered to give (5S)-2-chloro-5-methyl-6,7-dihydro-5H-cyclopenta[b]pyridine-3-carboxylic acid (550 g, 67.5%) as a white solid.

### Step 3

### (5S)-2-Hydrazino-5-methyl-6,7-dihydro-5H-cyclopenta[b]pyridine-3-carboxylic acid

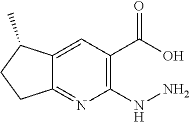

N₂H₄.H₂O (3.32 kg) was added to a solution of (5S)-2-chloro-5-methyl-6,7-dihydro-5H-cyclopenta[b]pyridine-3-carboxylic acid (275 g) in dioxane (1.65 L, 6 V). The mixture was stirred at 80° C. for 16 hrs. The reaction mixture was concentrated under reduced pressure to remove N₂H₄.H₂O. The residue was diluted with MeCN (2.20 L, 4 V) then filtered and removed the insoluble. The combined organic layers were concentrated under reduced pressure to give (5S)-2-hydrazino-5-methyl-6,7-dihydro-5H-cyclopenta[b]pyridine-3-carboxylic acid (1.30 kg, crude) as a brown solid.

### Step 4

### (S)-6-Methyl-7,8-dihydro-6H-cyclopenta[e][1,2,4]triazolo[4,3-a]pyridine-4-carboxylic acid

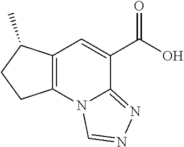 [SMILES: C[C@H]1CCc2c1cc(C(=O)O)c1nncn21]

A solution of (5S)-2-hydrazino-5-methyl-6,7-dihydro-5H-cyclopenta[b]pyridine-3-carboxylic acid (1.30 kg) in formic acid (5.20 L, 4 V) was stirred at 100° C. for 12 hrs. The reaction mixture was concentrated under reduced pressure to remove solvent. The residue was diluted with ice water (3.25 L, 2.5 V) and stirred at 0° C. for 1 hr. The mixture was filtered and washed with water (3.90 L, 3 V), and filtered to give (S)-6-methyl-7,8-dihydro-6H-cyclopenta[e][1,2,4]triazolo[4,3-a]pyridine-4-carboxylic acid (360 g, 64%) as a brown solid.

### Step 5

### (S)—N-(5-Fluoropyrimidin-2-yl)-6-methyl-7,8-dihydro-6H cyclopenta[e][1,2,4]triazolo[4,3-a]pyridine-4-carboxamide

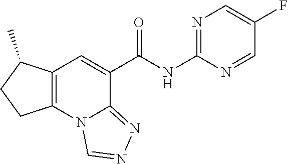 [SMILES: C[C@H]1CCc2c1cc(C(=O)Nc1ncc(F)cn1)c1nncn21]

A mixture of pyridine (1.11 kg) and 5-fluoropyrimidin-2-amine (296 g) was added to a solution of (S)-6-methyl-7,8-dihydro-6H-cyclopenta[e][1,2,4]triazolo[4,3-a]pyridine-4-carboxylic acid (412 g) in DCM (2.47 L, 6 V). POCl₃ (402 g) was added dropwise into the mixture at 25° C. The mixture was stirred at 25° C. for 0.5 hr. The reaction mixture was quenched by addition H₂O (2.00 L, 5 V) at 25° C., and then extracted with DCM (2.46 L, 2 V×3). The combined organic layers were washed with saturated aqueous NaCl (410 mL, 1 V), dried over Na₂SO₄, filtered, and concentrated under reduced pressure to give a residue. The residue was purified by silica gel column chromatography eluting with DCM in EtOAc (I/O to 1/1) to give (S)—N-(5-fluoropyrimidin-2-yl)-6-methyl-7,8-dihydro-6H cyclopenta[e][1,2,4]triazolo[4,3-a]pyridine-4-carboxamide (365 g, 61.7% yield, 99.6 purity) as an off-white solid.

### hAHR Nuclear Translocation Assay

The purpose of this assay is to measure the ability of compounds to bind to, activate, and induce the translocation of AhR into the nucleus of a cell for transcription. Stable cell lines were established using Jump-In™ T-REx™ HEK293 Retargeting Kit (Life Technologies). Human AhR cDNA was cloned into the pJTI R4 CMV-TO EGFP vector. The EGFP was cloned to the C-terminal of AHR to form AhR-EGFP chimera. The pJTI R4 CMV-TO AhR-EGFP vector was transfected using FuGENE® HD into Jump-In™ T-REx™ HEK293 cells. Transfected cells were selected using 2.5 mg/ml G418 for 10 to 14 days, then expanded, harvested, and suspended in freeze media (FBS with 8% DMSO) at 2×10⁷ cells/ml, and aliquots were stored in liquid nitrogen. One day before the assay date, cells were thawed and resuspended in DMEM with 5% FBS in the presence of 1 μg/ml Doxycycline and plated into ploy-L-Lysine coated CELLCARRIER-384 ULTRA Microplates (Perkin Elmer) at 12,000 to 15,000 cells per well and incubated at 37° C. and 5% CO₂ overnight. On the assay date, compound was serially diluted (1:2) into 384-well nunc plates with DMSO using acoustic dispensing (ECHO®). The dose response was a 20-point curve. Compound was resuspended in 40 μl of DMEM plus 0.1% BSA. The culture media was damped and 25 μl of DMEM plus 0.1% BSA was added, then 25 μL of compound in DMEM plus 0.1% BSA was added into cell plates. Cells were incubated compounds at 37° C. and 5% CO₂ for 45 minutes. The final DMSO concentration was 0.2%. The media was damped after 45 minutes incubation. The cells were fixed with 40 μl of cold MeOH (−20° C.) for 20 minutes. The MeOH was damped and 50 μL of DPBS containing 1 μg/mL Hochst was added into the cell plates. The intensity of EGFP was quantitated by using Opera PHENIX® or OPERETTA® high content image system (Perkin Elmer) with 20× Water Objective and five field per well. The ratio of EGFP fluorescent intensity in nuclear over cytosol was analyzed using a 4-parameter nonlinear logistic equation to determine the potency of AhR agonists.

Table 3 shows the hAHIR nuclear translocation assay data for certain compounds of examples above. The designation “A” refers to EC₅₀<=1 nM; “B” refers to 1 nM<EC₅₀<=10 nM; “C” refers to 10 nM<EC₅₀<=50 nM; and “D” refers to EC₅₀>50 nM. “-” indicates activity not tested.

TABLE 3
hAHR Nuclear Translocation Assay EC₅₀ Values

| Example No. | Rel EC₅₀ (nM) |
|---|---|
| 1 | 3.1 |
| 2 | 0.585 |
| 3 | 5.37 |
| 4 | D |
| 5 | 5.79 |
| 6 | 5.74 |
| 7 | 11.1 |
| 8 | 25.3 |
| 9 | 3.37 |
| 10 | 1.32 |
| 11 | 1.58 |
| 12 | 9.8 |
| 13 | 0.495 |
| 14 | 1.66 |
| 15 | — |
| 16 | A |
| 17 | A |
| 18 | A |
| 19 | A |
| 20 | D |
| 21 | D |
| 22 | A |
| 23 | B |
| 24 | B |
| 25 | B |
| 26 | B |
| 27 | B |
| 28 | C |
| 29 | C |
| 30 | C |
| 31 | C |
| 32 | B |
| 33 | D |
| 34 | B |
| 35 | B |
| 36 | B |
| 37 | B |
| 38 | B |
| 39 | B |
| 40 | A |
| 41 | B |
| 42 | B |
| 43 | B |
| 44 | D |
| 45 | C |
| 46 | B |
| 47 | B |
| 48 | C |
| 49 | C |
| 50 | D |
| 51 | D |
| 52 | D |
| 53 | D |
| 54 | D |
| 55 | D |
| 56 | B |
| 57 | B |
| 58 | B |
| 59 | C |
| 60 | C |
| 61 | B |
| 62 | C |
| 63 | C |
| 64 | B |
| 65 | B |
| 66 | B |
| 67 | B |
| 68 | C |

Certain compounds of the present disclosure are novel agonist of the aryl hydrocarbon receptor (AHR), as demonstrate by hAHR Nuclear Translocation Assay illustrated above. Additional compounds of Formula I-1, I-2, I-3, II-1, II-2, II-3, II-1A, III-1, III-2, III-3, III-1A, IV-1, IV-2, IV-3, IV-1A, V-1, V-2, V-3, or V-4, can be shown to be AHR agonists using the same or similar assay techniques described above. These compounds and the examples provided herein, are believed to be useful in the treatment of immune-mediated diseases (IMD), in particular psoriasis and atopic dermatitis, among others.

What is claimed is:

1. A compound of formula: 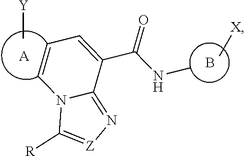
wherein:
ring A is a 5-membered or 6-membered carbocycle;
ring B is a phenyl, or a 5-membered or 6-membered heteroaryl having 1 or 3 heteroatoms, wherein each heteroatom of the heteroaryl is independently selected from N, S, and O;
R is H or C₁₋₃ alkyl;
X is H, halo, C₁₋₃ alkyl optionally substituted with one or more halo, or a C₁₋₃ alkoxyl;
Y is H, C₁₋₄ alkyl, or C₃₋₄ cycloalkyl, wherein the C₁₋₄ alkyl is a primary or secondary alkyl; and
Z is CH or N,
a stereoisomer or mixture of stereoisomers thereof, or a pharmaceutically acceptable salt of each thereof.

2. The compound of claim 1, wherein R is H, a stereoisomer or mixture of stereoisomers thereof, or a pharmaceutically acceptable salt of each thereof.

3. The compound of claim 1, wherein 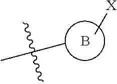
is selected from 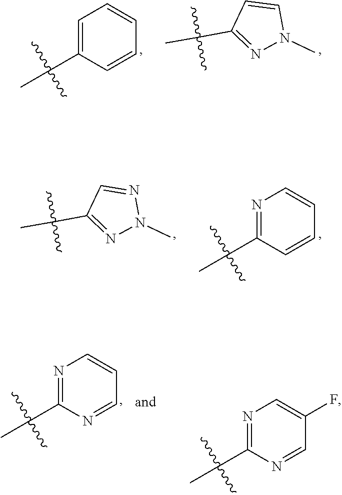
a stereoisomer or mixture of stereoisomers thereof, or a pharmaceutically acceptable salt of each thereof.

4. The compound of claim 1, having formula: 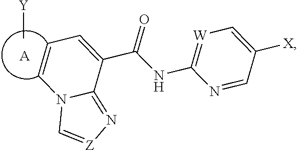
wherein:
X is H or halo;
Y is H or C₁₋₃ alkyl; and
W is CH or N,
a stereoisomer or mixture of stereoisomers thereof, or a pharmaceutically acceptable salt of each thereof.

5. The compound of claim 1, wherein 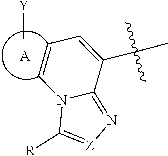
is selected from the group consisting of: 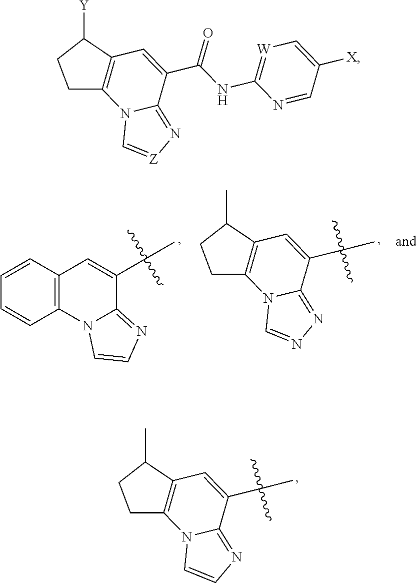
a stereoisomer or mixture of stereoisomers thereof, or a pharmaceutically acceptable salt of each thereof.

6. The compound of claim 1, wherein ring A is phenyl, or a pharmaceutically acceptable salt of each thereof.

7. The compound of claim 1, wherein ring A is 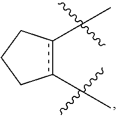
a stereoisomer or mixture of stereoisomers thereof, or a pharmaceutically acceptable salt of each thereof.

8. The compound of claim 1, having the formula: 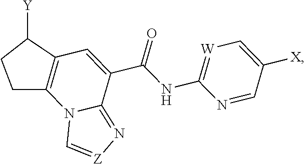
wherein:
X is H or halo;
Y is H or C₁₋₃ alkyl; and
W is CH or N,
a stereoisomer or mixture of stereoisomers thereof, or a pharmaceutically acceptable salt of each thereof.

9. The compound of claim 1, wherein Z is N, a stereoisomer or mixture of stereoisomers thereof, or a pharmaceutically acceptable salt of each thereof.

10. The compound of claim 1, wherein Z is CH, a stereoisomer or mixture of stereoisomers thereof, or a pharmaceutically acceptable salt of each thereof.

11. The compound of claim 4, wherein W is N, a stereoisomer or mixture of stereoisomers thereof, or a pharmaceutically acceptable salt of each thereof.

12. The compound of claim 1, wherein X is F, a stereoisomer or mixture of stereoisomers thereof, or a pharmaceutically acceptable salt of each thereof.

13. The compound of claim 1, wherein Y is methyl, a stereoisomer or mixture of stereoisomers thereof, or a pharmaceutically acceptable salt of each thereof.

14. The compound of claim 1, selected from the table below, a stereoisomer or mixture of stereoisomers thereof, or a pharmaceutically acceptable salt of each thereof:

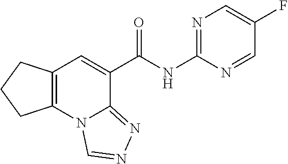 [SMILES: O=C(Nc1ncc(F)cn1)c1cc2c(n3cnnc13)CCC2]

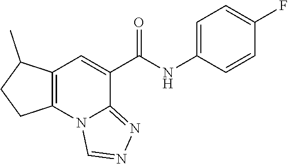 [SMILES: CC1CCc2c1cc(C(=O)Nc1ccc(F)cc1)c1nncn21]

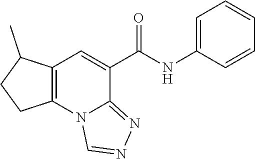 [SMILES: CC1CCc2c1cc(C(=O)Nc1ccccc1)c1nncn21]

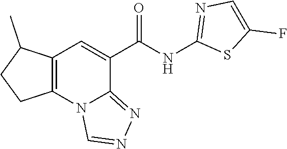 [SMILES: CC1CCc2c1cc(C(=O)Nc1ncc(F)s1)c1nncn21]

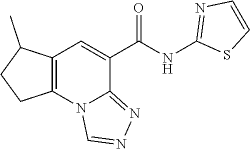 [SMILES: CC1CCc2c1cc(C(=O)Nc1nccs1)c1nncn21]

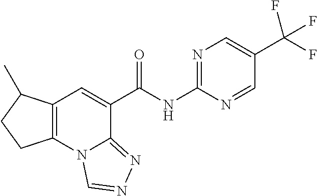 [SMILES: CC1CCc2c1cc(C(=O)Nc1ncc(C(F)(F)F)cn1)c1nncn21]

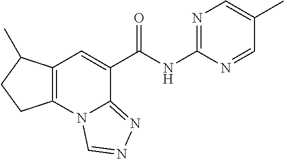 [SMILES: Cc1cnc(NC(=O)c2cc3c(n4cnnc24)CCC3C)nc1]

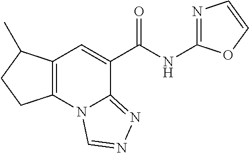 [SMILES: CC1CCc2c1cc(C(=O)Nc1ncco1)c1nncn21]

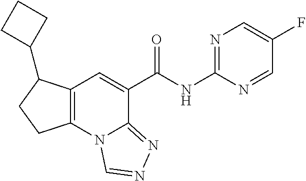 [SMILES: O=C(Nc1ncc(F)cn1)c1cc2c(n3cnnc13)CCC2C1CCC1]

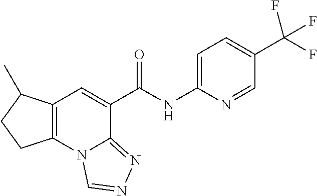 [SMILES: CC1CCc2c1cc(C(=O)Nc1ccc(C(F)(F)F)cn1)c1nncn21]

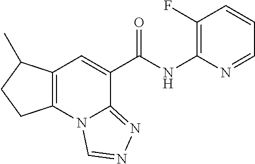 [SMILES: CC1CCc2c1cc(C(=O)Nc1ncccc1F)c1nncn21]

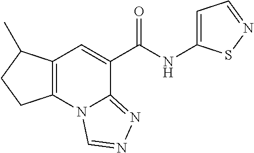 [SMILES: CC1CCc2c1cc(C(=O)Nc1ccns1)c1nncn21]

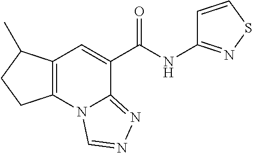 [SMILES: CC1CCc2c1cc(C(=O)Nc1ccsn1)c1nncn21]

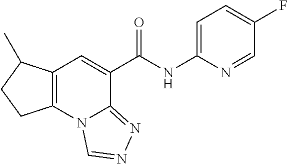 [SMILES: CC1CCc2c1cc(C(=O)Nc1ccc(F)cn1)c1nncn21]

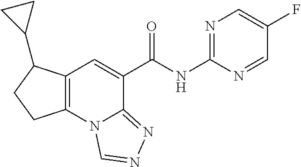 [SMILES: O=C(Nc1ncc(F)cn1)c1cc2c(n3cnnc13)CCC2C1CC1]

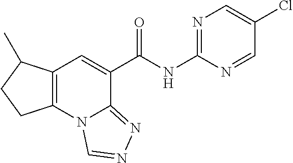 [SMILES: CC1CCc2c1cc(C(=O)Nc1ncc(Cl)cn1)c1nncn21]

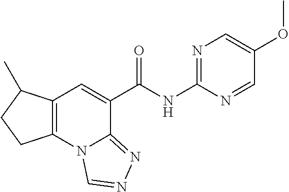 [SMILES: COc1cnc(NC(=O)c2cc3c(n4cnnc24)CCC3C)nc1]

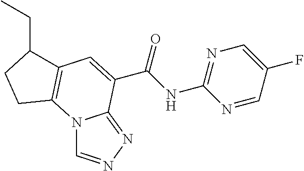 [SMILES: CCC1CCc2c1cc(C(=O)Nc1ncc(F)cn1)c1nncn21]

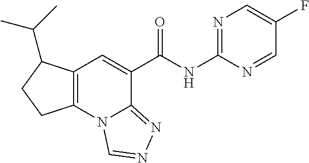 [SMILES: CC(C)C1CCc2c1cc(C(=O)Nc1ncc(F)cn1)c1nncn21]

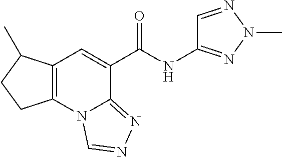

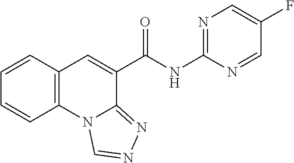 [SMILES: O=C(Nc1ncc(F)cn1)c1cc2ccccc2n2cnnc12]

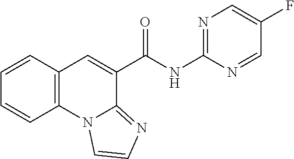 [SMILES: O=C(Nc1ncc(F)cn1)c1cc2ccccc2n2ccnc12]

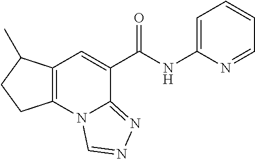 [SMILES: CC1CCc2c1cc(C(=O)Nc1ccccn1)c1nncn21]

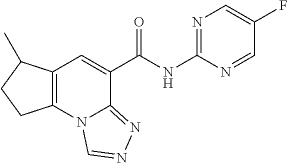 [SMILES: CC1CCc2c1cc(C(=O)Nc1ncc(F)cn1)c1nncn21]

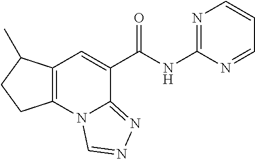 [SMILES: CC1CCc2c1cc(C(=O)Nc1ncccn1)c1nncn21]

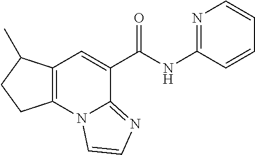 [SMILES: CC1CCc2c1cc(C(=O)Nc1ccccn1)c1nccn21]

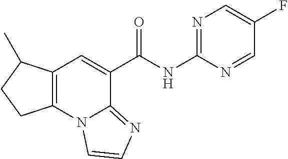 [SMILES: CC1CCc2c1cc(C(=O)Nc1ncc(F)cn1)c1nccn21]

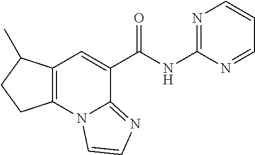 [SMILES: CC1CCc2c1cc(C(=O)Nc1ncccn1)c1nccn21]

15. The compound of claim 1, selected from the following table, or a pharmaceutically acceptable salt thereof:

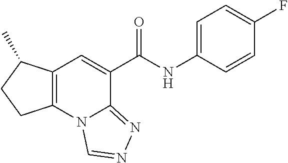 [SMILES: C[C@H]1CCc2c1cc(C(=O)Nc1ccc(F)cc1)c1nncn21]

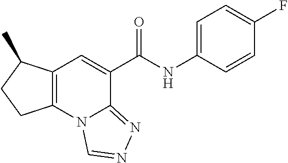 [SMILES: C[C@@H]1CCc2c1cc(C(=O)Nc1ccc(F)cc1)c1nncn21]

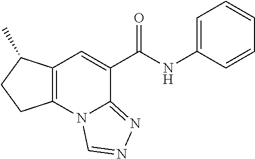 [SMILES: C[C@H]1CCc2c1cc(C(=O)Nc1ccccc1)c1nncn21]

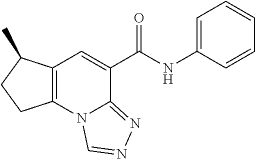 [SMILES: C[C@@H]1CCc2c1cc(C(=O)Nc1ccccc1)c1nncn21]

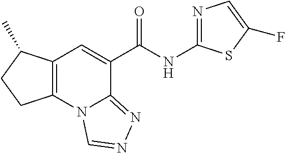 [SMILES: C[C@H]1CCc2c1cc(C(=O)Nc1ncc(F)s1)c1nncn21]

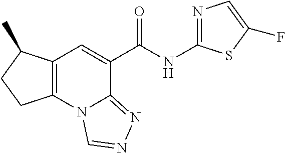 [SMILES: C[C@@H]1CCc2c1cc(C(=O)Nc1ncc(F)s1)c1nncn21]

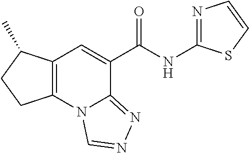 [SMILES: C[C@H]1CCc2c1cc(C(=O)Nc1nccs1)c1nncn21]

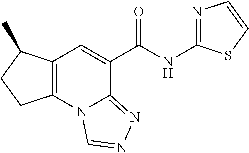 [SMILES: C[C@@H]1CCc2c1cc(C(=O)Nc1nccs1)c1nncn21]

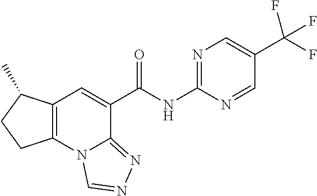 [SMILES: C[C@H]1CCc2c1cc(C(=O)Nc1ncc(C(F)(F)F)cn1)c1nncn21]

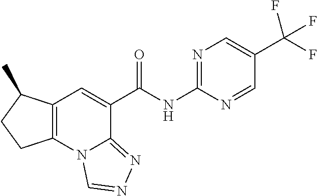 [SMILES: C[C@@H]1CCc2c1cc(C(=O)Nc1ncc(C(F)(F)F)cn1)c1nncn21]

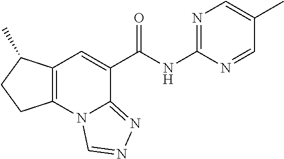 [SMILES: Cc1cnc(NC(=O)c2cc3c(n4cnnc24)CC[C@@H]3C)nc1]

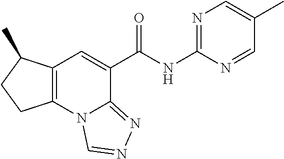 [SMILES: Cc1cnc(NC(=O)c2cc3c(n4cnnc24)CC[C@H]3C)nc1]

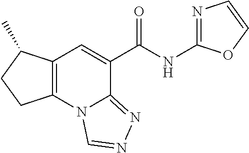 [SMILES: C[C@H]1CCc2c1cc(C(=O)Nc1ncco1)c1nncn21]

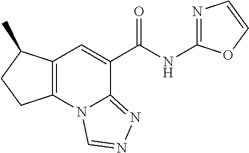 [SMILES: C[C@@H]1CCc2c1cc(C(=O)Nc1ncco1)c1nncn21]

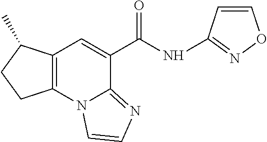 [SMILES: C[C@H]1CCc2c1cc(C(=O)Nc1ccon1)c1nccn21]

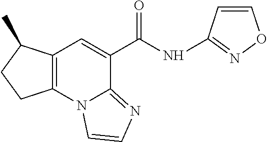 [SMILES: C[C@@H]1CCc2c1cc(C(=O)Nc1ccon1)c1nccn21]

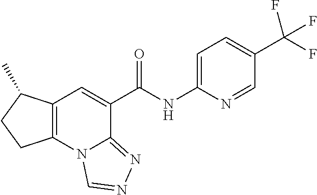 [SMILES: C[C@H]1CCc2c1cc(C(=O)Nc1ccc(C(F)(F)F)cn1)c1nncn21]

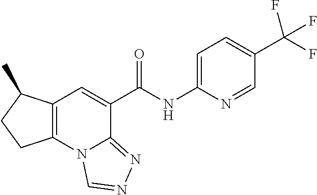 [SMILES: C[C@@H]1CCc2c1cc(C(=O)Nc1ccc(C(F)(F)F)cn1)c1nncn21]

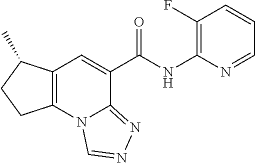 [SMILES: C[C@H]1CCc2c1cc(C(=O)Nc1ncccc1F)c1nncn21]

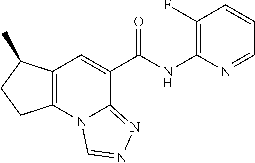 [SMILES: C[C@@H]1CCc2c1cc(C(=O)Nc1ncccc1F)c1nncn21]

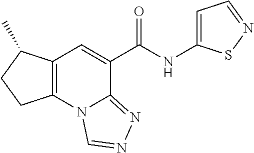 [SMILES: C[C@H]1CCc2c1cc(C(=O)Nc1ccns1)c1nncn21]

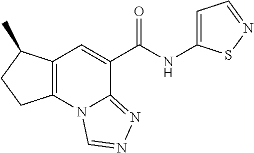 [SMILES: C[C@@H]1CCc2c1cc(C(=O)Nc1ccns1)c1nncn21]

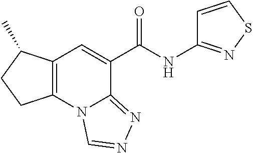 [SMILES: C[C@H]1CCc2c1cc(C(=O)Nc1ccsn1)c1nncn21]

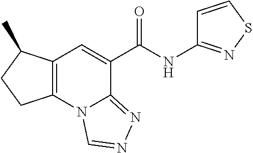 [SMILES: C[C@@H]1CCc2c1cc(C(=O)Nc1ccsn1)c1nncn21]

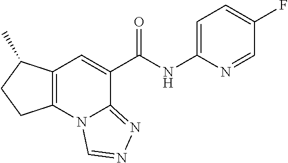 [SMILES: C[C@H]1CCc2c1cc(C(=O)Nc1ccc(F)cn1)c1nncn21]

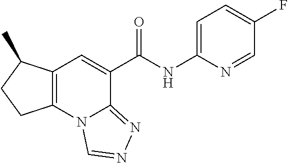 [SMILES: C[C@@H]1CCc2c1cc(C(=O)Nc1ccc(F)cn1)c1nncn21]

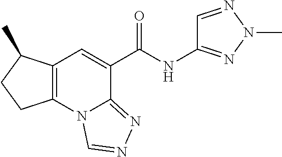 [SMILES: C[C@@H]1CCc2c1cc(C(=O)Nc1cnn(C)n1)c1nncn21]

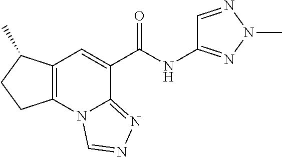 [SMILES: C[C@H]1CCc2c1cc(C(=O)Nc1cnn(C)n1)c1nncn21]

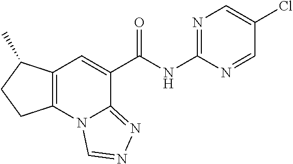 [SMILES: C[C@H]1CCc2c1cc(C(=O)Nc1ncc(Cl)cn1)c1nncn21]

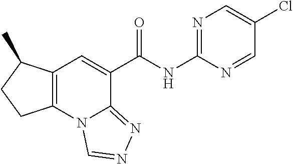 [SMILES: C[C@@H]1CCc2c1cc(C(=O)Nc1ncc(Cl)cn1)c1nncn21]

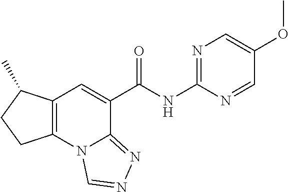 [SMILES: COc1cnc(NC(=O)c2cc3c(n4cnnc24)CC[C@@H]3C)nc1]

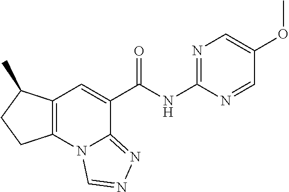 [SMILES: COc1cnc(NC(=O)c2cc3c(n4cnnc24)CC[C@H]3C)nc1]

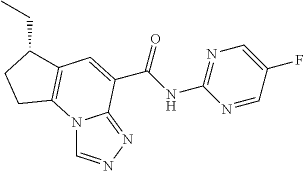 [SMILES: CC[C@H]1CCc2c1cc(C(=O)Nc1ncc(F)cn1)c1nncn21]

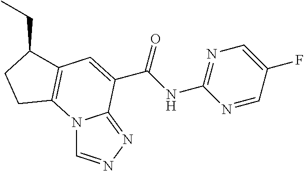 [SMILES: CC[C@@H]1CCc2c1cc(C(=O)Nc1ncc(F)cn1)c1nncn21]

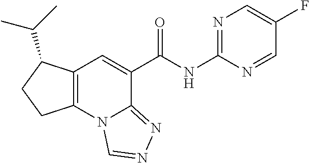 [SMILES: CC(C)[C@H]1CCc2c1cc(C(=O)Nc1ncc(F)cn1)c1nncn21]

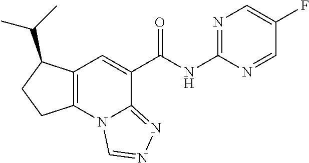 [SMILES: CC(C)[C@@H]1CCc2c1cc(C(=O)Nc1ncc(F)cn1)c1nncn21]

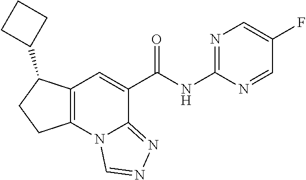 [SMILES: O=C(Nc1ncc(F)cn1)c1cc2c(n3cnnc13)CC[C@@H]2C1CCC1]

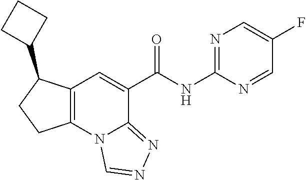 [SMILES: O=C(Nc1ncc(F)cn1)c1cc2c(n3cnnc13)CC[C@H]2C1CCC1]

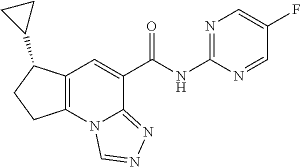 [SMILES: O=C(Nc1ncc(F)cn1)c1cc2c(n3cnnc13)CC[C@@H]2C1CC1]

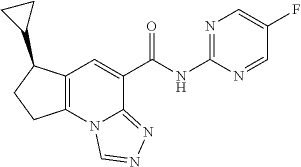 [SMILES: O=C(Nc1ncc(F)cn1)c1cc2c(n3cnnc13)CC[C@H]2C1CC1]

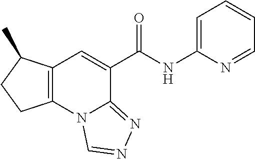 [SMILES: C[C@@H]1CCc2c1cc(C(=O)Nc1ccccn1)c1nncn21]

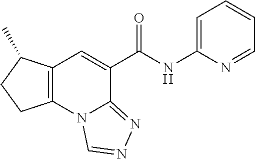 [SMILES: C[C@H]1CCc2c1cc(C(=O)Nc1ccccn1)c1nncn21]

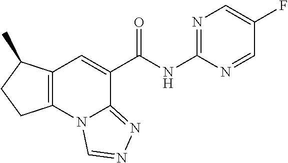 [SMILES: C[C@@H]1CCc2c1cc(C(=O)Nc1ncc(F)cn1)c1nncn21]

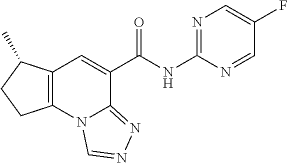 [SMILES: C[C@H]1CCc2c1cc(C(=O)Nc1ncc(F)cn1)c1nncn21]

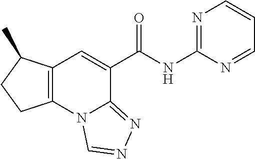 [SMILES: C[C@@H]1CCc2c1cc(C(=O)Nc1ncccn1)c1nncn21]

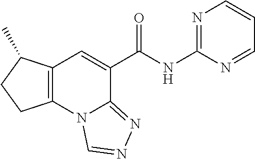 [SMILES: C[C@H]1CCc2c1cc(C(=O)Nc1ncccn1)c1nncn21]

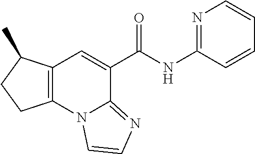 [SMILES: C[C@@H]1CCc2c1cc(C(=O)Nc1ccccn1)c1nccn21]

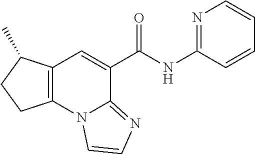 [SMILES: C[C@H]1CCc2c1cc(C(=O)Nc1ccccn1)c1nccn21]

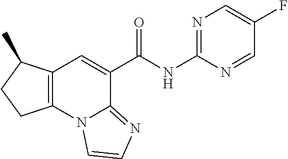 [SMILES: C[C@@H]1CCc2c1cc(C(=O)Nc1ncc(F)cn1)c1nccn21]

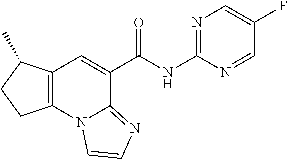 [SMILES: C[C@H]1CCc2c1cc(C(=O)Nc1ncc(F)cn1)c1nccn21]

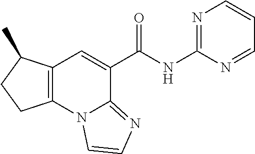 [SMILES: C[C@@H]1CCc2c1cc(C(=O)Nc1ncccn1)c1nccn21]

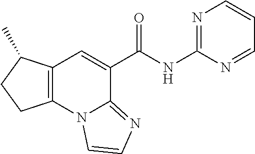 [SMILES: C[C@H]1CCc2c1cc(C(=O)Nc1ncccn1)c1nccn21]

16. The compound of claim 1, having the formula of: 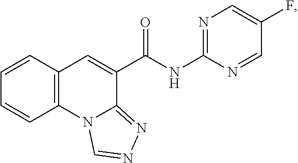 [SMILES: O=C(Nc1ncc(F)cn1)c1cc2ccccc2n2cnnc12]
or a pharmaceutically acceptable salt thereof.

17. The compound of claim 1, having the formula of: 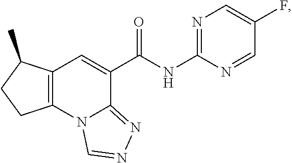 [SMILES: C[C@@H]1CCc2c1cc(C(=O)Nc1ncc(F)cn1)c1nncn21]
a stereoisomer or mixture of stereoisomers thereof, or a pharmaceutically acceptable salt of each thereof.

18. The compound of claim 1, having the formula of: 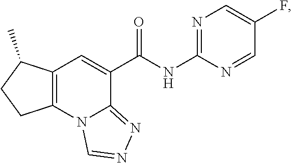 [SMILES: C[C@H]1CCc2c1cc(C(=O)Nc1ncc(F)cn1)c1nncn21]
or a pharmaceutically acceptable salt of each thereof.

19. The compound of claim 1, having the formula of: 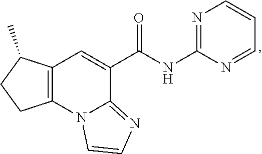 [SMILES: C[C@H]1CCc2c1cc(C(=O)Nc1ncccn1)c1nccn21]
a stereoisomer or mixture of stereoisomers thereof, or a pharmaceutically acceptable salt of each thereof.

20. The compound of claim 1, selected from Table 2, 2A, and 3, a stereoisomer or mixture of stereoisomers thereof, or a pharmaceutically acceptable salt of each thereof.

21. A method of treating a disease or disorder selected from psoriasis, atopic dermatitis, ulcerative colitis, Crohn's disease, graft-versus-host disease, rheumatoid arthritis, and multiple sclerosis in a patient in need thereof, comprising administering to the patient an effective amount of a compound of claim 1, a stereoisomer or mixture of stereoisomers thereof, or a pharmaceutically acceptable salt of each thereof.

22. A compound of the formula: 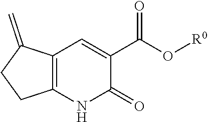
or a salt thereof, wherein R⁰ is C₁₋₃ alkyl.

23. A compound of the formula: 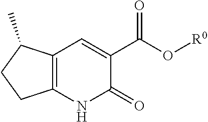
or a salt thereof, wherein R⁰ is C₁₋₃ alkyl.

24. A process for preparing a compound of claim 23, comprising contacting a compound of formula: 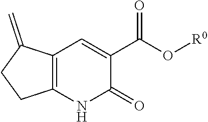
with molecular hydrogen under conditions sufficient to provide the compound of claim 23.
